(19) United States
(12) Patent Application Publication
(10) Pub. No.: US 2026/0176599 A1
(43) Pub. Date: Jun. 25, 2026

(54) VARIANTS OF POLY(A) POLYMERASE AND USES THEREOF

(71) Applicant: DNA Script, Le Kremlin-Bicêtre (FR)

(72) Inventors: Tillmann HEINISCH, Le Kremlin-Bicêtre (FR); Elinor MORRIS, Le Kremlin-Bicêtre (FR); Morgane MELLARE, Le Kremlin-Bicêtre (FR); Guillaume VILLAIN, Le Kremlin-Bicêtre (FR); Clara MENDES, Le Kremlin-Bicêtre (FR); Elise CHAMPION, Le Kremlin-Bicêtre (FR); Mikhael SOSKINE, Le Kremlin-Bicêtre (FR)

(21) Appl. No.: 19/132,344

(22) PCT Filed: Dec. 4, 2023
(86) PCT No.: PCT/EP2023/084053

§ 371 Date: May 22, 2025

(30) Foreign Application Priority Data
Dec. 5, 2022 (EP) 22211517.2
Oct. 13, 2023 (EP) 23306789.1

(51) Int. Cl. C12N 9/12; C12P 19/34

(52) U.S. Cl. CPC C12N 9/1241; C12P 19/34; C12Y 207/07019

(57) ABSTRACT

The present invention relates to poly(A) polymerase variants having several amino acid modifications which confer increased stability, increased activity and increased purity of the polynucleotides obtained therefrom during template-free polynucleotide synthesis. The present invention further relates to use of the variant enzymes for synthesising polynucleotides, methods of synthesising polynucleotides comprising the variant enzymes, and kits comprising the variant enzyme.

### FIELD OF THE INVENTION

The present invention relates to a poly(A) polymerase variant having several amino acid modifications which confer increased stability, increased activity and increased purity of the polynucleotides obtained therefrom, which are also part of the invention. The present invention further relates to uses of the enzyme for synthesising polynucleotides, methods of synthesising polynucleotides comprising the enzyme, and kits comprising the enzyme.

### INTRODUCTION TO THE INVENTION

Interest in enzymatic approaches to polynucleotide synthesis has recently increased both because of increased demand for synthetic polynucleotides that are made to order for use in many areas of biotechnology such as CRISPR-Cas9 applications, high-throughput sequencing, labelling, PCR, and the like, and also due to the limitations of chemical approaches to polynucleotide synthesis, as described in Jensen et al. Biochemistry, 57: 1821-1832 (2018).

Currently, most enzymatic synthesis approaches employ a template-free polymerase to repeatedly added blocked or protected nucleoside triphosphates to the free end of an initiator polynucleotide or subsequently elongated polynucleotide attached to a solid support, followed by rounds of deblocking and polymerisation until the desired polynucleotide is obtained. Typically, such processes employ a terminal deoxynucleotidyl transferase (TdT) because of the benefit of mild non-toxic reaction conditions (WO2015/159023).

However, these TdT enzymes incorporate the modified nucleoside triphosphates required for template-free synthesis processes at low efficiency. Efforts have been made in the field to improve their efficiency by creating modified variants with better incorporation efficiencies, for example (US2019/0211315 or WO2017/216472). Yet, the efficiencies of TdT enzymes still remains below desirable levels.

Alternatively, work has been done to look for different polymerases to use in the template-free synthesis process, such as poly(A) or poly(U) polymerases. These have also been modified to try to improve efficacy (WO2021/018919). However, whilst these modified polymerases may provide better rates of incorporation of modified nucleotides than TdT enzymes, they can have poor stability at higher temperatures and produce polynucleotide products with low purity, especially those polynucleotides which are ‘difficult’ sequences containing a high GC content. In addition, certain modified polymerases can be difficult to produce.

It would be an improvement to current enzymatic polynucleotide synthesis processes if novel template-free polymerases were available with improved stability and/or improved activity and/or improved product purity and/or that can be produced with an increased yield.

It is the aim of one or more aspects of the present invention to solve one or more of the above-mentioned problems in the art.

### SUMMARY OF THE INVENTION

According to a first aspect of the present invention, there is provided a poly(A) polymerase (PAP) variant, or a functional fragment thereof, comprising an amino acid sequence having at least 70% identity to a functional fragment of SEQ ID NO:1, and comprising mutations at residues V199, V240, and M318 of SEQ ID NO:1 or at functionally equivalent residues thereto. According to another aspect of the present invention, there is provided a poly(A) polymerase (PAP) variant, or a functional fragment thereof, comprising an amino acid sequence having at least 70% identity to SEQ ID NO:1, and comprising mutations at residues V199, V240, and M318 of SEQ ID NO:1 or at functionally equivalent residues thereto.

According to another aspect of the present invention, there is provided a poly(A) polymerase (PAP) variant, or a functional fragment thereof, comprising an amino acid sequence having:
at least 70% identity to a functional fragment of SEQ ID NO:1, and
at least 70% identity to a SEQ ID NO:1,

and comprising mutations at residues V199, V240, and M318 of SEQ ID NO:1 or at functionally equivalent residues thereto.

Typically, the PAP variant of the invention has an improved activity and/or improved stability and/or an improved product purity and/or that can be produced with an increased yield compared to the corresponding wild-type PAP.

In one or more embodiments, the PAP variant is capable of synthesizing a polynucleotide. Preferentially, the PAP variant is capable of synthesizing a polynucleotide without a template. Preferentially, the PAP variant is capable of synthesizing a polynucleotide by incorporating a 3′-blocked nucleoside triphosphate and/or a 3′-blocked-2′-deoxynucleoside triphosphate, preferably a 3′-O-azidomethyl-ribonucleoside triphosphate and/or a 3′-O-azidomethyl-2′-deoxyribonucleoside triphosphate, into a nucleic acid fragment to form the polynucleotide. In one or more embodiments, the nucleic acid fragment is a ribonucleic acid fragment or a deoxyribonucleic acid fragment. Preferentially, the PAP variant is capable of incorporating a 3′-O-azidomethyl-ribonucleoside triphosphate into a ribonucleic acid fragment or a 3′-O-azidomethyl-2′-deoxyribonucleoside triphosphate into a deoxyribonucleic acid fragment to form the polynucleotide.

In one or more embodiments, the polynucleotide is a poly-2′-deoxyribonucleotide and the 3′-O-blocked-nucleoside triphosphate is a 3′-O-blocked-2′-deoxyribonucleoside triphosphate. In further embodiments, the 3′-O-blocked-2′-deoxyribonucleotide triphosphate is a 3′-O-azidomethyl-2′-deoxyribonucleoside triphosphate or a 3′-O-amino-2′-deoxyribonucleoside triphosphate.

In further embodiments, the polynucleotide is a polyribonucleotide and said 3′-O-blocked-nucleoside triphosphate is a 3′-O-blocked-ribonucleoside triphosphate. In some embodiments, the 3′-O-blocked-ribonucleoside triphosphate is a 3′-O-azidomethyl-ribonucleoside triphosphate. In further embodiments, the 3′-azidomethyl-O-ribonucleoside triphosphate is selected from the group consisting of 3′-azidomethyl-O-adenosine triphosphate, 3′-azidomethyl-O-guanosine triphosphate, and 3′-azidomethyl-O-cytidine triphosphate, 3′-azidomethyl-O-uridine triphosphate.

In one or more embodiments, the 3′-O-blocked-nucleoside triphosphate can be further modified compared to natural nucleoside triphosphate by replacing the 2′—OH group with various chemical groups such as 2′-O-methyl- or 2′-fluoro-NTPs.

Such modified NTPs or dNTPs can be particularly interesting when producing polynucleotides for certain applications such as CRISPR-Cas genome editing. However, they can be “difficult” to incorporate by polymerases.

In one or more embodiments, the PAP variant further comprises one or more mutations at residues selected from: G71, Y87, A131, F143, N195, H205, P208, M244, P293, A320, Q334, K337, A410, E574, and P643 of SEQ ID NO:1, or at functionally equivalent residues thereto.

In one or more embodiments, the PAP variant further comprises one or more mutations at residues selected from: G71, Y87, F143, N195, H205, P208, M244, P293, A320, A410, E574, and P643 of SEQ ID NO:1, or at functionally equivalent residues thereto.

In one or more embodiments, the PAP variant further comprises one or more mutations at residues selected from: R41, G71, Y87, A131, F143, N195, H205, P208, M244, V289, P293, A320, Q334, K337, E341, K381, S387, I401, A410, K415, M571, E574, E577 and P643 of SEQ ID NO:1, or at functionally equivalent residues thereto.

In one or more embodiments, the PAP variant further comprises one or more mutations at residues selected from: R41, G71, Y87, A131, F143, N195, H205, P208, M244, V289, P293, A320, E341, K381, S387, I401, A410, K415, M571, E574, E577 and P643 of SEQ ID NO:1, or at functionally equivalent residues thereto.

In one or more embodiments, the PAP variant further comprises one or more mutations at residues wherein the enzyme further comprises a mutation at residues selected from R41, V289, E341, K381, S387, I401, K415, M571 and E577 of SEQ ID NO:1: In one or more embodiments, the PAP variant further comprises one or more mutations at residues selected from: G71, Y87, A131, F143, N195, H205, P208, M244, P293, A320, Q334, and K337 of SEQ ID NO:1, or at functionally equivalent residues thereto.

In one or more embodiments, the PAP variant further comprises one or more mutations at residues selected from: G71, Y87, A131, F143, H205, M244, P293, A320, Q334, and K337 of SEQ ID NO:1, or at functionally equivalent residues thereto.

In one or more embodiments, the PAP variant further comprises one or more mutations at residues selected from: G71, A131, H205, M244, P293, Q334, and K337 of SEQ ID NO:1, or at functionally equivalent residues thereto.

In one or more embodiments, the PAP variant further comprises one or more mutations at residues selected from: G71, Y87, F143, N195, H205, V240, P293, M318, and A320, or at functionally equivalent residues thereto.

In one or more embodiments, the PAP variant further comprises one or more mutations at residues selected from: Y87, F143, N195, V240, P293, M318, and A320 or at functionally equivalent residues thereto.

In one or more embodiments, the PAP variant further comprises one or more mutations at residues selected from: Y87, F143, P293, M318 and A320 or at functionally equivalent residues thereto.

In one or more embodiments, the PAP variant further comprises one or more mutations at residues selected from: Y87, F143, and P293 or at functionally equivalent residues thereto.

In one or more embodiments, the PAP variant further comprises a mutation at residue: P293 of SEQ ID NO:1, or at a functionally equivalent residue thereto.

In one or more embodiments, the PAP variant further comprises a mutation at residue: M244 of SEQ ID NO:1, or at a functionally equivalent residue thereto.

In one or more embodiments, the PAP variant further comprises one or more mutations at residues selected from: M244, and H205 of SEQ ID NO:1, or at functionally equivalent residues thereto.

In one or more embodiments, the PAP variant further comprises one or more mutations at residues selected from: M244, H205, and G71 of SEQ ID NO:1, or at functionally equivalent residues thereto.

In one or more embodiments, the PAP variant further comprises one or more mutations at residues selected from: M244, H205, G71, and P293 of SEQ ID NO:1, or at functionally equivalent residues thereto.

In one or more embodiments, the PAP variant further comprises one or more mutations at residues selected from: M244, H205, G71, P293, and A320 of SEQ ID NO:1, or at functionally equivalent residues thereto.

In one or more embodiments, the PAP variant further comprises one or more mutations at residues selected from: M244, H205, G71, P293, A320, and F143 of SEQ ID NO:1, or at functionally equivalent residues thereto.

In one or more embodiments, the PAP variant further comprises a truncation, preferentially a C-terminal truncation. In one or more embodiments, the PAP variant further comprises a flexible loop replacement. In one or more embodiments, the flexible loop replacement is an ancestral loop or a 2GS loop or a “HDGAR” loop. In one or more embodiments, the C-terminal truncation is up to residue 606 of SEQ ID NO:1 or a functionally equivalent residue thereto.

In one or more embodiments, the PAP variant further comprises one or more mutations at residues selected from: P208, A410, E574 and/or P643 of SEQ ID NO:1, or functionally equivalent residues thereto.

According to another aspect of the present invention, there is provided the use of a poly(A)polymerase variant according to the first aspect for synthesising a polynucleotide.

In one or more embodiments, the polynucleotide is RNA or DNA.

In one or more embodiments, the polynucleotide is DNA.

According to another aspect of the present invention, there is provided a method of synthesising a polynucleotide comprising a step of contacting a nucleic acid fragment with a poly(A) polymerase variant according to the first aspect and one or more nucleotides under suitable conditions to incorporate the or each nucleotide into the nucleic acid.

According to another aspect of the present invention, there is provided a method of synthesising a polynucleotide comprising
(a) providing a nucleic acid initiator;
(b) contacting the nucleic acid initiator with a poly(A) polymerase variant according to the first aspect and a protected nucleotide under suitable conditions to incorporate the protected nucleotide into the nucleic acid initiator and form an elongated nucleic acid fragment;
(c) deprotecting the elongated nucleic acid fragment;
(d) repeating steps (b) and (c) on the elongated nucleic acid fragment until the polynucleotide is formed.

According to another aspect of the present invention, there is provided a polynucleotide obtained by methods of the invention.

According to another aspect of the present invention, there is provided a composition comprising the poly(A) polymerase variant according to the first aspect.

In one or more embodiments, the composition is a buffer solution, suitably an elongation buffer solution.

According to a another aspect of the present invention, there is provided a kit comprising a poly(A)polymerase variant according to the first aspect or a composition of the present invention, and one or more reagents for synthesising a polynucleotide.

According to another aspect of the present invention, there is provided a nucleic acid encoding a poly(A) polymerase variant according to the first aspect.

According to another aspect of the present invention, there is provided a vector comprising a nucleic acid according to the present invention.

According to another aspect of the present invention, there is provided a host cell comprising a poly(A) polymerase variant according to the first aspect, a nucleic acid according to the present invention, or a vector according to the present invention.

The invention described herein is a poly(A) polymerase variant comprising one or more targeted mutations, which may be used in combination, to modify the enzyme and provide improvements which are beneficial for its use in polynucleotide synthesis methods. The inventors have found that certain mutations can provide the enzyme with enhanced stability, activity, and increased purity of the polynucleotide product. The enhancement in activity is believed to be caused by a faster rate of incorporation of modified nucleotides during template-free polynucleotide synthesis, believed to be around 10× faster than previous polymerases used in the field. This leads to a quicker elongation/deprotection cycle time, and an increase in overall efficiency of the synthesis process. It is also believed that the higher activity and stability of the enzyme enables the synthesis of longer polynucleotide products than those produced by previous synthesis methods using other polymerases. Longer polynucleotides, such as those over 40 nucleotides, are useful for applications such as CRISPR-Cas probes. Therefore, the enzymes of the invention can be used to produce a wider variety of polynucleotide lengths. Furthermore, the purity of the polynucleotide products when using the enzymes of the invention is higher than obtained in previous methods. The inventors have demonstrated that the product is up to 30% more pure than polynucleotides producing using other polymerases. The higher stability of the enzyme means it can tolerate higher temperatures during synthesis reactions, which further leads to an increase in purity of ‘difficult’ sequences which contain a high GC content. The inventors have shown an increase of 10% purity of such ‘difficult’ sequences. This further expands the variety of polynucleotides that the enzymes of the invention can be used to produce. Therefore, the invention provides a much improved polymerase enzyme for use in template-free polynucleotide synthesis methods.

Further features and embodiments of the above defined aspects will now be described under each of the following headed sections in the detailed description. The headed sections are not limiting, any feature in any of the sections may be combined with any aspect or embodiment herein in any workable combination.

### DETAILED DESCRIPTION OF THE INVENTION

### Poly(A) Polymerase Variant

The invention relates to poly(A) polymerase variant or a functional fragment thereof having one or more modifications resulting in improved PAP yield, PAP activity, PAP stability and improved purity of polynucleotides synthesised therefrom.

In one or more embodiments, the poly(A) polymerases may be known as PAPs herein.

In one or more embodiments, in accordance with the invention, the PAP variant comprises an amino acid sequence having at least 70% identity to SEQ ID NO:1 or to a functional fragment thereof and comprising mutations at residues V199, V240, and M318 of SEQ ID NO:1 or at functionally equivalent residues thereto.

According to another aspect of the present invention, there is provided a poly(A) polymerase (PAP) variant, or a functional fragment thereof, comprising an amino acid sequence having at least 70% identity to SEQ ID NO:1, and comprising mutations at residues V199, V240, and M318 of SEQ ID NO:1 or at functionally equivalent residues thereto.

According to another aspect of the present invention, there is provided a poly(A) polymerase (PAP) variant, or a functional fragment thereof, comprising an amino acid sequence having:
at least 70% identity to a functional fragment of SEQ ID NO:1,

and
at least 70% identity to a SEQ ID NO:1,

and comprising mutations at residues V199, V240, and M318 of SEQ ID NO:1 or at functionally equivalent residues thereto.

Optionally the PAP variant may comprise one or more further mutations in addition to those identified above. In one or more embodiments, the PAP variant further comprises one or more mutations at residues selected from: G71, Y87, A131, F143, N195, H205, P208, M244, P293, A320, Q334, K337, A410, E574, and P643 of SEQ ID NO:1, or at functionally equivalent residues thereto.

In one or more embodiments, the PAP variant further comprises one or more mutations at residues selected from: G71, Y87, F143, N195, H205, P208, M244, P293, A320, A410, E574, and P643 of SEQ ID NO:1, or at functionally equivalent residues thereto.

In one or more embodiments, the PAP variant further comprises one or more mutations at residues selected from: R41, G71, Y87, A131, F143, N195, H205, P208, M244, V289, P293, M318, A320, Q334, K337, E341, K381, S387, I401, A410, K415, M571, E574, E577 and P643 of SEQ ID NO:1, or at functionally equivalent residues thereto.

In one or more embodiments, the PAP variant further comprises one or more mutations at residues selected from: R41, G71, Y87, A131, F143, N195, H205, P208, M244, V289, P293, M318, A320, E341, K381, S387, I401, A410, K415, M571, E574, E577 and P643 of SEQ ID NO:1, or at functionally equivalent residues thereto.

In one or more embodiments, the PAP variant further comprises one or more mutations at residues wherein the enzyme further comprises a mutation at residues selected from R41, V289, E341, K381, S387, I401, K415, M571 and E577 of SEQ ID NO:1: In one or more embodiments, the PAP variant further comprises one or more mutations at residues selected from: G71, Y87, A131, F143, N195, H205, P208, M244, P293, A320, Q334, and K337 of SEQ ID NO:1, or at functionally equivalent residues thereto.

In one or more embodiments, the PAP variant further comprises one or more mutations at residues selected from: G71, Y87, A131, F143, H205, M244, P293, A320, Q334, and K337 of SEQ ID NO:1, or at functionally equivalent residues thereto.

In one or more embodiments, the PAP variant further comprises one or more mutations at residues selected from: G71, A131, H205, M244, P293, Q334, and K337 of SEQ ID NO:1, or at functionally equivalent residues thereto.

In one or more embodiments, the PAP variant further comprises one or more mutations at residues selected from: G71, Y87, F143, N195, H205, V240, P293, M318, and A320, or at functionally equivalent residues thereto.

In one or more embodiments, the PAP variant further comprises one or more mutations at residues selected from: Y87, F143, N195, V240, P293, M318, and A320 or at functionally equivalent residues thereto.

In one or more embodiments, the PAP variant further comprises one or more mutations at residues selected from: Y87, F143, P293, M318 and A320 or at functionally equivalent residues thereto.

In one or more embodiments, the PAP variant further comprises one or more mutations at residues selected from: Y87, F143, and P293 or at functionally equivalent residues thereto.

In one or more embodiments, the PAP variant further comprises a mutation at residue: P293 of SEQ ID NO:1, or a functionally equivalent residue thereto.

Preferentially, the PAP variant further comprises a mutation at residue: M244 of SEQ ID NO:1, or a functionally equivalent residue thereto.

In one or more embodiments, the PAP variant further comprises one or more mutations at residues selected from: M244, and H205 of SEQ ID NO:1, or at functionally equivalent residues thereto.

In one or more embodiments, the PAP variant further comprises one or more mutations at residues selected from: M244, H205, and G71 of SEQ ID NO:1, or at functionally equivalent residues thereto.

In one or more embodiments, the PAP variant further comprises one or more mutations at residues selected from: M244, H205, G71, and P293 of SEQ ID NO:1, or at functionally equivalent residues thereto.

In one or more embodiments, the PAP variant further comprises one or more mutations at residues selected from: M244, H205, G71, P293, and A320 of SEQ ID NO:1, or at functionally equivalent residues thereto.

In one or more embodiments, the PAP variant further comprises one or more mutations at residues selected from: M244, H205, G71, P293, A320, and F143 of SEQ ID NO:1, or at functionally equivalent residues thereto.

In one or more embodiments, the PAP variant may further comprise a modification to the flexible loop region. The expression “flexible loop region” has its usual meaning in the art. In particular, it corresponds to residues 420 to 539 of SEQ ID NO:1 or functionally equivalent residues thereto.

In one or more embodiments, the PAP variant may further comprise a modification to the C-terminal region.

Optionally the modification in both instances may be a deletion, substitution, replacement, insertion, truncation or the like.

Preferentially, the modification to the flexible loop region is a truncation or a replacement, preferentially the modification to the C-terminal region is a C-terminal truncation. In one or more embodiments, the PAP variant may comprise both a flexible loop replacement and a C-terminal truncation.

Preferentially, the flexible loop modification is a replacement, preferentially wherein the flexible loop is replaced with an ancestral loop (such as those set forth in SEQ ID NO:32 and SEQ ID NO:33) or a 2GS sequence (SEQ ID NO:31) or a HDGAR sequence (SEQ ID NO:157).

Preferentially the flexible 2GS loop may comprise the sequence: GGGSGGGS (SEQ ID NO:31). Preferentially the flexible 2GS loop may consist in the sequence: GGGSGGGS (SEQ ID NO:31) Preferentially, the flexible loop may comprise the sequence HDGAR (SEQ ID NO: 157).

Even more preferentially, the flexible loop may consist in the sequence HDGAR (SEQ ID NO:157).

Preferentially, the C-terminal truncation is up to residue 606 of SEQ ID NO:1 or a functionally equivalent residue thereto. In other words, the residues corresponding to residues 607 to 650 of SEQ ID NO:1 or functionally equivalent residues thereto are truncated (absent). In other embodiments, the C-terminal truncation is up to residue 640, preferentially 635, even more preferentially 630, 625, 620, 615 or 610 of SEQ ID NO:1 or a functionally equivalent residue thereto. In other embodiments, the C-terminal truncation is up to residue 605, preferentially 604, even more preferentially 603, 602, 601, 600; 599, 598, 597, 596, 595, 594, 593, 592, 591, 590, 589, 588, 587, 586, 585, 584, 583, 582, 581, 580 or 579 of SEQ ID NO:1 or a functionally equivalent residue thereto.

Optionally, the PAP variant may further comprise a modification to the N-terminal region, such as a N-terminal truncation. Typically, the N-terminal truncation can be up to residue 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 of SEQ ID NO:1 of a functionally equivalent residue thereto.

Without wishing to be bound by theory, the inventors believe that a C-terminal truncation and/or a flexible loop replacement can lead to increased production yields of the PAP variant.

In one or more embodiments, the PAP variants may further comprise one or more mutations at residues selected from: P208, A410, E574 and/or P643 of SEQ ID NO:1, or at functionally equivalent residues thereto.

In one or more embodiments the PAP variant comprises at least 70%, at least 75%, at least 80%, at least 85%, at least 90%, at least 95%, at least 96%, at least 97%, at least 98%, at least 99% identity to SEQ ID NO:1. However suitably the PAP variant retains the mutations listed, or mutations at functionally equivalent residues.

In one or more embodiments the PAP variant comprises mutations at residues V199, V240, and M318 of SEQ ID NO:1, or at functionally equivalent residues thereto. Optionally further comprising mutations at residues A410 and P643 of SEQ ID NO:1, or at functionally equivalent residues thereto. Preferentially, the PAP variant comprises at least 70% identity to SEQ ID NO:3. More preferentially, the PAP variant consists of SEQ ID NO: 3.

In one or more embodiments the PAP variant comprises mutations at residues P208, V240, and M318 of SEQ ID NO:1, or at functionally equivalent residues thereto. Optionally further comprising mutations at residues A410 and P643 of SEQ ID NO:1, or at functionally equivalent residues thereto. Preferentially, the PAP variant comprises at least 70% identity to SEQ ID NO:4. More preferentially, the PAP variant consists of SEQ ID NO: 4.

In one or more embodiments the PAP variant comprises mutations at residues V240, M244 and M318 of SEQ ID NO:1, or at functionally equivalent residues thereto. Optionally further comprising mutations at residues A410 and P643 of SEQ ID NO:1, or at functionally equivalent residues thereto. Preferentially, the PAP variant comprises at least 70% identity to SEQ ID NO:5. More preferentially, the PAP variant consists of SEQ ID NO: 5.

In one or more embodiments the PAP variant comprises mutations at residues V199, P208, V240, M244, and M318, of SEQ ID NO:1, or at functionally equivalent residues thereto. Optionally further comprising mutations at residues A410 and P643 of SEQ ID NO:1, or at functionally equivalent residues thereto. Preferentially, the PAP variant comprises at least 70% identity to SEQ ID NO:6. More preferentially, the PAP variant consists of SEQ ID NO: 6.

In one or more embodiments the PAP variant comprises mutations at residues V199, P208, V240, M244, P293 and M318 of SEQ ID NO:1, or at functionally equivalent residues thereto. Optionally further comprising mutations at residues A410 and P643 of SEQ ID NO:1, or at functionally equivalent residues thereto. Preferentially, the PAP variant comprises at least 70% identity to SEQ ID NO:7. More preferentially, the PAP variant consists of SEQ ID NO: 7.

In one or more embodiments the PAP variant comprises mutations at residues G71, V199, P208, V240, M244, and M318 of SEQ ID NO:1, or at functionally equivalent residues thereto. Optionally further comprising mutations at residues A410 and P643 of SEQ ID NO:1, or at functionally equivalent residues thereto. Preferentially, the PAP variant comprises at least 70% identity to SEQ ID NO:8. More preferentially, the PAP variant consists of SEQ ID NO: 8.

In one or more embodiments the PAP variant comprises mutations at residues V199, H205, P208, V240, M244, and M318 of SEQ ID NO:1, or at functionally equivalent residues thereto. Optionally further comprising mutations at residues A410 and P643 of SEQ ID NO:1, or at functionally equivalent residues thereto. Preferentially, the PAP variant comprises at least 70% identity to SEQ ID NO:9. More preferentially, the PAP variant consists of SEQ ID NO: 9.

In one or more embodiments the PAP variant comprises mutations at residues G71, V199, H205, P208, V240, M244, P293 and M318 of SEQ ID NO:1, or at functionally equivalent residues thereto. Optionally further comprising mutations at residues A410, E574, and P643 of SEQ ID NO:1, or at functionally equivalent residues thereto. Preferentially, the PAP variant comprises at least 70% identity to SEQ ID NO:10. More preferentially, the PAP variant consists of SEQ ID NO: 10.

In one or more embodiments the PAP variant comprises mutations at residues G71, A131, V199, H205, P208, V240, M244, P293 and M318 of SEQ ID NO:1, or at functionally equivalent residues thereto. Optionally further comprising mutations at residues A410, and P643 of SEQ ID NO:1, or at functionally equivalent residues thereto. Preferentially, the PAP variant comprises at least 70% identity to/consists of SEQ ID NO:11. More preferentially, the PAP variant consists of SEQ ID NO: 11.

In one or more embodiments the PAP variant comprises mutations at positions G71, A131, V199, H205, P208, V240, M244, P293, M318, Q334, and K337 of SEQ ID NO:1, or at functionally equivalent residues thereto. Optionally further comprising mutations at residues A410, and P643 of SEQ ID NO:1, or at functionally equivalent residues thereto. Preferentially, the PAP variant comprises at least 70% identity to SEQ ID NO:12. More preferentially, the PAP variant consists of SEQ ID NO: 12.

In one or more embodiments the PAP variant comprises mutations at residues V199, H205, P208, V240, M244, M318 and A320 of SEQ ID NO:1, or at functionally equivalent residues thereto. Optionally further comprising mutations at residues A410, and P643 of SEQ ID NO:1, or at functionally equivalent residues thereto. Preferentially, the PAP variant comprises at least 70% identity to/consists of SEQ ID NO:13. More preferentially, the PAP variant consists of SEQ ID NO: 13.

In one or more embodiments the PAP variant comprises mutations at residues N195, V199, H205, P208, V240, M244, and M318 of SEQ ID NO:1, or at functionally equivalent residues thereto. Optionally further comprising mutations at residues A410 and P643 of SEQ ID NO:1, or at functionally equivalent residues thereto. Preferentially, the PAP variant comprises at least 70% identity to SEQ ID NO:14. More preferentially, the PAP variant consists of SEQ ID NO: 14.

In one or more embodiments the PAP variant comprises mutations at residues V199, H205, P208, M244, M318, and A320, or at functionally equivalent residues thereto. Optionally further comprising mutations at residues A410 and P643 of SEQ ID NO:1, or at functionally equivalent residues thereto. Preferentially, the PAP variant comprises at least 70% identity to SEQ ID NO:15. More preferentially, the PAP variant consists of SEQ ID NO: 15.

In one or more embodiments the PAP variant comprises mutations at residues G71, F143, V199, H205, P208, V240, M244, P293, and M318 of SEQ ID NO:1, or at functionally equivalent residues thereto. Optionally further comprising mutations at residues A410, E574, and P643 of SEQ ID NO:1, or at functionally equivalent residues thereto. Preferentially, the PAP variant comprises at least 70% identity to SEQ ID NO:116. More preferentially, the PAP variant consists of SEQ ID NO: 16.

In one or more embodiments the PAP variant comprises mutations at residues G71, V199, H205, P208, V240, M244, P293, M318, and A320 of SEQ ID NO:1, or at functionally equivalent residues thereto. Optionally further comprising mutations at residues A410, E574, and P643 of SEQ ID NO:1, or at functionally equivalent residues thereto. Preferentially, the PAP variant comprises at least 70% identity to/consists of SEQ ID NO:17. More preferentially, the PAP variant consists of SEQ ID NO: 17.

In one or more embodiments the PAP variant comprises mutations at residues G71, F143, V199, H205, P208, V240, M244, P293, M318 and A320 of SEQ ID NO:1, or at functionally equivalent residues thereto. Optionally further comprising mutations at residues A410, E574, and P643 of SEQ ID NO:1, or at functionally equivalent residues thereto. Preferentially, the PAP variant comprises at least 70% identity to SEQ ID NO:18. More preferentially, the PAP variant consists of SEQ ID NO: 18.

In one or more embodiments the PAP variant comprises: mutations at residues G71, F143, N195, V199, H205, P208, V240, M244, P293, M318, and A320 of SEQ ID NO:1, or at functionally equivalent residues thereto. Optionally further comprising mutations at residues A410, E574, and P643 of SEQ ID NO:1, or at functionally equivalent residues thereto. Preferentially, the PAP variant comprises at least 70% identity to SEQ ID NO:19. More preferentially, the PAP variant consists of SEQ ID NO: 19.

In one or more embodiments the PAP variant comprises: mutations at residues G71, F143, V199, H205, P208, V240, M244, P293, M318, and A320 of SEQ ID NO:1, or at functionally equivalent residues thereto; an ancestral flexible loop replacement; and a C-terminal truncation. Optionally further comprising a mutation at residue A410 of SEQ ID NO:1, or at a functionally equivalent residue thereto. Preferentially the flexible loop comprises the sequence: TNVKPEPHDDEAKVKLEDIPEKEAQPED (SEQ ID NO:32). More preferentially the flexible loop consists of the sequence TNVKPEPHDDEAKVKLEDIPEKEAQPED (SEQ ID NO:32). Preferentially the C terminal truncation comprises a truncation up to residue 606 of SEQ ID NO:1, or a functionally equivalent residue thereto. Preferentially, the PAP variant comprises at least 70% identity to SEQ ID NO:20. More preferentially, the PAP variant consists of SEQ ID NO: 20.

In one or more embodiments the PAP variant comprises: mutations at residues G71, F143, V199, H205, P208, V240, M244, P293, M318, and A320 f SEQ ID NO:1, or at functionally equivalent residues thereto; a 2GS flexible loop replacement; and a C-terminal truncation. Optionally further comprising a mutation at residue A410 of SEQ ID NO:1, or a functionally equivalent residue thereto. Preferentially the flexible loop comprises the sequence: GGGSGGGS (SEQ ID NO:31). More preferentially the flexible loop consists of the sequence GGGSGGGS (SEQ ID NO:31). Preferentially the C terminal truncation comprises a truncation of up to residue 606 of SEQ ID NO:1, or a functionally equivalent residue thereto. Preferentially, the PAP variant comprises at least 70% identity to SEQ ID NO:21. More preferentially, the PAP variant consists of SEQ ID NO: 21.

In one or more embodiments the PAP variant comprises: mutations at residues G71, Y87, F143, V199, H205, P208, V240, M244, P293, M318, and A320 of SEQ ID NO:1, or at functionally equivalent residues thereto. Optionally further comprising mutations at residues A410, E574, and P643 of SEQ ID NO:1, or at functionally equivalent residues thereto. In such an embodiment, the PAP variant preferentially comprises at least 70% identity to SEQ ID NO:22.

Optionally this variant further comprises mutations at residues A410, and P643 of SEQ ID NO:1, or at functionally equivalent residues thereto. In such an embodiment, the PAP variant preferentially comprises at least 70% identity to SEQ ID NO:23.

Optionally this PAP variant further comprises mutations at residues E574, and P643 of SEQ ID NO:1, or at functionally equivalent residues thereto. In such an embodiment, the PAP variant preferentially comprises at least 70% identity to SEQ ID NO:24.

Optionally this PAP variant further comprises mutations at residues A410, and E574 of SEQ ID NO:1, or at functionally equivalent residues thereto. In such an embodiment, the PAP variant preferentially comprises at least 70% identity to SEQ ID NO:25.

More preferentially, the PAP variant consists of SEQ ID NO: 22. Preferentially, the PAP variant consists of SEQ ID NO: 23. More preferentially, the PAP variant consists of SEQ ID NO: 24. More preferentially, the PAP variant consists of SEQ ID NO: 25.

In one or more embodiments the PAP variant comprises: mutations at residues G71, Y87, F143, V199, H205, V240, M244, P293, M318, and A320 of SEQ ID NO:1, or at functionally equivalent residues thereto. Optionally further comprising mutations at residues A410, E574, and P643 of SEQ ID NO:1, or at functionally equivalent residues thereto. In such an embodiment, the PAP variant preferentially comprises at least 70% identity to SEQ ID NO:26. More preferentially, the PAP variant consists of SEQ ID NO: 26.

Optionally, in relation to any of sequence ID NOs 22 to 26, the flexible loop may comprise the sequence: FGDEKVKSETKSEVKQEVKQEVRQDDVIQDGVPVKQEKAEVRAEDGVRIKRELSEEVQLP PPTNVKPEPHDDEAKVKLEDIPEKEAQPED (SEQ ID NO:33). Optionally the flexible loop may consist of the sequence FGDEKVKSETKSEVKQEVKQEVRQDDVIQDGVPVKQEKAEVRAEDGVRIKRELSEEVQLP PPTNVKPEPHDDEAKVKLEDIPEKEAQPED (SEQ ID NO:33). Optionally the C-terminal may comprise a truncation, optionally the C terminal truncation comprises a truncation up to residue 606 of SEQ ID NO:1, or a functionally equivalent residue thereto.

In one or more embodiments the PAP variant comprises: mutations at residues G71, F143, V199, H205, V240, M244, P293, M318, and A320 of SEQ ID NO:1, or a functionally equivalent residue; a 2GS flexible loop replacement; and a C-terminal truncation. Optionally further comprising a mutation at residue A410 of SEQ ID NO:1, or a functionally equivalent residue thereto. Preferentially the flexible loop comprises the sequence: GGGSGGGS (SEQ ID NO:31). More preferentially the flexible loop consists of the sequence GGGSGGGS (SEQ ID NO:31). Preferentially the C terminal truncation comprises a truncation up to residue 606 of SEQ ID NO:1, or a functionally equivalent residue thereto. Preferentially, the PAP variant comprises at least 70% identity to SEQ ID NO: 27. More preferentially, the PAP variant consists of SEQ ID NO: 27.

In one or more embodiments the PAP variant comprises: mutations at residues G71, Y87, F143, V199, H205, V240, M244, P293, M318, and A320 of SEQ ID NO:1, or at functionally equivalent residues thereto; a 2GS flexible loop replacement; and a C-terminal truncation. Optionally further comprising a mutation at residue A410 of SEQ ID NO:1, or a functionally equivalent residue thereto. Preferentially the flexible loop comprises the sequence: GGGSGGGS (SEQ ID NO:31). More preferentially the flexible loop consists of the sequence GGGSGGGS (SEQ ID NO:31). Preferentially the C terminal truncation comprises a truncation up to residue 606 of SEQ ID NO:1, or a functionally equivalent residue thereto. Preferentially, the PAP variant comprises at least 70% identity to SEQ ID NO: 28. More preferentially, the PAP variant consists of SEQ ID NO: 28.

Optionally in any of the embodiments described above, the PAP variant may not comprise a mutation at residue E574, or a functionally equivalent residue thereto. Optionally in any of the embodiments described above, the PAP variant may not comprise a mutation at residue A410, or a functionally equivalent residue thereto. Optionally in any of the embodiments described above, the PAP variant may not comprise a mutation at position P643, or a functionally equivalent residue thereto. Optionally in any of the embodiments described above, the PAP variant may not comprise a mutation at residue P208, or a functionally equivalent residue thereto.

Most preferentially, the PAP variant comprises or consists of SEQ ID NO:26 or 28.

In one or more embodiments the PAP variant comprises at least 70% identity to any one of sequences SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121 or SEQ ID NO: 122. More preferentially, the PAP variant consists of SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121 or SEQ ID NO: 122.

In one or more embodiments the PAP variant comprises: mutations at residues G71, Y87, F143, V199, H205, V240, M244, P293, M318, and A320 of SEQ ID NO:1, or at functionally equivalent residues thereto; a “HDGAR” flexible loop replacement; and a C-terminal truncation. Optionally further comprising a mutation at residue A410 of SEQ ID NO:1, or a functionally equivalent residue thereto. Preferentially the flexible loop comprises the sequence: HDGAR (SEQ ID NO:157). More preferentially the flexible loop consists of the sequence HDGAR (SEQ ID NO:157). Preferentially the C terminal truncation comprises a truncation up to residue 606 of SEQ ID NO:1, or a functionally equivalent residue thereto. Preferentially, the PAP variant comprises at least 70% identity to SEQ ID NO: 123. More preferentially, the PAP variant consists of SEQ ID NO: 123.

In one or more embodiments the PAP variant comprises: mutations at residues G71, Y87, F143, V199, H205, V240, M244, P293, M318, A320 and I401 of SEQ ID NO:1, or at functionally equivalent residues thereto; a 2GS flexible loop replacement; and a C-terminal truncation. Preferentially the flexible loop comprises the sequence: GGGSGGGS (SEQ ID NO:31). More preferentially the flexible loop consists of the sequence GGGSGGGS (SEQ ID NO:31). Preferentially the C terminal truncation comprises a truncation up to residue 606 of SEQ ID NO:1, or a functionally equivalent residue thereto. Preferentially, the PAP variant comprises at least 70% identity to SEQ ID NO: 124. More preferentially, the PAP variant consists of SEQ ID NO: 124.

In one or more embodiments the PAP variant comprises: mutations at residues G71, Y87, F143, V199, H205, V240, M244, P293, M318, A320 and K 381 of SEQ ID NO:1, or at functionally equivalent residues thereto; a 2GS flexible loop replacement; and a C-terminal truncation. Preferentially the flexible loop comprises the sequence: GGGSGGGS (SEQ ID NO:31). More preferentially the flexible loop consists of the sequence GGGSGGGS (SEQ ID NO:31). Preferentially the C terminal truncation comprises a truncation up to residue 606 of SEQ ID NO:1, or a functionally equivalent residue thereto. Preferentially, the PAP variant comprises at least 70% identity to SEQ ID NO: 125. More preferentially, the PAP variant consists of SEQ ID NO: 125.

In one or more embodiments the PAP variant comprises: mutations at residues G71, Y87, F143, V199, H205, V240, M244, P293, M318, A320 and S387 of SEQ ID NO:1, or at functionally equivalent residues thereto; a 2GS flexible loop replacement; and a C-terminal truncation. Preferentially the flexible loop comprises the sequence: GGGSGGGS (SEQ ID NO:31). More preferentially the flexible loop consists of the sequence GGGSGGGS (SEQ ID NO:31). Preferentially the C terminal truncation comprises a truncation up to residue 606 of SEQ ID NO:1, or a functionally equivalent residue thereto. Preferentially, the PAP variant comprises at least 70% identity to SEQ ID NO: 125. More preferentially, the PAP variant consists of SEQ ID NO: 126.

In one or more embodiments the PAP variant comprises: mutations at residues R41, G71, Y87, A131, F143, V199, H205, V240, M244, V289, P293, M318, A320 and E341 of SEQ ID NO:1, or at functionally equivalent residues thereto; a “HDGAR” flexible loop replacement; and a C-terminal truncation. Preferentially the flexible loop comprises the sequence: HDGAR (SEQ ID NO:157). More preferentially the flexible loop consists of the sequence HDGAR (SEQ ID NO:157). Preferentially the C terminal truncation comprises a truncation up to residue 606 of SEQ ID NO:1, or a functionally equivalent residue thereto. Preferentially, the PAP variant comprises at least 70% identity to SEQ ID NO: 127. More preferentially, the PAP variant consists of SEQ ID NO: 127.

In one or more embodiments the PAP variant comprises: mutations at residues R41, G71, Y87, A131, F143, V199, H205, V240, M244, V289, P293, M318, A320, E341, S387, 1401 and M571 of SEQ ID NO:1, or at functionally equivalent residues thereto; a “HDGAR” flexible loop replacement; and a C-terminal truncation. Preferentially the flexible loop comprises the sequence: HDGAR (SEQ ID NO:157). More preferentially the flexible loop consists of the sequence HDGAR (SEQ ID NO:157). Preferentially the C terminal truncation comprises a truncation up to residue 606 of SEQ ID NO:1, or a functionally equivalent residue thereto. Preferentially, the PAP variant comprises at least 70% identity to SEQ ID NO: 127. More preferentially, the PAP variant consists of SEQ ID NO: 128.

In one or more embodiments the PAP variant comprises: mutations at residues R41, G71, Y87, A131, F143, V199, H205, V240, M244, V289, P293, M318, A320, E341, S387, 1401, K415 and M571 of SEQ ID NO:1, or at functionally equivalent residues thereto; a “HDGAR” flexible loop replacement; and a C-terminal truncation. Preferentially the flexible loop comprises the sequence: HDGAR (SEQ ID NO:157. More preferentially the flexible loop consists of the sequence HDGAR (SEQ ID NO:157). Preferentially the C terminal truncation comprises a truncation up to residue 606 of SEQ ID NO:1, or a functionally equivalent residue thereto. Preferentially, the PAP variant comprises at least 70% identity to SEQ ID NO: 129. More preferentially, the PAP variant consists of SEQ ID NO: 129.

In one or more embodiments the PAP variant comprises: mutations at residues R41, G71, Y87, A131, F143, V199, H205, V240, M244, V289, P293, M318, A320, E341, K381, S387, I401, K415, M571 and E577 of SEQ ID NO:1, or at functionally equivalent residues thereto; a “HDGAR” flexible loop replacement; and a C-terminal truncation. Preferentially the flexible loop comprises the sequence: HDGAR (SEQ ID NO:157). More preferentially the flexible loop consists of the sequence HDGAR (SEQ ID NO:157. Preferentially the C terminal truncation comprises a truncation up to residue 606 of SEQ ID NO:1, or a functionally equivalent residue thereto. Preferentially, the PAP variant comprises at least 70% identity to SEQ ID NO: 130. More preferentially, the PAP variant consists of SEQ ID NO: 130.

In one or more embodiments, any of the above mutations are substitution mutations. A “substitution” means that an amino acid residue is replaced by another amino acid residue. In one or more embodiments, any amino acid may be used for the substitution. In one or more embodiments any proteinogenic amino acid may be used for the substitution. Preferentially the substitution is a conservative substitution.

By ‘conservative’ it is meant that an amino acid with similar characteristics may be used for the substitution. Conservative amino acid substitutions” refer to the interchangeability of residues having similar side chains, and thus typically involves substitution of an amino acid in a polypeptide with amino acids within the same or similar defined class of amino acids. By way of example, an amino acid with an aliphatic side chain may be substituted with another aliphatic amino acid, e.g., alanine, valine, leucine, and isoleucine; an amino acid with hydroxyl side chain may be substituted with another amino acid with a hydroxyl side chain, e.g., serine and threonine; an amino acids having aromatic side chains may be substituted with another amino acid having an aromatic side chain, e.g., phenylalanine, tyrosine, tryptophan, and histidine; an amino acid with a basic side chain may be substituted with another amino acid with a basic side chain, e.g., lysine and arginine; an amino acid with an acidic side chain may be substituted with another amino acid with an acidic side chain, e.g., aspartic acid or glutamic acid; and a hydrophobic or hydrophilic amino acid may be substituted with another hydrophobic or hydrophilic amino acid, respectively.

In one or more embodiments the mutation at residue G71 or a functionally equivalent residue thereto is G71P. In one or more embodiments the mutation at residue Y87 or a functionally equivalent residue thereto is Y87H. In one or more embodiments the mutation at residue A131 or a functionally equivalent residue thereto is A131G. In one or more embodiments the mutation at residue F143 or a functionally equivalent residue thereto is F143Y. In one or more embodiments the mutation at residue N195 or a functionally equivalent residue thereto is N195S. In one or more embodiments the mutation at residue V199 or a functionally equivalent residue thereto is V199N or V199T. In one or more embodiments the mutation at residue H205 or a functionally equivalent residue thereto is H205R or H205K. In one or more embodiments the mutation at residue P208 or a functionally equivalent residue thereto is P208H. In one or more embodiments the mutation at residue V240 or a functionally equivalent residue thereto is V240A. In one or more embodiments the mutation at residue M244 or a functionally equivalent residue thereto is M244V. In one or more embodiments the mutation at residue P293 or a functionally equivalent residue thereto is P293R. In one or more embodiments the mutation at residue M318 or a functionally equivalent residue thereto is M318T. In one or more embodiments the mutation at residue A320 or a functionally equivalent residue thereto is A320T or A320C. In one or more embodiments the mutation at residue Q334 or a functionally equivalent residue thereto is Q334K. In one or more embodiments the mutation at residue K337 or a functionally equivalent residue thereto is K337E. In one or more embodiments the mutation at residue A410 or a functionally equivalent residue thereto is A410V. In one or more embodiments the mutation at residue E574 or a functionally equivalent residue thereto is E574K. In one or more embodiments the mutation at residue P643 or a functionally equivalent residue thereto is P643A. In one or more embodiments the mutation at residue R41 or a functionally equivalent residue thereto is R41P. In one or more embodiments the mutation at residue V289 or a functionally equivalent residue thereto is V289A. In one or more embodiments the mutation at residue E341 or a functionally equivalent residue thereto is E341 S. In one or more embodiments the mutation at residue K381 or a functionally equivalent residue thereto is K381Q. In one or more embodiments the mutation at residue S387 or a functionally equivalent residue thereto is S387R. In one or more embodiments the mutation at residue I401 or a functionally equivalent residue thereto is I401L. In one or more embodiments the mutation at residue K415 or a functionally equivalent residue thereto is K415Q. In one or more embodiments the mutation at residue M571 or a functionally equivalent residue thereto is M571K. In one or more embodiments the mutation at residue E577 or a functionally equivalent residue thereto is E577R.

The amino acids are herein represented by their one-letter or three-letters code according to the standard international nomenclature: A: alanine (Ala); C: cysteine (Cys); D: aspartic acid (Asp); E: glutamic acid (Glu); F: phenylalanine (Phe); G: glycine (Gly); H: histidine (His); I: isoleucine (He); K: lysine (Lys); L: leucine (Leu); M: methionine (Met); N: asparagine (Asn); P: proline (Pro); Q: glutamine (Gin); R: arginine (Arg); S: serine (Ser); T: threonine (Thr); V: valine (Vai); W: tryptophan (Trp) and Y: tyrosine (Tyr).

In one or more embodiments the PAP variant comprises at least 70%, at least 75%, at least 80%, at least 85%, at least 90%, at least 95%, at least 96%, at least 97%, at least 98%, at least 99% identity to SEQ ID NO:1 or to a functional fragment thereof. However the PAP variant retains the mutations listed, or mutations at a functionally equivalent residue to those listed, namely the mutations at residues V199, V240 and M318 of SEQ ID NO:1 or functionally equivalent residues thereto; and the optional further mutations. In one or more embodiments any amino acid sequence identified herein may have at least 70%, at least 75%, at least 80%, at least 85%, at least 90%, at least 95%, at least 96%, at least 97%, at least 98%, at least 99% identity to a reference amino acid sequence. In one or more embodiments any nucleic acid sequence identified herein may have at least 70%, at least 75%, at least 80%, at least 85%, at least 90%, at least 95%, at least 96%, at least 97%, at least 98%, at least 99% identity to a reference nucleic acid sequence.

In one or more embodiments the PAP variant comprises at least 70%, at least 75%, at least 80%, at least 85%, at least 90%, at least 95%, at least 96%, at least 97%, at least 98%, at least 99% identity to any of the modified sequences of SEQ ID NO:3, 6-28, 110-130, 142-145, 151-152, 155-156 and 158-159 identified herein. Preferentially the PAP variant may consist of any of the modified sequences SEQ ID NO:3, 6-28, 110-130 142-145, 151-152, 155-156 and 158-159 identified.

“Identity” or “percent identity” refers to the degree of sequence variation between two given nucleic acid or amino acid sequences. For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters. Optimal alignment of sequences for comparison can be conducted, e.g., by the local homology algorithm of (Smith and Waterman, 1981), by the homology alignment algorithm of (Needleman and Wunsch, 1970), by the search for similarity method of (Pearson and Lipman, 1988), by computerized implementations of these algorithms (GAP, BESTFIT, FASTA, and TFASTA in the Wisconsin Genetics Software Package, Genetics Computer Group, 575 Science Dr., Madison, WI), or by visual inspection. One example of an algorithm that is suitable for determining percent sequence identity and sequence similarity is the BLAST algorithm, which is described in (Altschul et al., 1990). Software for performing BLAST analyses is publicly available through the National Center for Biotechnology Information (on the world wide web at ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighbourhood word score threshold (Altschul et al., 1990) These initial neighbourhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix ((Henikoff and Henikoff, 1992). In addition to calculating percent sequence identity, the BLAST algorithm also performs a statistical analysis of the similarity between two sequences (Karlin and Altschul, 1990). One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleic acid sequence to the reference nucleic acid sequence is less than about 0.1, more preferably less than about 0.01, and most preferably less than about 0.001.

The sequence identity is determined by comparing the sequences when aligned so as to maximize overlap and identity while minimizing sequence gaps. In particular, sequence identity may be determined using any of a number of mathematical global or local alignment algorithms, depending on the length of the two sequences. Sequences of similar lengths are preferably aligned using a global alignment algorithm (e.g. Needleman and Wunsch algorithm; Needleman and Wunsch, 1970) which aligns the sequences optimally over the entire length, while sequences of substantially different lengths are preferably aligned using a local alignment algorithm (e.g. Smith and Waterman algorithm (Smith and Waterman, 1981) or Altschul algorithm (Altschul et al., 1997; Altschul et al., 2005)). Alignment for purposes of determining percent amino acid sequence identity can be achieved in various ways that are within the skill in the art, for instance, using publicly available computer software available on internet web sites such as http://blast.ncbi.nlm.nih.gov/ or http://www.ebi.ac.uk/Tools/emboss/. Those skilled in the art can determine appropriate parameters for measuring alignment, including any algorithm needed to achieve maximal alignment over the full length of the sequences being compared. For purposes herein, % amino acid sequence identity values refer to values generated using the pair wise sequence alignment program EMBOSS Needle, that creates an optimal global alignment of two sequences using the Needleman-Wunsch algorithm, wherein all search parameters are set to default values, i.e. Scoring matrix=BLOSUM62, Gap open=10, Gap extend=0.5, End gap penalty=false, End gap open=10 and End gap extend=0.5·‘Functionally equivalent residue’ as used herein means the amino acid residue having the same function (which may be the same residue) in a different reference sequence to that cited, suitably in a different poly(A) polymerase enzyme sequence to that cited. Therefore, whilst the statements herein refer to certain sequences by SEQ ID Nos, the invention is not restricted to poly(A) polymerase enzyme of said SEQ ID Nos, each modification listed may be located at a residue equivalent to an amino acid residue denoted above in another poly(A) polymerase enzyme sequence. By equivalent residue it is meant the residue that has the same function as the residue listed, which may often be the residue at the same or corresponding position to the residue listed, in a different poly(A) polymerase enzyme sequence. In some embodiments, a ‘functionally equivalent residue’ is at a ‘corresponding position’ to the position listed. Therefore, the term ‘functionally equivalent residue’ may be substituted with the term ‘corresponding position thereto’ at any instance herein, and the term ‘residue’ may be used interchangeably with the term ‘position’ herein. Therefore, in any embodiment herein, the PAP variant may comprise mutations/modifications at the ‘positions’ listed, or mutations/modifications at ‘corresponding positions thereto’.

Therefore, the invention equally refers to other poly(A) polymerase enzymes having different amino acid sequences with the same or equivalent modifications. It is possible to compare poly(A) polymerase polypeptides by sequence comparison and locate conserved regions that correspond to the amino acid residues/positions listed above. Sequence comparison to find equivalent or corresponding residues/positions may be carried out by aligning the amino acid sequences of two or more proteins, using an alignment program such as BLAST®. Methods for the alignment of sequences for comparison are well known in the art, such methods include GAP, BESTFIT, BLAST, FASTA and TFASTA. GAP uses the algorithm of Needleman and Wunsch ((1970) J Mol Biol 48: 443-453) to find the global (i.e. spanning the complete sequences) alignment of two sequences that maximizes the number of matches and minimizes the number of gaps. The BLAST algorithm (Altschul et al. (1990) J Mol Biol 215: 403-10) calculates percent sequence identity and performs a statistical analysis of the similarity between the two sequences. The software for performing BLAST analysis is publicly available through the National Centre for Biotechnology Information (NCBI). Homologues may readily be identified using, for example, the ClustalW multiple sequence alignment algorithm (version 1.83), with the default pairwise alignment parameters, and a scoring method in percentage. Global percentages of similarity and identity may also be determined using one of the methods available in the MatGAT software package (Campanella et al., BMC Bioinformatics. 2003 Jul. 10; 4:29. MatGAT: an application that generates similarity/identity matrices using protein or DNA sequences.). Minor manual editing may be performed to optimise alignment between conserved motifs, as would be apparent to a person skilled in the art. Furthermore, instead of using full-length sequences for the identification of homologues, specific domains may also be used. The sequence identity values may be determined over the entire nucleic acid or amino acid sequence or over selected domains or conserved motif(s), using the programs mentioned above using the default parameters. For local alignments, the Smith-Waterman algorithm is particularly useful (Smith T F, Waterman M S (1981) J. Mol. Biol 147(1); 195-7). In the present case, a functionally equivalent residue or corresponding position in a different poly(A) polymerase enzyme sequence may be found by aligning the amino acid sequence of said other poly(A) polymerase enzyme with SEQ ID NO 1, and locating the same amino acid position as those listed.

The terms “modified”, “mutant” and “variant” can be used interchangeably herein to refer to polypeptides derived from poly(A) polymerases, or derivatives or functional fragments of such poly(A) polymerases, and in particular from a poly(A) polymerase such as the poly(A) polymerase according to the sequence SEQ ID NO. 1, and comprising one or more modifications, namely a substitution, an insertion and/or a deletion at one or more residues/positions of said sequence, and having a poly(A) polymerase activity. The terms “modified”, “mutant” and “variant” therefore refer to poly(A) polymerase enzymes. The variants can be obtained by various techniques well known in the art. In particular, examples of techniques for modifying the DNA sequence coding for the wild-type proteins comprise, without being limited thereto, directed mutagenesis, random mutagenesis, and the construction of synthetic oligonucleotides.

The term “functional fragment” is understood to mean a fragment of a poly(A) polymerase enzyme exhibiting poly(A)polymerase activity. The fragment can comprise 100, 200, 300, 310, 320, 330, 340, 350, 360, 370, 380, 390, 400, 410, 420, 430, 440, 450, 460, 470, 480, 490, 500, 510, 520, 530 or more consecutive amino acids of a poly(A) polymerase, preferably of a poly(A) polymerase enzyme as defined herein. Preferably, the fragment comprises the minimum number of consecutive amino acids of a poly(A)polymerase consisting of the catalytic fragment of said enzyme.

In one embodiment, the functional fragment can consist of several domains, each consisting of one or more consecutive amino acids of a poly(A) polymerase, separated by a flexible loop.

Typically, the functional fragment of the PAP having the sequence set forth in SEQ ID NO:1 corresponds to residues 28 to 419 and 540 to 588, separated by a flexible loop, such as a “HDGAR” loop.

Without wishing to be bound by theory, the inventors have discovered and demonstrated that the PAP enzyme having the sequence SEQ ID NO:1 could be truncated at the following three locations, whilst maintaining poly(A) polymerase activity:
at the N-terminus, truncations up to residue L28 of the sequence SEQ ID NO:1 can be made;
replacement of the flexible loop corresponding to residues 420 to 539 of the sequence SEQ ID NO:1 by any of the flexible loops described above, typically the “HDGAR” loop (SEQ ID NO:157), can be made;
at the C-terminus, truncations up to residue L588 of the sequence SEQ ID NO:1 can be made.

Thus, in one embodiment, the functional fragment of SEQ ID NO:1 is the sequence set forth in SEQ ID NO:158. This corresponds to residues 28-419 and 540-588 of SEQ ID NO:1, separated by a “HDGAR” flexible loop (SEQ ID NO:157).

In another embodiment, the functional fragment of SEQ ID NO:1 is the sequence set forth in SEQ ID NO:159. This corresponds to residues 1-419 and 540-606 of SEQ ID NO:1, separated by a “HDGAR” flexible loop (SEQ ID NO:157).

Typically, in the case of a truncated sequence to be compared with a longer sequence, the percentage of identity can be calculated and expressed either with respect to the full length longer sequence, or with respect to the common portion when this common portion corresponds to the “functional fragment” of the reference sequence.

For example, if a sequence of 50 residues is to be compared with a sequence of 100 residues which contains the exact same sequence (and additional residues on the N-terminus and the C-terminus of said sequence), the sequence identity could be expressed as either “100% identity over the common portion” or as “50% identity over the entire full length sequence”. The skilled person will readily understand that both types of information can be meaningful, depending on the feature that is being examined.

In one or more embodiments, PAP variants for use with the invention may be derived from a poly(A) polymerase from any organism. Preferentially the PAP variant is derived from a poly(A) polymerase from a thermophilic organism. More preferentially the PAP variant is derived from a poly(A) polymerase from a thermophilic microorganism such as a bacterium or yeast. Examples of suitable PAP enzymes, from which a PAP variant may be produced, and some functionally equivalent residues/corresponding positions to the residues/positions to those listed above with reference to SEQ ID NO:1 may be found in the following table: Table 1: Suitable PAP enzymes for use in the invention

TABLE 1
Suitable PAP enzymes for use in the invention

Poly(A)
Polymerase

| source organism | Equivalent or Corresponding Residues/Positions to be Modified | | | | | | | | | |
|---|---|---|---|---|---|---|---|---|---|---|
| Thermothielavioides | 71 | 87 | 143 | 199 | 205 | 240 | 244 | 293 | 318 | 320 |
| terrestris NRRL | | | | | | | | | | |
| 8126 (SEQ ID | | | | | | | | | | |
| NO: 1) | | | | | | | | | | |
| (or XP_003655488 2 = | | | | | | | | | | |
| SEQ ID NO: 78) | | | | | | | | | | |
| (or SEQ ID 146 | | | | | | | | | | |
| with 6His Tag) | | | | | | | | | | |
| Thermothelomyces | 71 | 87 | 143 | 199 | 205 | 240 | 244 | 293 | 318 | 320 |
| thermophilus | | | | | | | | | | |
| ATCC 42464 (SEQ | | | | | | | | | | |
| ID NO: 77) | | | | | | | | | | |
| Staphylotrichum | 71 | 87 | 143 | 199 | 205 | 240 | 244 | 293 | 318 | 320 |
| longicolle (SEQ ID | | | | | | | | | | |
| NO: 100) | | | | | | | | | | |
| Chaetomium sp. | 71 | 87 | 143 | 199 | 205 | 240 | 244 | 293 | 318 | 320 |
| MPI-CAGE-AT- | | | | | | | | | | |
| 0009 (SEQ ID NO: | | | | | | | | | | |
| 99) | | | | | | | | | | |
| Chaetomium | 71 | 87 | 143 | 199 | 205 | 240 | 244 | 293 | 318 | 320 |
| globosum (SEQ ID | | | | | | | | | | |
| NO: 102) | | | | | | | | | | |
| Madurella | 71 | 87 | 143 | 199 | 205 | 240 | 244 | 293 | 318 | 320 |
| mycetomatis (SEQ | | | | | | | | | | |
| ID NO: 101) | | | | | | | | | | |
| Chaetomium sp. | 71 | 87 | 143 | 199 | 205 | 240 | 244 | 293 | 318 | 320 |
| MPI-SDFR-AT- | | | | | | | | | | |
| 0129 (SEQ ID | | | | | | | | | | |
| NO: 103) | | | | | | | | | | |
| Chaetomium | 72 | 88 | 144 | 200 | 206 | 241 | 245 | 294 | 319 | 321 |
| thermophilum var. | | | | | | | | | | |
| thermophilum DSM | | | | | | | | | | |
| 1495 (SEQ ID | | | | | | | | | | |
| NO: 76) | | | | | | | | | | |
| Podospora | ‘NA’ | 85 | 141 | 197 | 203 | 238 | 242 | 291 | 316 | 318 |
| anserina S mat+ | | | | | | | | | | |
| (SEQ ID NO: 107) | | | | | | | | | | |
| Neurospora | 73 | 89 | 145 | 201 | 207 | 242 | 246 | 295 | 320 | 322 |
| tetrasperma FGSC | | | | | | | | | | |
| 2508 (SEQ ID | | | | | | | | | | |
| NO: 104) | | | | | | | | | | |
| Neurospora crassa | 73 | 89 | 145 | 201 | 207 | 242 | 246 | 295 | 320 | 322 |
| OR74A (SEQ ID | | | | | | | | | | |
| NO: 105) | | | | | | | | | | |
| Podospora comata | ‘NA’ | 85 | 141 | 197 | 203 | 238 | 242 | 291 | 316 | 318 |
| (SEQ ID NO: 108) | | | | | | | | | | |
| Sordaria | 73 | 89 | 145 | 201 | 207 | 242 | 246 | 295 | 320 | 322 |
| macrospora k-hell | | | | | | | | | | |
| (SEQ ID NO: 106) | | | | | | | | | | |
| Chaetomium | 71 | 87 | 143 | 199 | 205 | 240 | 244 | 293 | ‘NA’ | ‘NA’ |
| globosum CBS | | | | | | | | | | |
| 148.51 (SEQ ID | | | | | | | | | | |
| NO: 90) | | | | | | | | | | |
| Trichoderma | 64 | 80 | 136 | 190 | 196 | 231 | 235 | 282 | 307 | 309 |
| citrinoviride (SEQ | | | | | | | | | | |
| ID NO: 95) | | | | | | | | | | |
| Magnaporthiopsis | 71 | 87 | 143 | 199 | 205 | 240 | 244 | 291 | 316 | 318 |
| poae ATCC 64411 | | | | | | | | | | |
| (SEQ ID NO: 83) | | | | | | | | | | |
| Sodiomyces | 71 | 87 | 143 | 199 | 205 | 240 | 244 | 291 | 316 | 318 |
| alkalinus F11 (SEQ | | | | | | | | | | |
| ID NO: 97) | | | | | | | | | | |
| Xylaria | 71 | 87 | 143 | 199 | 205 | 240 | 244 | 291 | 316 | 318 |
| flabelliformis (SEQ | | | | | | | | | | |
| ID NO: 89) | | | | | | | | | | |
| Scedosporium | ‘NA’ | 84 | 140 | 197 | 203 | 238 | 242 | 289 | 314 | 316 |
| apiospermum | | | | | | | | | | |
| (SEQ ID NO: 94) | | | | | | | | | | |
| Valsa sordida | 71 | 87 | 143 | 200 | 206 | 241 | 245 | 292 | 317 | 319 |
| (SEQ ID NO: 87) | | | | | | | | | | |
| Golovinomyces | ‘NA’ | 86 | 142 | 195 | 201 | 236 | 240 | 288 | 313 | 315 |
| cichoracearum | | | | | | | | | | |
| (SEQ ID NO: 85) | | | | | | | | | | |
| Aspergillus | ‘NA’ | 89 | 145 | 198 | 204 | 239 | 243 | 290 | 315 | 317 |
| thermomutatus | | | | | | | | | | |
| (SEQ ID NO: 96) | | | | | | | | | | |
| Schizosaccharomyces | ‘NA’ | 86 | 139 | 192 | 198 | 233 | 237 | 284 | 309 | 311 |
| octosporus | | | | | | | | | | |
| yFS286 (SEQ ID | | | | | | | | | | |
| NO: 92) | | | | | | | | | | |
| Drechslerella | ‘NA’ | 43 | 102 | 155 | 161 | 196 | 200 | 247 | 272 | 274 |
| brochopaga (SEQ | | | | | | | | | | |
| ID NO: 82) | | | | | | | | | | |
| Lachancea | ‘NA’ | 87 | 140 | 193 | 199 | 234 | 238 | 285 | 310 | 312 |
| thermotolerans | | | | | | | | | | |
| CBS 6340 (SEQ ID | | | | | | | | | | |
| NO: 91) | | | | | | | | | | |
| Exophiala spinifera | ‘NA’ | 86 | 142 | 196 | 202 | 237 | 241 | 292 | 317 | 319 |
| (SEQ ID NO: 93) | | | | | | | | | | |
| Pyronema | ‘NA’ | 88 | 142 | 196 | 202 | 237 | 241 | 291 | 316 | 318 |
| omphalodes (strain | | | | | | | | | | |
| CBS 100304) | | | | | | | | | | |
| (SEQ ID NO: 79) | | | | | | | | | | |
| Hortaea werneckii | ‘NA’ | 86 | 142 | 195 | 201 | 236 | 240 | 287 | 312 | 314 |
| (SEQ ID NO: 86) | | | | | | | | | | |
| Wallemia mellicola | ‘NA’ | 86 | 139 | 192 | 198 | 233 | 237 | 284 | 316 | 318 |
| (SEQ ID NO: 88) | | | | | | | | | | |
| Tilletia indica (SEQ | ‘NA’ | 85 | 138 | 19 | 197 | 232 | 236 | 284 | 309 | 311 |
| ID NO: 80) | | | | | | | | | | |
| Clathrospora | ‘NA’ | 89 | 145 | 199 | 205 | 240 | 244 | 291 | 316 | 318 |
| elynae (SEQ ID | | | | | | | | | | |
| NO: 81) | | | | | | | | | | |
| Neohortaea | ‘NA’ | 85 | 141 | 194 | 200 | 235 | 239 | 286 | 311 | 313 |
| acidophila (SEQ ID | | | | | | | | | | |
| NO: 98) | | | | | | | | | | |
| Cryptococcus | ‘NA’ | 82 | 135 | 188 | 194 | 229 | 233 | 282 | 307 | 309 |
| depauperatus CBS | | | | | | | | | | |
| 7841 (SEQ ID | | | | | | | | | | |
| NO: 84) | | | | | | | | | | |
| Phaeoacremonium | 72 | 89 | 144 | 200 | 206 | 241 | 245 | 292 | 317 | 319 |
| minimum UCRPA7 | | | | | | | | | | |
| (XP_007917121 2) | | | | | | | | | | |
| (SEQ ID NO: 149) | | | | | | | | | | |
| (or SEQ ID NO: 150 | | | | | | | | | | |
| with 6His Tag) | | | | | | | | | | |
| Ophiostoma piceae | 72 | 89 | 144 | 199 | 205 | 240 | 244 | 294 | 316 | 318 |
| UAMH 11346 | | | | | | | | | | |
| (EPE10171 2) | | | | | | | | | | |
| (SEQ ID NO: 153) | | | | | | | | | | |
| (or SEQ ID NO: 154 | | | | | | | | | | |
| with 6His Tag) | | | | | | | | | | |

In one or more embodiments the PAP enzyme may comprise any of those listed in the table above. In one or more embodiments any of the PAP enzymes listed in the table above may be modified at any of the positions/residues defined in the table above which correspond to those defined in the statements of the invention. In one or more embodiments therefore the invention comprises a PAP variant selected from the table above, having one or more mutations at the positions/residues identified in the table above. In one or more embodiments, there is provided a poly(A)polymerase (PAP) variant comprising an amino acid sequence having at least 70% identity to SEQ ID NO:1 and comprising one or more mutations at the positions/residues identified in Table 1. In one or more embodiments, there is provided a poly(A)polymerase (PAP) variant comprising an amino acid sequence having at least 70% identity to the sequence of a PAP enzyme identified in Table 1 and comprising one or more mutations at the positions/residues identified in Table 1.

For example, within the scope of the invention, there may be provided a poly(A) polymerase variant comprising an amino acid sequence having at least 70% identity to the PAP sequence from Trichoderma citrinoviride according to SEQ ID NO:95 and comprising mutations at positions 190, 235, and 307 of said sequence.

In one embodiment, the invention concerns a poly(A) polymerase variant comprising an amino acid sequence having at least 70% identity to the PAP sequence from Chaetomium thermophilum according to SEQ ID NO:76 and comprising mutations at positions 200, 245, and 319 of said sequence.

In one embodiment the invention concerns a poly(A) polymerase variant comprising an amino acid sequence having at least 70% identity to the PAP sequence from Phaeoacremonium minimum according to SEQ ID NO:149 and comprising mutations at positions X, Y and Z of said sequence.

the invention concerns a poly(A) polymerase variant comprising an amino acid sequence having at least 70% identity to the PAP sequence from Ophiostoma piceae according to SEQ ID NO:153 and comprising mutations at positions X, Y and Z of said sequence. In one or more embodiments the PAP variants of the invention are capable of synthesising a polynucleotide, suitably without a template. Preferentially the PAP variants of the invention are capable of synthesising a ribonucleic acid, suitably without a template. In one or more embodiments the PAP variants of the invention are capable of incorporating a nucleotide onto a polynucleotide strand. Preferentially the PAP variants of the invention are capable of incorporating a ribonucleoside triphosphate onto a ribonucleic acid strand. More preferentially the PAP variants of the invention are capable of incorporating a protected ribonucleoside triphosphate onto a ribonucleic acid strand.

Poly(A)polymerase activity can be measured by any suitable method known in the art. Originally, “poly(A)polymerase activity” reflects the ability to catalyse the template-independent addition of AMP from ATP to the 3′-end of RNA. However, this catalytic activity can also be measured by assessing the ability to incorporate any 3′-protected rNTP, such as 3′-O-azidomethyl-rCTP or 3′O-azidomethyl-rGTP, onto a ribonucleic acid strand. Any one of the tests described in the Examples may be used to determine the activity of a given PAP variant.

Typically, poly(A)polymerase activity can be measured by carrying out the method described in the Examples below, under the heading “Duplex assay of RNA synthesis”.

In one or more embodiments the PAP variants of the invention have an improved activity compared to corresponding wild-type sequence that the PAP variant is derived from.

Typically, the PAP variants of the invention display an improved activity when compared to the corresponding wild-type PAP, according to the “duplex GCC+G test” described in the Examples below as Test A.

According to the present invention, the activity of a given PAP variant is deemed to be improved if the ratio of the activity of said variant in the “duplex GCC+G test” over the activity of the corresponding wild-type PAP in said test is of at least 1.0. Preferably, the activity of a given PAP variant is deemed to be improved if the ratio of the activity of said variant in the “duplex GCC+G test” over the activity of the corresponding wild-type PAP in said test is of at least 1.1, even more preferably at least 1.2, at least 1.3, at least 1.4, at least 1.5, at least 1.6, at least 1.7, at least 1.8, at least 1.9, at least 2, at least 2.5, at least 3, at least 4, at least 5, at least 6, at least 7, at least 8, at least 9, at least 10, at least 15, at least 20.

By “corresponding wild-type PAP”, it is meant the wild-type PAP sequence from the species that the variant is derived from, i.e. the wild-type sequence prior to any of the above mutations or modifications.

Typically, the corresponding wild-type PAP can be determined by sequence alignments, as described above. The corresponding wild-type PAP sequence is the closest wild-type sequence (i.e. isolated from a given species, with no “man-made” modification) as determined by alignment.

Typically, the PAP variants having the sequences set forth in SEQ ID NO:3, 6 to 28 and 110-130 have an improved activity compared to the wild-type PAP from Thielavia having the sequence set forth in SEQ ID NO:1.

In one or more embodiments, the PAP variants of the invention show improved activity compared to the PAP variant having the sequence set forth in SEQ ID NO:2.

In one or more embodiments, the PAP variants of the invention show improved activity compared to PAP variant having the sequence as set forth in SEQ ID NO:6.

Typically, the PAP variants having the sequences set forth in SEQ ID NO:142-144 have an improved activity compared to the wild-type PAP from Chaetomium having the sequence set forth in SEQ ID NO:76.

Typically, the PAP variants having the sequences set forth in SEQ ID NO:151-152 have an improved activity compared to the wild-type PAP from Phaeoacremonium having the sequence set forth in SEQ ID NO:150.

Typically, the PAP variants having the sequences set forth in SEQ ID NO:155-156 have an improved activity compared to the wild-type PAP from Ophiostoma having the sequence set forth in SEQ ID NO:154.

In one or more embodiments the modifications made to the PAP enzymes herein provide an improvement in the stability of the enzyme, an improvement in the thermostability of the enzyme.

Thermostability of the PAP variant can be measured according to the Thermal Shift Assay described in the Examples below. In one or more embodiments the PAP variant has a Tm of at least 43° C., at least 44° C., at least 45° C., at least 46° C., at least 47° C., at least 48° C., at least 49° C., at least 50° C., at least 51° C., at least 52° C., at least 53° C., at least 54° C., at least 55° C. Suitably the modified PAP variant has a Tm of over 50° C. Preferentially the PAP variant has a Tm of between 51° C. and 56° C.

### Production of Poly(A) Polymerase Variants

The PAP variants of the invention may be obtained by various techniques well known in the art. In particular, examples of techniques for altering the DNA sequence encoding a wild-type protein, include, but are not limited to, site-directed mutagenesis, random mutagenesis and synthetic oligonucleotide construction.

The term “wild-type”, as used herein refers to the non-mutated version of a nucleic acid or protein as it appears naturally. Suitably the wild type PAP enzyme from which the variant or modified forms of the invention are derived comprises the sequence set out in SEQ ID NO:1 defined herein, equally in other embodiments, the wild type PAP enzyme may comprise one of the sequences set out in table 1 above.

In one or more embodiments, PAP variants of the invention may be produced by mutating wild type PAP-coding polynucleotides, then expressing the variant polynucleotides using conventional molecular biology techniques. For example, a desired gene or DNA fragment encoding a PAP polypeptide of desired sequence may be assembled from synthetic fragments using conventional molecular biology techniques or the like, or such gene or DNA fragment may be directly cloned from cells of a selected species using conventional protocols.

An isolated gene encoding a desired PAP variant may be inserted into an expression vector to give an expression vector which then may be used to make and express the variant PAP protein using conventional methods. Such vectors may be transformed into producer strains such as E. coli. In one or more embodiments, the isolated gene encoding a desired PAP variant is inserted into a pET28b vector using restriction sites Nco1 and Not1, which is then transformed into E. coli.

In one or more embodiments, the transformed strains are then cultured using conventional techniques to form a population of transformed cells from which the PAP variant enzyme is extracted.

In one or more embodiments, variant PAP enzymes may be purified from the centrifugate in a one-step affinity procedure. For example, Ni-NTA affinity column (GE Healthcare) may be used to bind the variant PAP polymerases. Fractions corresponding to the highest concentration of variant PAP polymerases of interest are collected and pooled in a single sample. The pooled fractions are dialyzed, concentrated and analysed in SDS-PAGE gels.

In one or more embodiments, a PAP variant enzyme may be operably linked to a linker moiety including a covalent or non-covalent bond; amino acid tag (e.g., poly-amino acid tag, poly-His tag, 6His-tag, or the like); chemical compound (e.g., polyethylene glycol); protein-protein binding pair (e.g., biotin-avidin); affinity coupling; capture probes; or any combination of these. The linker moiety can be separate from, or part of a PAP enzyme. An exemplary His-tag for use with modified PAP variants of the invention is MASSHHHHHHSSGSENLYFQTGSSG- (SEQ ID NO: 29) or GSSGENLYFQGSSGSHHHHHH (SEQ ID NO:109) or HHHHHH (SEQ ID NO:30). The tag-linker moiety does not interfere with the nucleotide binding activity, or catalytic activity of the PAP enzyme.

The above processes, or equivalent processes, result in an isolated PAP variants that may be used directly in a method of polynucleotide synthesis or which may be mixed with a variety of reagents, such as, salts, pH buffers, carrier compounds, and the like, that are necessary or useful for activity and/or preservation.

In one or more embodiments, the PAP variants of the invention may be produced as described hereinbelow in the examples.

In one or more embodiments, the PAP variants of the invention are produced with an increased yield compared to the corresponding wild-type PAP enzyme. Typically, the production yield can be increased by a factor 1.2, 1.3, 1.4, 1.5, 1.6, 1.7, 1.8, 1.9, 2.0, 2.5, 3.0, 4.0, 5.0, 10 or more, compared to the production yield of the wild-type PAP.

### Methods of Polynucleotide Synthesis

The PAP variants of the invention are useful in methods of synthesising polynucleotides, particularly in template-free methods of synthesising polynucleotides.

As defined in the statements above, the method of synthesising a polynucleotide may comprise a step of contacting a nucleic acid with a poly(A) polymerase variant of the invention and one or more nucleotides under suitable conditions to incorporate the or each nucleotide into the nucleic acid.

In one or more embodiments the method of polynucleotide synthesis is enzymatic or chemical. Preferentially enzymatic polynucleotide synthesis, preferentially template-free enzymatic synthesis. More preferentially the one or more steps of enzymatic polynucleotide synthesis may be carried out as described in WO2020/165137 for example. In one or more embodiments the steps are demonstrated in FIG. 1 which may be incorporated herein.

Preferentially therefore, the method of synthesising a polynucleotide may comprise:
(a) Providing a nucleic acid initiator having a free 3′-hydroxyl group;
(b) Contacting the nucleic acid initiator, or an elongated nucleic acid thereof, with a poly(A) polymerase variant according to the first aspect and a protected nucleotide such that the nucleic acid is elongated by incorporation of the protected nucleotide;
(c) Deprotecting the protected nucleotide of the elongated nucleic acid; and
(d) Repeating steps (b) and (c) until the polynucleotide is formed

The term ‘polynucleotide’ as used herein refers to a polymer of nucleotides. Suitably to a polymer of A, T, U, G or C nucleotides, or optionally modified or synthetic nucleotides or nucleotide analogues comprising for example a modified bond, a modified purine or pyrimidine base, or a modified sugar. The terms “polynucleotide(s)”, “nucleic acid sequence(s)”, “nucleotide sequence(s)”, “nucleic acid(s)”, “nucleic acid molecule” are used interchangeably herein and refer to nucleotides, either ribonucleotides or deoxyribonucleotides or a combination of both, in a polymeric unbranched form of any length.

In one or more embodiments the polynucleotide may be a deoxyribonucleic acid (DNA) or a ribonucleic acid (RNA) molecule or polymer, or in some cases may be a hybrid of DNA and RNA. These may be referred to as polyribonucleic acids, or polydeoxyribonucleic acids. Alternatively, the polynucleotide may be an artificial polynucleotide or nucleic acid analogue selected from PNA, LNA, GNA, TNA, and HNA. In one or more embodiments the polynucleotide may be a DNA or RNA molecule or polymer. In one or more embodiments the polynucleotide may be single stranded (ss) or double stranded (ds). In one or more embodiments the polynucleotide may be selected from ssRNA, ssDNA, dsRNA, and dsDNA. Preferentially the polynucleotide is single stranded. Preferentially the polynucleotide is an RNA molecule.

More preferentially, the polynucleotide is a single stranded RNA (ssRNA) molecule.

In one or more embodiments the polynucleotide product may be of any length. In one or more embodiments the polynucleotide product may be up to 1000 nucleotides in length, between 5 to 1000 nucleotides in length, between 5 to 900 nucleotides in length, between 5 to 800 nucleotides in length, between 5 to 700 nucleotides in length, between 5 to 600 nucleotides in length, between 10 to 500 nucleotides in length, between 10 to 400 nucleotides in length, between 10 to 300 nucleotides in length, between 10 to 200 nucleotides in length, between 10 to 100 nucleotides in length, between 10 to 50 nucleotides in length, between 10 to 40 nucleotides in length, between 10 to 30 nucleotides in length, between 10 to 20 nucleotides in length.

In one or more embodiments, the polynucleotide product may be up to 100 nucleotides in length, up to 90 nucleotides in length, up to 80 nucleotides in length, up to 70 nucleotides in length, up to 60 nucleotides in length, up to 50 nucleotides in length, or up to 40 nucleotides in length.

In one or more embodiments therefore, the polynucleotides may be referred to as ‘oligonucleotides’ when the length of the polynucleotides is lower or equal to 40 nucleotides.

In one or more embodiments a single PAP variant may be employed for coupling all nucleotides in the synthesis of a polynucleotide. Alternatively, multiple different PAP variants may be employed for coupling different nucleotides in the synthesis of a polynucleotide. In one or more embodiments therefore the methods of synthesising a polynucleotide may comprise contacting with more than one PAP variant enzyme of the invention.

In one or more embodiments the PAP variants of the invention may be used to synthesise a polynucleotide, which may be an RNA molecule, using nucleotides, preferentially using protected nucleoside triphosphates (NTPs), which may be protected ribonucleoside triphosphates (rNTPs).

Preferentially the protected nucleotide may comprise a 3′-O-protected nucleotide. Guidance in selecting 3′-O-protecting groups and corresponding deprotecting conditions for the above method may be found in the following references: U.S. Pat. Nos. 5,808,045; 8,808,988; International patent publication WO91/06678, for example.

In one or more embodiments, a deprotection agent is used to deprotect the protected nucleotide in step (c). Preferentially a deprotection agent is a chemical cleaving agent, such as, for example, dithiothreitol (DTT). Alternatively, a deprotection agent may be an enzymatic deprotection agent, such as, for example, a phosphatase, which may cleave a 3′-phosphate protecting group. It will be understood by the person skilled in the art that the selection of the deprotection agent depends on the type of 3′-nucleotide protection group used, whether one or multiple protection groups are being used, whether initiator nucleic acids are attached to living cells or organisms or to solid supports, and the like, that necessitate mild treatment. For example, a phosphine, such as tris(2-carboxyethyl)phosphine (TCEP) can be used to deprotect a 3′O-azidomethyl groups, palladium complexes can be used to deprotect a 3′O-allyl groups, or sodium nitrite can be used to deprotect a 3′O-amino group. In one or more embodiments the deprotection step involves TCEP, a palladium complex or sodium nitrite.

In one or more embodiments, it is desirable to employ two or more different protecting groups that may be removed using orthogonal deprotection conditions. The following exemplary pairs of protecting groups may be used in parallel synthesis embodiments in which two or more polynucleotide sequences are synthesised in the same reaction mixture.

It is understood that other deprotecting group pairs, or groups containing more than two, may be available for use in the invention.

TABLE 2
Protecting Group Pairs

| | |
|---|---|
| 3′-O-NH2 | 3′-O-azidomethyl |
| 3′-O-NH2 | 3′-O-allyl, 3′O-propargyl |
| 3′-O-NH2 | 3′-O-phosphate |
| 3′-O-azidomethyl | 3′-O-allyl, 3′O-propargyl |
| 3′-O-azidomethyl | 3′-O-phosphate |
| 3′-O-allyl, 3′O-propargyl | 3′-O-phosphate |

In one or more embodiments if the polynucleotide to be synthesised is RNA then the protected nucleotide is an rNTP (ribonucleoside triphosphate). In one or more embodiments therefore the elongation step may comprise between 125-500 μM protected rNTP. Preferentially protected rNTPs may be 3′-O-blocked rNTPs. Preferentially the rNTP may be a 3′-O-azidomethyl-rNTP, which may be selected from protected A, C, G and U ribonucleosides. More preferentially which may be selected from 3′-azidomethyl-O-adenosine triphosphate, 3′-azidomethyl-O-guanosine triphosphate, 3′-azidomethyl-O-cytidine triphosphate, and 3′-azidomethyl-O-uridine triphosphate. Alternatively, the 3′-blocked nucleotide triphosphate is blocked by either 3′-0-propargyl, a 3′-0-azidomethyl, 3′-0-NH₂ 3′-0-allyl group, 3′-0-methyl, 3′-0-(2-nitrobenzyl), 3′-0-allyl, 3′-0-amine, 3′-0-azidomethyl, 3′-0-tert-butoxy ethoxy, 3′-0-(2-cyanoethyl), or 3′-0-propargyl group. Any of the 3′-0-blocked rNTPs employed in the invention may be purchased from commercial vendors (e.g. Jena Bioscience, MyChemLabs, or the like) or synthesized using published techniques, e.g. U.S. patent 7057026; International patent publications WO2004/005667, WO91/06678; Canard et al, Gene (cited above); Metzker et al, Nucleic Acids Research, 22: 4259-4267 (1994); Meng et al, J. Org. Chem., 14: 3248-3252 (3006); U.S. patent publication 2005/037991; Zavgorodny et al, Tetrahedron Letters, 32(51): 7593-7596 (1991).

In one or more embodiments, the 3′-O-blocked rNTP may also contain 2′-modifications such as 2′-O-methyl-rNTPs or 2′-fluoro-rNTPs.

In one or more embodiments, exemplary reaction conditions for elongation step (b) may comprise the following: between 2-5 μM purified PAP variant enzyme; between 125-500 μM protected NTP (nucleoside triphosphate), such as a 3′-O-blocked NTP; about 10 to about 500 mM buffer (pH around 8) such as Tris-HCl; and from about 0.01 to about 5 mM of a divalent cation, suitably in a salt such as COC₂ or MnCl₂, where the elongation reaction may be carried out in a 50 pL reaction volume. Preferentially such components may be comprised within an elongation buffer.

In one or more embodiments therefore there may be provided an elongation buffer comprising a PAP variant of the invention, a protected NTP, a buffer and a divalent cation salt. Preferentially step (b) comprises contacting with an elongation buffer.

In one or more embodiments the elongation step is carried out at a temperature within the range of room temperature (21° C.) to 45° C., for between 5 to 10 minutes.

In one or more embodiments during the elongation step, the nucleic acid is elongated by incorporation of a protected nucleotide, preferentially an individual protected nucleotide. In one or more embodiments therefore, in each cycle of steps (b) to (d), an individual protected nucleotide is added to the nucleic acid. In one or more embodiments the protected nucleotide to be added is determined by the sequence of the polynucleotide to be synthesised.

In one or more embodiments, exemplary reaction conditions for deprotecting step (c) may comprise the following: between 200-600 mM deprotecting agent such as DTT; and about 200-700 mM buffer (pH around 9) such as Tris-HCl; where the deprotecting reaction may be carried out in a 50 pL reaction volume. Preferentially such components may be comprised in a deprotection buffer.

In one or more embodiments, there may be provided a deprotection buffer comprising a deprotecting agent and a buffer. Preferentially step (c) comprises contacting with a deprotection buffer.

In one or more embodiments the deprotection step is carried out at a temperature within the range of room temperature (21° C.) to 45° C., for between 5 to 15 minutes.

In one or more embodiments after the deprotecting step, the deprotected elongated nucleic acid comprises a free 3′-hydroxyl group.

In one or more embodiments, an “nucleic acid initiator” refers to a short oligonucleotide sequence with a free 3′hydroxyl group, which can be further elongated by the PAP variants of the invention. In one or more embodiments, the nucleic acid initiator is a DNA molecule. Alternatively, the nucleic acid initiator is a RNA molecule. In one or more embodiments the nucleic acid initiator comprises between 3 and 100 nucleotides, in particular between 3 and 20 nucleotides. In one or more embodiments, the nucleic acid initiator is single-stranded. Alternatively, the nucleic acid initiator is double-stranded. Preferentially, the nucleic acid initiator is a RNA molecule as the polynucleotide to be synthesised is a RNA molecule.

In one or more embodiments the nucleic acid initiator is provided on a surface. In one or more embodiments the nucleic acid initiator is immobilised on the surface. Preferentially at its 5′ end. In one or more embodiments the surface may be an inert material such as a sepharose, agarose resin, or a particle or bead, preferentially, the bead may be magnetic. In one or more embodiments a nucleic acid initiator synthesized with a 5′-primary amine may be covalently linked to magnetic beads using the manufacturer's protocol. Likewise, an initiator synthesized with a 3′-primary amine may be covalently linked to magnetic beads or agarose beads using the manufacturer's protocol. A variety of other attachment chemistries amenable for use with the invention are well-known in the art, e.g. Integrated DNA Technologies brochure, “Strategies for Attaching Oligonucleotides to Solid Supports,” v.6 (2014); Hermanson, Bioconjugate Techniques, Second Edition (Academic Press, 2008).

In one or more embodiments the nucleic acid initiator may further comprise one or more fluorescent groups or tags. In one or more embodiments the initiator may comprise a primer, preferentially a primer at its free end, which may be its 3′ end. Preferentially the primer is used by the PAP variant to begin polymerisation of the polynucleotide. A primer may be a poly rNTP sequence such as a poly rATP sequence. More preferentially, the initiator comprises a primer of (rATP)₅.

One exemplary initiator may comprise the sequence: 5′-AmMCi12/TTTTTTTTTTTTTTTTTTTT/ideoxyl/TTTTTTTTTT/iFluorT/TTTTTrArArArArA-3′ (SEQ ID NO:34), wherein ‘AmMC12’ is a 5′ Amino Modified C12 linker from IDT (idtdna.com), ‘ideoxyl’ is a modified inosine base, and ‘iFluorT’ is a modified fluorescent thymine base.

As explained below, the initiator may comprise an internal cleavage point, in this case an deoxyinosine residue.

In one or more embodiments to the initiator, or to the elongated nucleic acid in subsequent cycles of the method, are added protected nucleotides and a PAP variant of the invention which is capable of enzymatically incorporating the protected nucleotide onto the 3′ end of the nucleic acid initiator. In one or more embodiments the elongation step produces an elongated nucleic acid initiator comprising a protected 3′ hydroxyl group.

In one or more embodiments if the elongated nucleic acid after step (b) is not complete, then the protection group is removed in step (c) by deprotection to expose the free 3′-hydroxyl group. In one or more embodiments then the cycle of steps (b) to (d) is repeated to add further nucleotides to the elongated nucleic acid. In one or more embodiments therefore step (c) may comprise deprotecting the protected nucleotide of the elongated nucleic acid to form an elongated nucleic acid with a free 3′hydroxyl. In one or more embodiments the elongated nucleic acid may also be referred to as ‘an extended nucleic acid’, ‘an extension intermediate’, ‘an elongated nucleic acid fragment’, ‘an elongation fragment’.

Optionally the method may further comprise a step of cleaving the completed polynucleotide from the nucleic acid initiator, which may be after step (d). In one or more embodiments by use of an endonuclease enzyme. Preferentially by use of an EndoV enzyme. In one or more embodiments therefore the nucleic acid initiator may comprise a cleavable inosine residue. In one or more embodiments the cleavable inosine residue is penultimate to the 3′-terminal nucleotide of the initiator. Alternatively, cleavage may take place by use of a uracil DNA glycosylase enzyme. In one or more embodiments therefore the nucleic acid initiator may comprise a cleavable uracil nucleotide. Alternatively, still, a cleavable linker may be used. In one or more embodiments the cleaving step may comprise the use of a cleavage buffer, which may comprise these components. In one or more embodiments, exemplary reaction conditions for cleavage may comprise: cleaving enzyme such as 0.1-10 μM EndoV, 10 mM buffer such as Tris-HCl, (around pH—8), 10-100 mM MgCl₂, and 10-200 mM NaCl. Preferentially the cleavage step is carried out at about 37° C. for around 30 minutes.

In one or more embodiments, if the elongated nucleic acid after step (b) is complete, i.e. the polynucleotide is fully formed, then a final deprotection step (c) occurs and the polynucleotide is cleaved from the nucleic acid initiator.

In one or more embodiments the cleaving step may be carried out enzymatically, chemically, thermally, or photochemically. In one or more embodiments the cleavable nucleotide may be a nucleotide analog such as deoxyuridine (for cleavage by uracil DNA glycosylase) or deoxyinosine (for cleavage by endonuclease V). Further means of cleaving polynucleotides are disclosed in U.S. Pat. Nos. 5,739,386, 5,700,642, P5830655 for example.

In one or more embodiments, cleavage may leave a 5-hydroxyl on the polynucleotide product or may leave a moiety at the 5′ end. In such embodiments, the method may comprise a further step of removing 5′ moieties from the polynucleotide, for example by phosphatase treatment.

Optionally the method may comprise one or more wash steps, which may be between each cycle of repetition. In one or more embodiments, the wash steps are carried out with wash buffer and/or with water. In one or more embodiments the wash steps remove any unused nucleotides. In one or more embodiments there is at least a first wash after the elongation step and a second wash after the deprotection step. Preferentially the first wash step may comprise a wash to remove unincorporated nucleotides and optionally elongation buffer. Preferentially the second wash step may comprise a wash to remove protection groups (suitably the protection group that has been removed from the protected nucleotide), and optionally deprotection buffer.

In one or more embodiments, the wash buffer may comprise 0.1-1 M LiCl, 0.2 M buffer such as Tris-HCl, (around pH 7.5), and 0.01% surfactant such as Tween20.

In one or more embodiments therefore, the method of synthesising a polynucleotide may comprise:
(a) Providing a nucleic acid initiator having a free 3′hydroxyl group;
(b) Contacting the nucleic acid initiator, or an elongated nucleic acid thereof, with a poly(A) polymerase variant according to the first aspect and a protected nucleotide such that the nucleic acid is elongated by incorporation of the protected nucleotide;
(c) Washing the elongated nucleic acid to remove unincorporated protected nucleotides;
(d) Deprotecting the protected nucleotide of the elongated nucleic acid;
(e) Washing the elongated nucleic acid to remove protection groups; and
(f) Repeating steps (b), (c), (d) and (e) until the polynucleotide is formed

Optionally the above methods may further comprise a step of capping the polynucleotide. In one or more embodiments this is after the elongation and deprotection steps. In one or more embodiments a capping step may be included in which a free 3′-hydroxyl is reacted with a compound that prevents any further elongation of the capped strand. In one or more embodiments, such a compound may be a dideoxy nucleoside triphosphate. In other embodiments, elongated nucleic acids with free 3′-hydroxyls may be degraded by treating them with a 3′-exoribonuclease activity, e.g. RNase R (Epicentre) to prevent further elongation. Likewise, in one or more embodiments, strands that fail to be deprotected may be treated to either remove the strand or render it inert to further elongation.

In one or more embodiments that comprise serial synthesis of oligoribonucleotides, capping steps may be undesirable as capping may prevent the production of equal molar amounts of a plurality of oligonucleotides. Without capping, sequences will have a uniform distribution of deletion errors, but each of a plurality of oligoribonucleotides will be present in equal molar amounts. This would not be the case where non-extended fragments are capped.

In one or more embodiments once the desired polynucleotide is formed from the method of synthesis, and cleaved from the nucleic acid initiator, the polynucleotide is collected. In one or more embodiments the polynucleotide may be collected by separating it from the reaction mixture, which may be achieved by a step of centrifuging the reaction mixture to form a pellet comprising the polynucleotide.

### Polynucleotide Products

The poly(A) polymerase variants of the invention may be used to synthesise polynucleotide products. Some aspects of the invention relate to a polynucleotide produced from a method employing the poly(A) polymerase variants, and further to compositions comprising said polynucleotide products.

In one or more embodiments as described hereinabove, the polynucleotide may be a deoxyribonucleic acid (DNA) or a ribonucleic acid (RNA) molecule or polymer, or in some cases may be a hybrid of DNA and RNA. In one or more embodiments, these may be referred to as polyribonucleic acids, or polydeoxyribonucleic acids. Alternatively, the polynucleotide may be an artificial polynucleotide or nucleic acid analogue selected from PNA, LNA, GNA, TNA, and HNA. In one or more embodiments the polynucleotide may be a DNA or RNA molecule or polymer. In one or more embodiments the polynucleotide may be single stranded (ss) or double stranded (ds). In one or more embodiments the polynucleotide may be selected from ssRNA, ssDNA, dsRNA, and dsDNA. Preferentially the polynucleotide is single stranded. Preferentially the polynucleotide is an RNA molecule.

More preferentially, the polynucleotide is a single stranded RNA (ssRNA) molecule.

In one or more embodiments the polynucleotide product may be of any length. In one or more embodiments the polynucleotide product may be up to 1000 nucleotides in length, between 5 to 1000 nucleotides in length, between 5 to 900 nucleotides in length, between 5 to 800 nucleotides in length, between 5 to 700 nucleotides in length, between 5 to 600 nucleotides in length, between 10 to 500 nucleotides in length, between 10 to 400 nucleotides in length, between 10 to 300 nucleotides in length, between 10 to 200 nucleotides in length, between 10 to 100 nucleotides in length, between 10 to 50 nucleotides in length, between 10 to 40 nucleotides in length, between 10 to 30 nucleotides in length, between 10 to 20 nucleotides in length.

In one or more embodiments, the polynucleotide product may be up to 100 nucleotides in length, up to 90 nucleotides in length, up to 80 nucleotides in length, up to 70 nucleotides in length, up to 60 nucleotides in length, up to 50 nucleotides in length, or up to 40 nucleotides in length.

In one or more embodiments, the polynucleotide product may comprise a high number of C or G nucleotides. In one or more embodiments, the polynucleotide may be classed as a ‘difficult’ sequence for the poly(A) polymerase variant to synthesise. In one or more embodiments the polynucleotide may comprise at least 5%, at least 10%, at least 15%, at least 20%, at least 25%, at least 30%, at least 35%, at least 40%, at least 45%, at least 50%, at least 55%, at least 60%, at least 65%, at least 70%, at least 75% % C or G nucleotides. In one or more embodiments the polynucleotide may comprise a majority of C or G nucleotides, suitably over 50% C or G nucleotides.

In one or more embodiments the polynucleotide product has a purity of at least about 30%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or 100% across its length.

In one or more embodiments the polynucleotide product is 5×, 10×, 15×, 20×, 25×, 30×, 35×, 40×, 45×, 50× more pure than the same polynucleotide produced using a poly(A) polymerase which is not modified according to the invention.

The ‘purity’ as referred to herein is intended to mean the percentage of the total length of the polynucleotide having the correct sequence. The correct sequence being the desired sequence of the polynucleotide that is intended to be produced by the method of synthesis.

In one or more embodiments, the PAP variants of the invention are able to synthesise polynucleotides having an equivalent or increased purity compared to the same polynucleotide synthesised by the PAP variant having the sequence set forth in SEQ ID NO:6.

### Kits

The invention further provides a kit comprising a PAP variant of the invention as defined hereinabove, and one or more reagents for synthesis, amplification, and/or sequencing of a polynucleotide.

In one or more embodiments the kit may comprise one or more different PAP variants of the invention as described hereinabove.

In one or more embodiments the reagents may comprise one or more primers or nucleic acid initiators, or adapters. Suitably one or more pairs of primers. In one or more embodiments the nucleic acid initiators may be comprised on a support, for example an array, slide, gel or bead/particle as described hereinabove. In one or more embodiments the nucleic acid initiators may each comprise a free 3′hydroxyl group. In one or more embodiments the nucleic acid initiators may each further comprise a cleavable site, for example a restriction site, an inosine cleavable nucleotide, or a photocleavable linker. Suitable nucleic acid initiators are defined hereinabove.

In one or more embodiments the reagents may comprise one or more nucleotides. In one or more embodiments one or more A, U, C, T, G nucleotides. In one or more embodiments the nucleotides may be dNTPs or rNTPs, preferentially dNTPs such as dA, dU, dC, dT, or dG, or rNTPs such as rA, rU, rC, or rG. In one or more embodiments the one or more nucleotides may be protected nucleotides, suitable for template-free enzymatic synthesis of polynucleotides. In one or more embodiments the one or more nucleotides may be 3-O-protected nucleotides such as 3′-O-amino-NTPs or 3′-O-azidomethyl-NTPs. Suitably 3′-O-amino-rNTPs or 3′-O-azidomethyl-rNTPs as listed hereinabove. In one or more embodiments the reagents may comprise a plurality of nucleotides or protected nucleotides, in one or more embodiments a plurality of each type of nucleotide selected from A, U, C, T, G, in one or more embodiments which may be dNTPs or rNTPs, or both. In one or more embodiments, the kit may comprise a mixture of nucleotides, preferentially a mixture in the required ratios of each nucleotide for the polynucleotide to be synthesised. In one or more embodiments, the kit comprises a plurality of protected rNTPs of one or more of adenosine, guanosine, uridine and cytidine. In one or more embodiments, the kit comprises a plurality of 3′-O-azidomethyl-rNTPs of one or more of adenosine, guanosine, uridine and cytidine.

In one or more embodiments, the reagents may comprise 2′-modified nucleotides such as 2′-O-methyl-rNTPs or 2′-fluoro-rNTPs.

In one or more embodiments the reagents may comprise one or more further enzymes. In one or more embodiments a polymerase enzyme, suitably an RNA or DNA polymerase. Preferentially, a Taq polymerase. Alternatively, a terminal deoxynucleotidyl transferase (TdT) enzyme. In one or more embodiments the reagents may further comprise an endonuclease enzyme capable of cleaving polynucleotides. Preferentially a DNA endonuclease. More preferentially EndoV. In one or more embodiments, the reagents may comprise a uracil DNA glycosylase enzyme. In one or more embodiments the endonuclease and/or the glycosylase is for cleaving the polynucleotide from the solid support, if present.

In one or more embodiments the reagents may further comprise one or more buffers, salts, stabilisers, chelating agents, dyes and the like. In one or more embodiments the reagents may further comprise deprotection buffer for deprotecting the protected nucleotides, if present. In one or more embodiments the reagents may comprise wash buffer for washing away unreacted nucleotides or protection groups. In one or more embodiments the reagents may comprise purification reagents, such as a column, beads, desalting buffer, eluting buffer and the like.

In one or more embodiments, there is provided a kit comprising a PAP variant of the invention as defined hereinabove, and one or more reagents for synthesis of a polynucleotide, preferentially for enzymatic synthesis of a polynucleotide, more preferentially for template-free enzymatic synthesis of a polynucleotide. In one or more embodiments the kit comprises a nucleic acid initiator on a solid support, one or more protected ribonucleotides, a PAP variant of the invention, a deprotection agent, and an endonuclease enzyme. In one or more embodiments the kit comprises a nucleic acid initiator on a solid support, an elongation buffer comprising one or more protected ribonucleotides, and a PAP variant of the invention, a deprotection buffer comprising a deprotection agent, and a cleavage buffer comprising an endonuclease enzyme. Optionally the kits further comprise wash buffer. In one or more embodiments, the kit comprises a nucleic acid initiator on a solid support, an elongation buffer comprising one or more protected ribonucleotides and a PAP variant of the invention, a deprotection buffer comprising a deprotection agent, a cleavage buffer comprising an endonuclease enzyme, and a wash buffer.

### Nucleic Acids, Vectors and Host Cells

The invention further provides nucleic acids encoding poly(A) polymerase variants of the invention as defined hereinabove, and corresponding vectors comprising the nucleic acids and host cells comprising either the proteins or the nucleic acids.

In one or more embodiments therefore, there is provided a nucleic acid molecule comprising a nucleotide sequence which encodes a PAP variant of the invention. Preferentially, the nucleotide sequence encodes a PAP variant comprising an amino acid sequence according to any of SEQ ID NO:3, 6-28, 110-130, 142-145, 151-152, 155-156, 158 and 159 or a sequence having at least 70%, 75%, 80%, 85%, 90%, 95%, 96%, 97%, 98%, 99% identity thereto. Preferentially, the nucleotide sequence encodes a PAP variant consisting of an amino acid sequence according to any of SEQ ID NO:3, 6-28, 110-130, 142-145, 151-152, 155-156, 158 and 159.

In one or more embodiments, the nucleotide sequence may be codon optimised. Preferentially the nucleotide sequence is codon optimised for optimal expression in the desired host cell, wherein the host cell is used to produce the PAP variant proteins as described hereinbelow. Any suitable means of codon optimisation in the art may be used.

Nucleic acid molecules having nucleotide sequences encoding any desired sequence, such as that of a PAP variant of the invention may be ordered or obtained from any reagents company as is known in the art for example Integrated DNA Technologies.

In one or more embodiments, the invention also encompasses nucleic acids which hybridize, under stringent conditions, to a nucleic acid encoding a poly(A) polymerase variant of the invention as defined above. Such stringent conditions include incubations of hybridization filters at about 42° C. for about 2.5 hours in 2×SSC/0.1% SDS, followed by washing of the filters four times of 15 minutes in 1×SSC/0.1% SDS at 65° C. Protocols used are described in such reference as Sambrook et al. (Molecular Cloning: a Laboratory Manual, Cold Spring Harbor Press, Cold Spring Harbor N.Y. (1988)) and Ausubel (Current Protocols in Molecular Biology (1989)).

Optionally the nucleic acid molecule may be isolated.

An “isolated” nucleic acid molecule is substantially separated away from other nucleic acid sequences with which the nucleic acid is normally associated, such as, from the chromosomal or extrachromosomal DNA of a cell in which the nucleic acid naturally occurs. A nucleic acid molecule may be an isolated nucleic acid molecule when it comprises a transgene or part of a transgene present in the genome of another organism. The term also embraces nucleic acids that are biochemically purified so as to substantially remove contaminating nucleic acids and other cellular components. Isolated nucleic acids are substantially free of sequences (preferably protein encoding sequences) that naturally flank the nucleic acid (i.e., sequences located at the 5′ and 3′ ends of the nucleic acid) in the genomic DNA of the organism from which the nucleic acid is derived. For example, the isolated nucleic acid molecule can contain less than about 5 kb, 4 kb, 3 kb, 2 kb, 1 kb, 0.5 kb, or 0.1 kb of nucleotide sequences that naturally flank the nucleic acid molecule in genomic DNA of the cell from which the nucleic acid is derived. The isolated nucleic acid molecule may be flanked by its native genomic sequences that control its expression in the cell, for example, the native promoter, or native 3′ untranslated region.

In one or more embodiments the nucleic acid molecule may be comprised upon a vector, preferentially an expression vector. The term “vector” refers to DNA molecule used as a vehicle to transfer recombinant genetic material into a host cell. The major types of vectors are plasmids, bacteriophages, viruses, cosmids, and artificial chromosomes. The vector itself is generally a DNA sequence that consists of an insert (a heterologous nucleic acid sequence, transgene) and a larger sequence that serves as the “backbone” of the vector. The purpose of a vector which transfers genetic information to the host is typically to isolate, multiply, or express the insert in the target cell.

In one or more embodiments the vector can be either RNA or DNA, either prokaryotic or eukaryotic, and typically is a virus or a plasmid. In one or more embodiments the vector is a plasmid. Suitable vectors include any vector for bacterial expression, such as pET vectors, preferentially pET28.

In one or more embodiments the expression vector may further comprise one or more regulatory elements to aid expression of the nucleic acid molecule. The term “regulatory element” or “regulatory sequence” as used herein refers to a nucleic acid that is capable of regulating the transcription and/or translation of an operably linked nucleic acid molecule. Regulatory elements include, but are not limited to, promoters, enhancers, introns, 5′ UTRs, and 3′ UTRs. For example, the expression vector may contain a promoter regulatory region (e.g., a regulatory region controlling inducible or constitutive, environmentally- or developmentally-regulated, or cell- or tissue-specific expression), a transcription initiation start site, a ribosome binding site, an RNA processing signal, a transcription termination site, and/or a polyadenylation signal. Such a portion of an expression vector may be referred to as an expression cassette.

“Expression cassette” as used herein means a nucleic acid sequence capable of directing expression of a particular nucleic acid sequence in an appropriate host cell, comprising a promoter operably linked to the nucleic acid sequence, in this case a nucleic acid molecule comprising a sequence encoding a PAP variant, which is operably linked to termination signal sequences. It also typically comprises sequences required for proper translation of the nucleic acid sequence. The expression cassette comprising the nucleic acid sequence may be chimeric, meaning that at least one of its components is heterologous with respect to at least one of its other components. The expression cassette may also be one that is naturally occurring but has been obtained in a recombinant form useful for heterologous expression. Typically, however, the expression cassette is heterologous with respect to the host, i.e., the particular nucleic acid sequence of the expression cassette does not occur naturally in the host cell. The expression of the nucleic acid molecule in the expression cassette may be under the control of, for example, a constitutive promoter or of an inducible promoter that initiates transcription only when the host cell is exposed to some particular external stimulus.

Expression cassettes may include in the 5′-3′ direction of transcription, a transcriptional and translational initiation region (e.g., a promoter), a nucleic acid molecule comprising a sequence encoding a PAP variant of the invention, and a transcriptional and translational termination region (e.g., termination region).

In one or more embodiments, the expression vector or expression cassette may comprise in the 5′-3′ direction of transcription, a 5′UTR, a promoter, a nucleic acid molecule comprising a sequence encoding a PAP variant of the invention, and a 3′UTR. In one or more embodiments the 5′UTR, the promoter and the nucleic acid are operably linked.

Any promoter can be used in the production of the expression cassettes and vectors including such expression cassettes as described herein. The promoter may be native or analogous, or foreign or heterologous, to the host and/or to the nucleic acid sequence. Additionally, the promoter may be a natural sequence or alternatively a synthetic sequence. Where the promoter is “foreign” or “heterologous” to the host, it is intended that the promoter is not found in the native host into which the promoter is introduced. Where the promoter is “foreign” or “heterologous” to the nucleic acid molecule, it is intended that the promoter is not the native or naturally occurring promoter for the operably linked nucleic acid molecule. Any promoter can be used in the preparation of expression cassettes to control the expression of the nucleic acid molecule. In one or more embodiments, the promoter is the native promoter of the PAP variant, suitably of the wild type PAP enzyme from which the variant enzyme is derived.

The expression cassettes may also comprise transcription termination regions. Where transcription terminations regions are used, any termination region may be used in the preparation of the expression cassettes. For example, the termination region may be native to the transcriptional initiation region, may be native to the operably linked nucleic acid molecule, may be native to the host, or may be derived from another source (i.e., foreign or heterologous to the promoter, the nucleic acid molecule of the invention, the host, or any combination thereof).

In addition, other sequence modifications can be made to the nucleic acid molecules of the invention. For example, additional sequence modifications that are known to enhance gene expression in a cellular host. These include elimination of sequences encoding spurious polyadenylation signals, exon/intron splice site signals, transposon-like repeats, and other such well-characterized sequences that may be deleterious to gene expression. The G-C content of the sequence may also be adjusted to levels average for a target cellular host, as calculated by reference to known genes expressed in the host cell. In addition, the sequence can be modified to avoid predicted hairpin secondary mRNA structures.

In preparing the expression cassettes and expression vectors described herein, the various nucleic acid molecules may be manipulated, so as to provide for the nucleic acid molecules in the proper orientation and, as appropriate, in the proper reading frame. Toward this end, adapters or linkers may be employed to join the nucleic acid molecules or other manipulations may be involved to provide for convenient restriction sites, removal of superfluous nucleic acid molecules, removal of restriction sites, or the like. For this purpose, in vitro mutagenesis, primer repair, restriction, annealing, resubstitutions, e.g., transitions and transversions, may be involved.

Expression vectors may include additional features. For example, they may include additional features such as selectable markers, e.g. Phosphomannose Isomerase (PMI), and antibiotic resistance genes that can be used to aid recovery of stably transformed hosts.

By “operably linked” or “operably associated” as used herein, it is meant that the indicated elements are functionally related to each other, and are also generally physically related. Thus, the term “operably linked” or “operably associated” as used herein, refers to nucleotide sequences on a single nucleic acid molecule that are functionally associated. Thus, a first nucleotide sequence or nucleic acid molecule that is operably linked to a second nucleotide sequence or nucleic acid molecule, means a situation when the first nucleotide sequence or nucleic acid molecule is placed in a functional relationship with the second nucleotide sequence or nucleic acid molecule. For instance, a promoter is operably associated with a nucleotide sequence or nucleic acid molecule if the promoter effects the transcription or expression of said nucleotide sequence or nucleic acid molecule. Those skilled in the art will appreciate that the control sequences (e.g., promoter) need not be contiguous with the nucleotide sequence or nucleic acid molecule to which it is operably associated, as long as the control sequences function to direct the expression thereof. Thus, for example, intervening untranslated, yet transcribed, sequences can be present between a promoter and a nucleotide sequence or nucleic acid molecule, and the promoter can still be considered “operably linked” to or “operatively associated” with the nucleotide sequence or nucleic acid molecule.

Optionally, the PAP variants of the invention may be produced by industrial fermentation of host cells engineered to express and produce the PAP variant enzymes.

The term “expression”, as used herein, refers to any step involved in the production of a polypeptide including, but not being limited to, transcription, post-transcriptional modification, translation, post-translational modification, and secretion.

In one or more embodiments the host cell of the invention may be any cell from any organism. In one or more embodiments the host cell may be a prokaryotic or eukaryotic cell. In one or more embodiments the host cell may be a bacterial, fungal, plant, insect or animal cell. Preferentially the host cell is a bacterial cell. In one or more embodiments the bacterial cell may be any bacterial cell typically used for industrial fermentation process, for the production of proteins on an industrial scale. Preferentially the host cell is an E. coli cell. More preferentially an E. coli cell selected from BL21 DE3.

In one or more embodiments the host cell may be transformed, transfected or transduced in a transient or stable manner with a nucleic acid of the invention, encoding a PAP variant. In one or more embodiments the host cell may be transformed, transfected or transduced in a transient or stable manner with a vector or expression cassette which comprises a nucleic acid of the invention encoding a PAP variant. Suitable means of transforming, transfecting or transducing a host cell are well known in the art.

The nucleic acid, expression cassette or expression vector according to the invention may be introduced into the host cell by any method known by the skilled person, such as electroporation, conjugation, transduction, competent cell transformation, protoplast transformation, protoplast fusion, biolistic “gene gun” transformation, PEG-mediated transformation, lipid-assisted transformation or transfection, chemically mediated transfection, lithium acetate-mediated transformation, liposome-mediated transformation.

Optionally, more than one copy of a nucleic acid, cassette or vector of the present invention may be inserted into a host cell to increase production of the PAP variant.

In one or more embodiments the host cell may be cultured, in one or more embodiments in a fermentation medium, preferentially in an industrial fermentation process, under suitable conditions to express a nucleic acid encoding a PAP variant of the invention. Suitable such culture conditions and suitable mediums for the production of proteins are well known in the art.

In one or more embodiments the host cells may secrete the PAP variant enzymes into the fermentation medium. Alternatively, the host cells may be lysed to release the PAP variant enzymes into the fermentation medium. In one or more embodiments the PAP variant enzymes are recovered from the fermentation medium ready for use in the methods described herein.

### DESCRIPTION OF THE FIGURES

The invention will now be described with reference to the following figures in which:

FIG. 1 shows: a flow diagram of a typical template free polynucleotide synthesis process in which a predetermined ribonucleotide monomer for RNA synthesis (or 2′-deoxyribonucleotide monomer for DNA synthesis) is added in each cycle. Initiator polynucleotides (100) are provided, for example, attached to solid support (102), which have free 3′-hydroxyl groups (103). For synthesizing RNA, typically initiators are polyribonucleotides, and for synthesizing DNA, typically initiators are polydeoxyribonucleotides. To the initiator polynucleotides (100) (or elongated initiator polynucleotides in subsequent cycles) are added a 3′-0-reversibly protected-rNTP (or 3′-0-reversibly protected-dNTP in case of DNA synthesis) and a PAP enzyme under conditions (104) effective for the enzymatic incorporation of the 3′-0-protected-rNTP (or 3′-0-protected dNTP) onto the 3′ end of the initiator polynucleotides (100) (or elongated initiator polynucleotides). This reaction produces elongated initiator polynucleotides whose 3′-hydroxyls are protected (106). If further nucleotides need to be added to the elongated polynucleotides, they are deprotected by contacting with a deprotection agent (108) to expose free 3′-hydroxyl groups (103) and the cycle repeats. If the polynucleotide is complete, then the elongated polynucleotides are deprotected by contacting with a deprotection agent and cleaved from the initiator nucleic acids (100), released from the support (100) and collected (110).

FIG. 2 shows: the synthesis of 10mer/20mer polynucleotide sequences 14, 15, 18, 3, 17, and 23 (defined below) by poly(A) polymerase mutants having the sequences set forth in SEQ ID NO:6, 16, 17 and 18.

FIG. 3 shows: the synthesis of 10mer/20mer polynucleotide sequences 20, 2, 24, 6, 5, and 9 (defined below) by poly(A) polymerase mutants having the sequences set forth in SEQ ID NO:6, 16, 17 and 18.

FIG. 4 shows: the synthesis of 10mer/20mer polynucleotide sequences 1, 19, 8, 21, 22, and 7 (defined below) by poly(A) polymerase mutants having the sequences set forth in SEQ ID NO:6, 16, 17 and 18.

FIG. 5 shows: the synthesis of 10mer/20mer polynucleotide sequences 4, 16, 13, 12, 11, and 10 (defined below) by poly(A) polymerase mutants having the sequences set forth in SEQ ID NO:6, 16, 17 and 18.

FIG. 6 shows: the synthesis of longer 20mer and 40mer polyUTP polynucleotide sequences (defined below) by poly(A) polymerase mutant having SEQ ID NO:2 and their purity

FIG. 7 shows: The reaction rates of each of the poly(A) polymerase mutants determined by duplex assay, (A) shows the reaction rates of all mutant enzymes described herein, And (B) shows the reaction rates of a subset of mutant enzymes described herein FIG. 8 shows: the alignment between SEQ ID NO: 28 and SEQ ID NO:1. The % identity between these sequences is 78%.

FIG. 9 shows: the alignment between SEQ ID NO: 127 and SEQ ID NO:1. The % identity between these sequences is 73.1%.

FIG. 10 shows: the alignment between SEQ ID NO: 76 and SEQ ID NO:1. The % identity between these sequences is 72.7%.

FIG. 11 shows: the reaction rate of mutant enzymes having SEQ ID NO: 6, 15-20, 22, 27-28 and 123-130 for the incorporation of rGTP, O-methyl-rGTP and fluoro-rGTP in the “duplex GCC+G test”

FIG. 12 shows: the reaction rate of mutant enzymes having SEQ ID NO: 6, 15-20, 22, 27-28 and 123-130 for the incorporation of rGTP, O-methyl-rGTP and fluoro-rGTP in the “duplex CGA+C test”.

FIG. 13 shows: the alignment between SEQ ID NO: 148 and SEQ ID NO:152. The % identity between these sequences is 70.2%.

FIG. 14 shows: the alignment between SEQ ID NO: 148 and SEQ ID NO:156. The % identity between these sequences is 67%.

### EXAMPLES

The invention will now be described with reference to the following non-limiting examples:

### Example 1

### Materials & Methods

### Production of Poly A Polymerase Variant Proteins

Variants of the invention may be produced by mutating known reference or wild type PAP-coding, then expressing it using conventional molecular biology techniques. For example, a desired gene or DNA fragment encoding a polypeptide of desired sequence may be assembled from synthetic fragments using conventional molecular biology techniques, e.g. using protocols described by Stemmer et al, Gene, 164: 49-53 (1995); Kodumal et al, Proc. Natl. Acad. Sci., 101: 15573-15578 (2004); or the like, or such gene or DNA fragment may be directly cloned from cells of a selected species using conventional protocols.

An isolated gene encoding a desired PAP variant may be inserted into an expression vector, such as pET-28b (Novagen), to give an expression vector which then may be used to make and express variant PAP proteins using conventional protocols. Vectors with the correct sequence may be transformed in E. coli producer strains.

Transformed strains are cultured using conventional techniques to pellets from which PAP protein is extracted. For example, previously prepared pellets are thawed in 30 to 37° C. water bath. Once fully thawed, pellets are resuspended in lysis buffer composed of 50 mM tris-HCL (Sigma) pH 7.5, 150 mM NaCl (Sigma), 0.5 mM mercaptoethanol (Sigma), 5% glycerol (Sigma), 20 mM imidazole (Sigma) and 1 tab for 100 mL of protease cocktail inhibitor (Thermofisher). Careful resuspension is carried out in order to avoid premature lysis and remaining of aggregates. Resuspended cells are lysed through several cycles of French press, until full color homogeneity is obtained. Usual pressure used is 14,000 psi. Lysate is then centrifuged for 1 h to 1 h30 at 10,000 rpm. Centrifugate is pass through a 0.2 μm filter to remove any debris before 30 column purification.

PAP protein may be purified from the centrifugate in a one-step affinity procedure. For example, Ni-NTA affinity column (GE Healthcare) may be used to bind the PAP polymerases. Initially the column is washed and equilibrated with 15 column volumes of 50 mM tris-HCL (Sigma) pH 7.5, 150 mM NaCl (Sigma) and 20 mM imidazole (Sigma). PAP polymerases are bound to the column after equilibration; then, a washing buffer, for example, composed of 50 mM tris-HCL (Sigma) pH 7.5, 500 mM NaCl (Sigma) and 20 mM imidazole (Sigma), may be applied to the column for 15 column volumes. After such washing, the PAP polymerases are eluted with 50 mM tris-HCL (Sigma) pH 7.5, 500 mM NaCl (Sigma) and 0.5M imidazole (Sigma). Fractions corresponding to the highest concentration of PAP polymerases of interest are collected and pooled in a single sample. The pooled fractions are dialyzed against the dialysis buffer (20 mM Tris-HCl, pH 6.8, 200 mM Na Cl, 50 mM MgOAc, 100 mM [NH4]2SO4). The dialysate is subsequently concentrated with the help of concentration filters (Amicon Ultra-30, Merk Millipore). Concentrated enzyme is distributed in small aliquots, 50% glycerol final is added, and those aliquots are then frozen at −20° C. and stored for long term. 5 μL of various fraction of the purified enzymes are analyzed in SDSPAGE gels.

In some embodiments, a PAP variant may be operably linked to a linker moiety including a covalent or non-covalent bond; amino acid tag (e.g., poly-amino acid tag, poly-His tag, 6His-tag, or the like); chemical compound (e.g., polyethylene glycol); protein-protein binding pair (e.g., biotin-avidin); affinity coupling; capture probes; or any combination of these. The linker moiety can be separate from or part of a PAP variant. An exemplary His-tag for use with PAP variants of the invention is GSSGENLYFQGSSGSHHHHHH (SEQ ID NO:109). The tag-linker moiety does not interfere with the nucleotide binding activity, or catalytic activity of the PAP variant.

The above processes, or equivalent processes, result in an isolated PAP variant that may be mixed with a variety of reagents, such as, salts, pH buffers, carrier compounds, and the like, that are necessary or useful for activity and/or preservation.

### General Conditions of Automated RNA Synthesis

All RNA synthesis reactions were carried out in 96 well format on a liquid handler (Hamilton Vantage) which was equipped with a shaker-incubator-vacuum unit, various 96 well plates containing buffers (elongation buffer, deprotection buffer, wash buffer, water) and pipette tips for liquid transfer.

The RNA synthesis reaction was carried out on an oligo-functionalized solid support. Initiator oligonucleotides were conjugated to a Sepharose resin via a 5′amino group. The oligonucleotide contained: i) an internal deoxyinosine base for enzymatic cleavage by endonuclease EndoV, ii) an internal fluorescence tag (iFluorT) and iii) five rATP bases at the 3′-end for priming of the enzymatic RNA synthesis reaction:

(SEQ ID NO: 34)
5′AmMC12/TTTTTTTTTTTTTTTTTTTT/ideoxyl/TTTTTTTTTT/
iFluorT/TTTTTrArArArArA

A quantity of 50 pmol or 500 pmol (quantity based on oligonucleotide primer) of functionalized resin comprising initiator nucleic acids was transferred into a 96 well filter plate (Agilent, polyethylene filter material, 25 μm pore size, 300 μL volume). The plate was installed on the shaker-incubator-vacuum unit of the Hamilton liquid handler. Vacuum was applied to remove residual liquid from the resin.

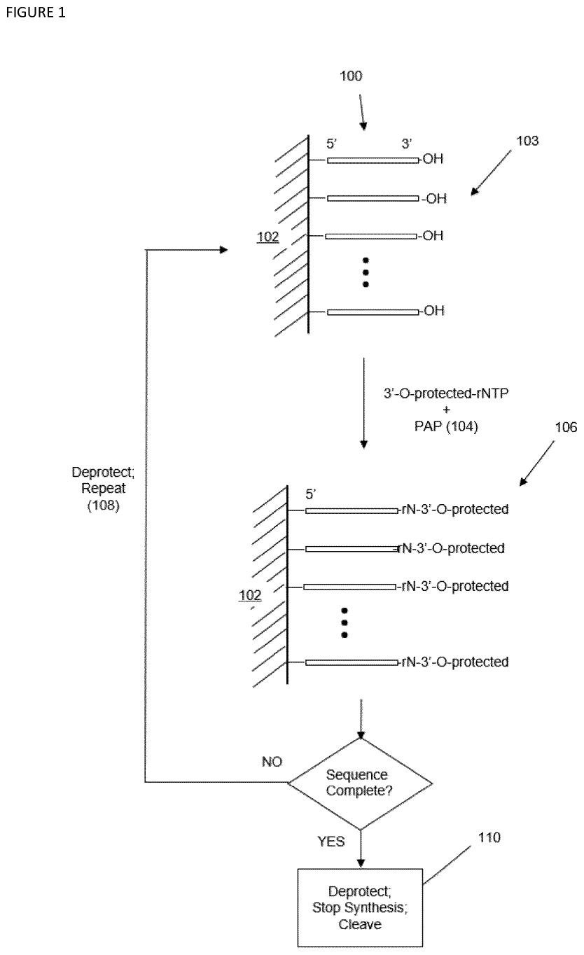

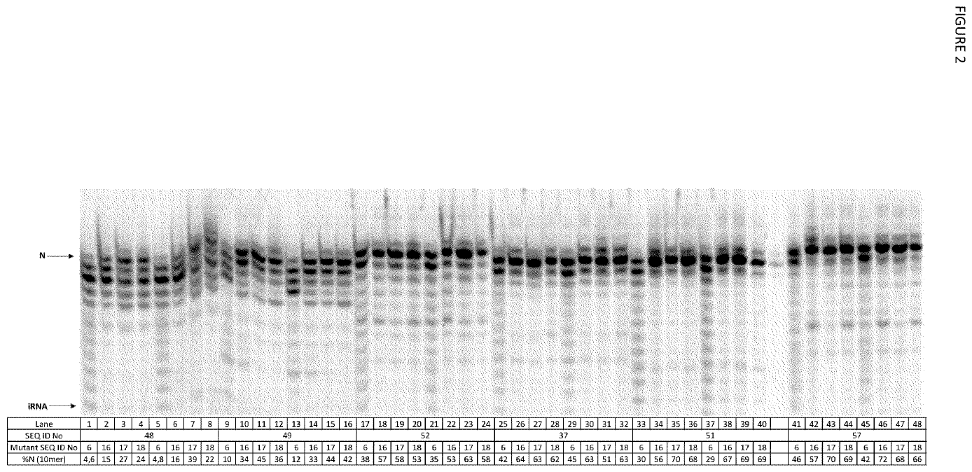

The RNA synthesis process consisted of 6 steps: 1) nucleotide elongation on, 2) resin wash with wash buffer, 3) resin wash with water, 4) nucleotide deprotection, 5) resin wash with wash buffer, 6) resin wash with water (see FIG. 1). The process was repeated until the final oligonucleotide length was obtained (eg 3, 10, 20, 40mer).

For a 50 pmol scale reaction (based on RNA primer), 50 μL elongation buffer (2 μM PAP mutant, 125 μM of one of four nucleotides, A, U, G or C, containing a 3′Oazidomehtyl protecting group, 10 mM Tris-HCl, 1 mM MnCl2) was added to the resin. The reaction was incubated at 37° C., 1000 rpm for 7 min. Vacuum of 600 mbar was applied for 15 sec to remove elongation buffer. 150 μL wash buffer (0.5 M LiCl, 0.2 M Tris-HCl, pH 7.5, 0.01% Tween20) was added to the resin and the mixture incubated for 30 sec at 1000 rpm. Vacuum was applied to remove all wash buffer. 150 μL water was added to the resin and the mixture incubated for 30 sec at 1000 rpm. Vacuum was applied to remove all water. Next, 50 μL deprotection buffer (200 mM OTT, 200 mM Tris-HCl pH 8 was added to the resin. The reaction was incubated at 37° C., 1000 rpm for 10 min. Vacuum was applied to remove the deprotection buffer. Finally, the resin was washed in wash buffer followed by water as described above.

The RNA polynucleotide product was cleaved from the resin using the following conditions: a microtiter plate was attached underneath the filter plate. 50 μL cleavage buffer (0.2μM EndoV, 10 mM Tris-HCl pH 8, 50 mM MgCl₂, 100 mM NaCl) was added to the resin and the reaction incubated for 30 min at 37° C. The plate was centrifuged 5 min, 4000 rpm to collect the final RNA product in the microtiter plate.

Two scales of reaction were applied: 50 pmol or 500 pmol the only difference being the amount of resin and the composition of the elongation buffer used (see table 7 below).

### 1. RNA Synthesis Setups

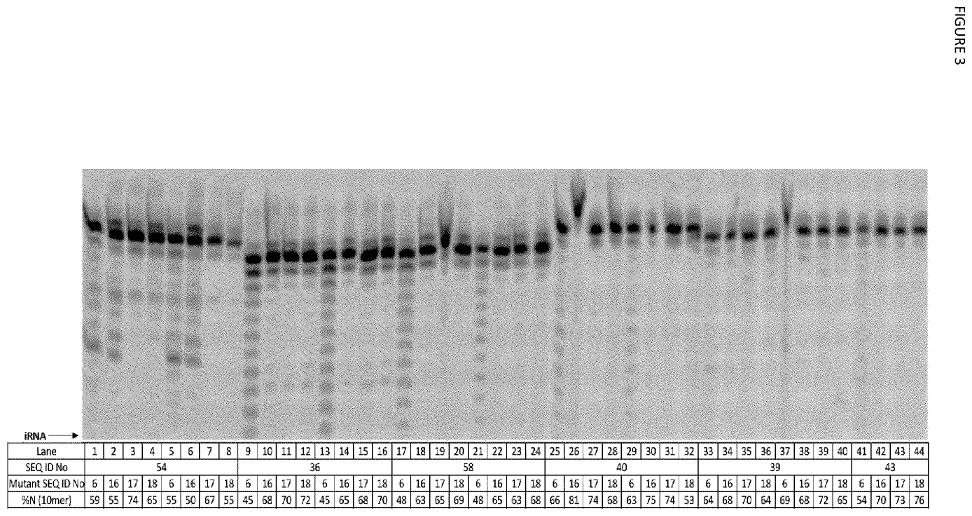

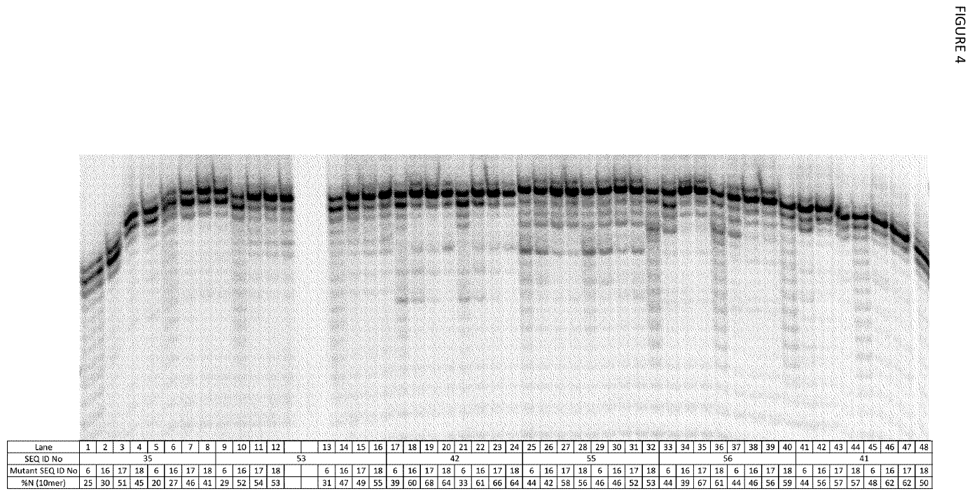

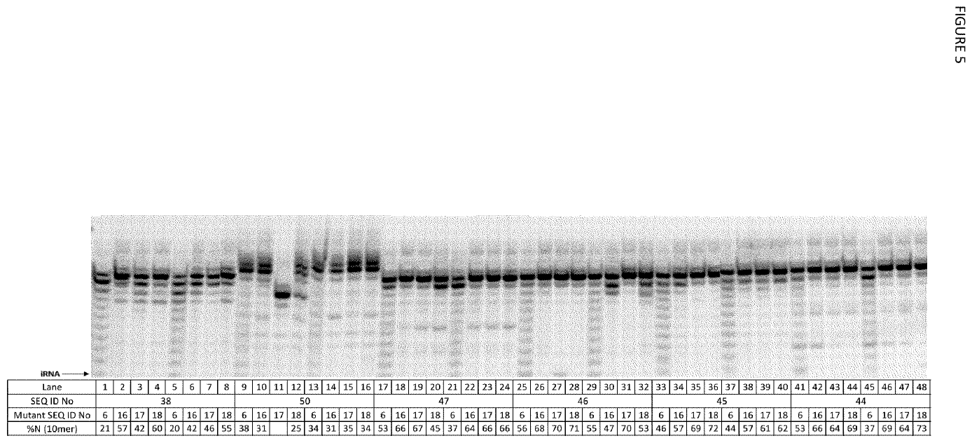

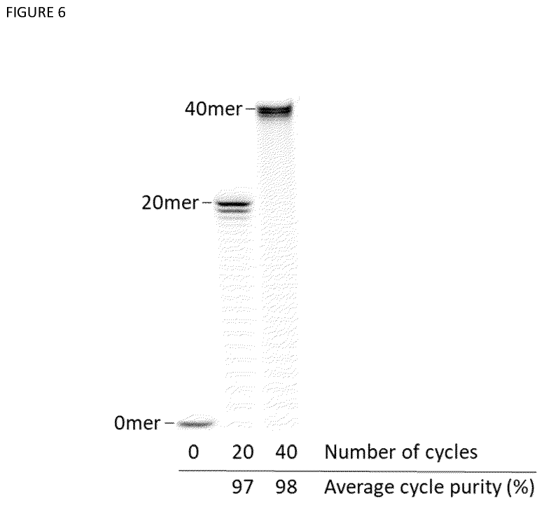

TABLE 7
Various synthesis setups were used in this work.

| | | | | | | 20mer and |
|---|---|---|---|---|---|---|
| Synthesis | 3x | 24x | | | | 40mer |
| type | 10mers | 10mers | 6x 4mers | 9x 20mers | 3x 10mers | polyU |
| Results | Table 8 | FIGS. 2-5 | Table 8 | Table 8 | Table 8 | FIG. 6 |
| shown in | | and Table 8 | | | | |
| Mutants | 2, 3, 4, 5, | 6, 7, 8, 9, | 18, 19, 22 | 6, 20, 21, | 22, 23 24, | 2 |
| (SEQ ID NO) | 6 | 10, 14, 15, | | 22 | 26, 25 27, | |
| | | 16, 17, 18 | | | 28 | |
| Scale (pmol) | 50 | 50 | 500 | 500 | 500 | 50 |
| Elongation | 2 μM | 2 μM PAP, | 2 μM PAP, | 2 μM PAP, | 2 μM PAP, | 2 μM PAP, |
| buffer | PAP | 125 UM | 125 μM | 125 μM | 125 μM | 125 μM |
| | 125 μM | 3′AM-rNTP, | 3′AM-rNTP, | 3′AM-rNTP, | 3′AM-rNTP, | 3′AM-rNTP, |
| | 3′AM- | 10 mM | 10 mM | 10 mM | 10 mM | 10 mM |
| | rNTP, 10 | TrisHCl, | TrisHCl, | TrisHCl, | TrisHCl, | TrisHCl, |
| | mM | pH 7.5, | pH 7.5, | pH 7.5, | pH 7.5, | pH 7.5, |
| | TrisHCl, | 1 mM | 1 mM | 1 mM | 1 mM | 1 mM |
| | pH 7.5, | MnCl2 | MnCl2 | MnCl2 | MnCl2 | MnCl2 |
| | 1 mM | | | | | |
| | MnCl2 | | | | | |
| Analysis | Agarose | Agarose gel | Agarose gel | Capillary | Agarose gel | Agarose gel |
| | gel | | | Electro- | | |
| | | | | phoresis | | |
| Sequences | 5) | 1) | 1) GACC | 1) | 1) | 1) |
| to be | AUCCGU | GGCCCAG | (SEQ ID | GAUUGCG | GGCCCAG | UUUUUUU |
| Synthesised | AGCC | AUG (SEQ | NO: 59) | GGCGAUG | AUG (SEQ | UUUUUUU |
| | (SEQ ID | ID NO: 35) | 2) CCCC | GGUGAG | ID NO: 35) | UUUUUU |
| | NO: 39) | 2) | (SEQ ID | (SEQ ID | 15) | (SEQ ID |
| | 7) | UUCUUCU | NO: 60) | NO: 65) | AGGGUCC | NO: 74) |
| | CAGCGC | CCU (SEQ | 3) AAAA | 2) | CCA (SEQ | 2) |
| | GCAC | ID NO: 36) | (SEQ ID | AAGCAGA | ID NO: 49) | UUUUUUU |
| | (SEQ ID | 3) | NO: 61) | UCAAAUG | 24) | UUUUUUU |
| | NO: 41) | GGCCAAA | 4) GUCG | UGUAGA | CCACCAA | UUUUUUU |
| | 13) | ACC (SEQ | (SEQ ID | (SEQ ID | ACU (SEQ | UUUUUUU |
| | UGUAAC | ID NO: 37) | NO: 62) | NO: 66) | ID NO: 58) | UUUUUUU |
| | ACCA | 4) | 5) UUCC | 3) | | UUUUU |
| | (SEQ ID | GCCUGUU | (SEQ ID | AUCAAUC | | (SEQ ID |
| | NO: 47) | CAG (SEQ | NO: 63) | CUAGUAC | | NO: 75) |
| | | ID NO: 38) | 6) CGGG | AUCCGC | | |
| | | 5) | (SEQ ID | (SEQ ID | | |
| | | AUCCGUA | NO: 64) | NO: 67) | | |
| | | GCC (SEQ | | 4) | | |
| | | ID NO: 39) | | GCAUGAA | | |
| | | 6) | | CCUUUCG | | |
| | | GGGGCUG | | ACAUCU | | |
| | | UAC (SEQ | | (SEQ ID | | |
| | | ID NO: 40) | | NO: 68) | | |
| | | 7) | | 5) | | |
| | | CAGCGCG | | ACACUGA | | |
| | | CAC (SEQ | | CAGUGCU | | |
| | | ID NO: 41) | | CAAUAA | | |
| | | 8) | | (SEQ ID | | |
| | | AGGUCCU | | NO: 69) | | |
| | | CAC (SEQ | | 6) | | |
| | | ID NO: 42) | | AAGAAUC | | |
| | | 9) | | AGUAAAG | | |
| | | CCCAGGC | | UAUGGG | | |
| | | GCA (SEQ | | (SEQ ID | | |
| | | ID NO: 43) | | NO: 70) | | |
| | | 10) | | 7) | | |
| | | ACGUGCU | | CCGACUG | | |
| | | GCC (SEQ | | AUAACCG | | |
| | | ID NO44) | | CGACGG | | |
| | | 11) | | (SEQ ID | | |
| | | CCGCAUC | | NO: 71) | | |
| | | AGC (SEQ | | 8) | | |
| | | ID NO: 45) | | AGUUACU | | |
| | | 12) | | AUAUCAU | | |
| | | CGCCGCA | | CAGCGG | | |
| | | UCA (SEQ | | (SEQ ID | | |
| | | ID NO: 46) | | NO: 72) | | |
| | | 13) | | 9) | | |
| | | UGUAACA | | ACACAGG | | |
| | | CCA (SEQ | | UAUUCCA | | |
| | | ID NO: 47) | | GCGAGG | | |
| | | 14) | | SEQ ID | | |
| | | CGGGCCC | | (NO: 73) | | |
| | | CAA (SEQ | | | | |
| | | ID NO: 48) | | | | |
| | | 15) | | | | |
| | | AGGGUCC | | | | |
| | | CCA (SEQ | | | | |
| | | ID NO: 49) | | | | |
| | | 16) | | | | |
| | | GGGUGGG | | | | |
| | | AGA (SEQ | | | | |
| | | ID NO: 50) | | | | |
| | | 17) | | | | |
| | | UCUUCCC | | | | |
| | | CAG (SEQ | | | | |
| | | ID NO: 51) | | | | |
| | | 18) | | | | |
| | | GGCCUGU | | | | |
| | | GAU (SEQ | | | | |
| | | ID NO: 52) | | | | |
| | | 19) | | | | |
| | | GGGCCUA | | | | |
| | | CCA (SEQ | | | | |
| | | ID NO: 53) | | | | |
| | | 20) | | | | |
| | | GAUGCCU | | | | |
| | | GGA (SEQ | | | | |
| | | ID NO: 54) | | | | |
| | | 21) | | | | |
| | | UAGAAAU | | | | |
| | | AGC (SEQ | | | | |
| | | ID NO: 55) | | | | |
| | | 22) | | | | |
| | | CCCAGAU | | | | |
| | | GCG (SEQ | | | | |
| | | ID NO: 56) | | | | |
| | | 23) | | | | |
| | | GGUUUUG | | | | |
| | | GCC (SEQ | | | | |
| | | ID NO: 57) | | | | |
| | | 24) | | | | |
| | | CCACCAA | | | | |
| | | ACU (SEQ | | | | |
| | | ID NO: 58) | | | | |

### 2. Thermal Shift Assay

In an optically sensitive 96 well plate (Biorad), 20 μL PBS buffer containing 4×SYPRO-Orange dye (Thermo Fisher S-6650) were mixed with 5 μL PAP enzyme to obtain a final PAP enzyme concentration of 3 μM. The plate was transferred into a qPCR machine (Biorad CFX96) and a temperature ramp protocol was applied going from 30-90° C. in steps of 0.4° C./min. Fluorescence was measured at each step of 0.4° C. to obtain a melting curve. The inflection point of the melting curve was assumed to be the melting temperature (Tm) of the PAP mutant enzyme.

### 3. Duplex Assay of RNA Synthesis

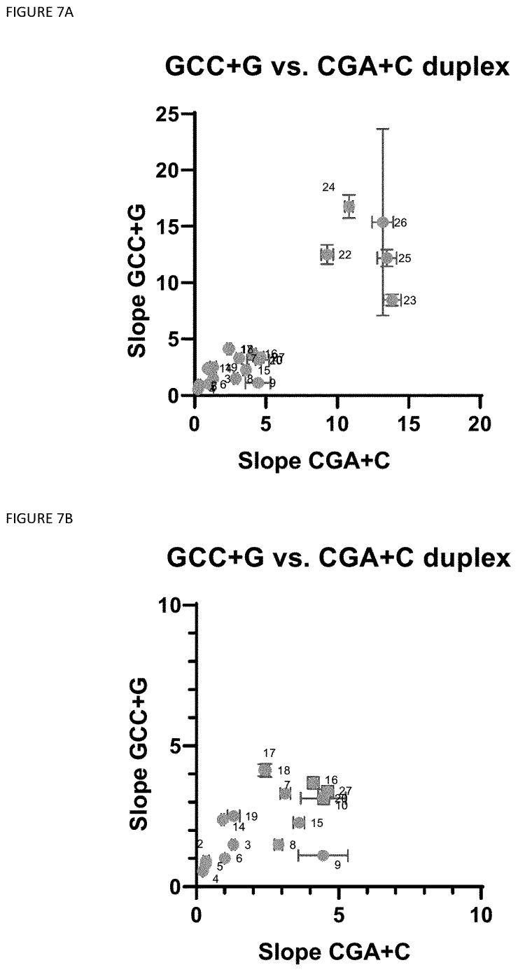

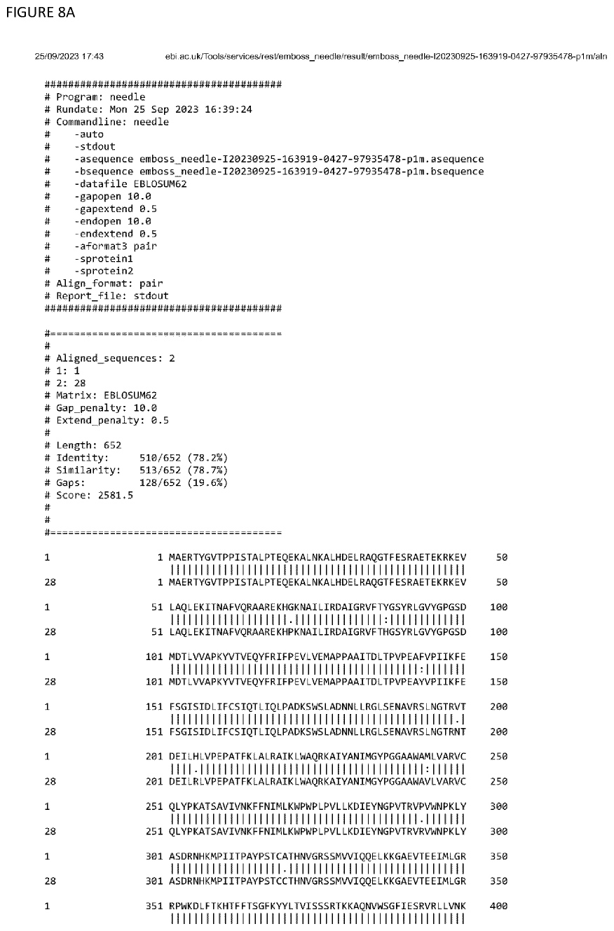

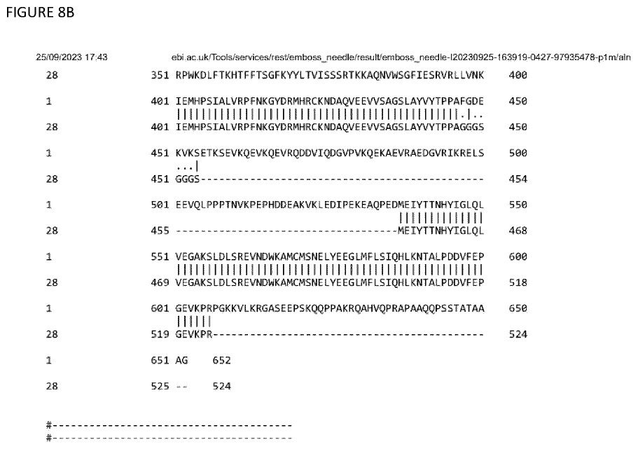

In an optically sensitive 96 well plate, 50 μL PAP (final concentration 0.05 μM) was added. The plate was transferred to a plate reader (BMG Labtech, ClarioStarPlus) preheated at 25° C. A volume of 150 μL of reaction buffer was added to the protein. The fluorescence change was recorded during 30 min. The reaction buffer contained: 25 μM 3′O-azidomethyl-rCTP or 3′O-azidomethyl-rGTP, 1 μM duplex oligo, 10 mM TrisHCl, pH 7.5, 0.1 mM MnCl2, 2×SYBR-Green I (Sigma). Each reaction was carried out in triplicate. To determine the reaction rate of each PAP mutant enzyme the slope of fluorescence increase of the reaction was calculated. The average of three reactions was used as the reaction rate. For PAP mutant enzyme comparisons, the reaction rate of the mutant under assessment was divided by the reaction rate of the variant having the sequence set forth in SEQ ID NO:6 (FIG. 7).

“Duplex GCC+G test” (also referred to as “Test A”) measures the incorporation of 3′O-azidomethyl-rGTP.

“Duplex CGA+C test” (also referred to as “Test B”) measures the incorporation of 3′O-azidomethyl-rCTP.

### 4. Agarose Gel Electrophoresis

RNA oligonucleotides were separated using standard agarose gel electrophoresis and gels were analysed with a GE Typhoon gel imager (as shown for example in Pei Yun Lee et al. J Vis Exp. 2012; (62): 3923).

### 5. Capillary Electrophoresis

RNA oligonucleotides were separated by capillary electrophoresis using an Agilent OligoPro II instrument (https://www.agilent.com/cs/library/brochures/brochure-accurate-assessment-oligonucleotide-purity-oligo-pro-II-5994-0421en-agilent.pdf).

6. PAP Variant Comparison Based on RNA Purities Obtained from Agarose Gel Electrophoresis or Capillary Electrophoresis

As exemplified in FIGS. 2-5, purities of RNA products were determined by: i) counting bands from starting material (called iRNA) to product (called N; eg 10mer, 3mer, 20mer, 40mer) and ii) integrating in software Image lab (Biorad) all bands from iRNA to final product N. In FIGS. 2-5, the % purity of product N vs all other bands is depicted in the table below each lane. To compare mutant activities, the average of all N bands of each mutant was determined and divided by the average of all bands of variants having sequences SEQ ID NO:6 or SEQ ID NO 18 as controls.

### Results

Table 8 below shows the activity of the various mutant poly(A) polymerase enzymes made and tested in the present examples, and their thermal stability as indicated by melting temperature.

SEQ ID NO:1 corresponds to the wild-type PAP sequence from Thermothielavioldes terrestris NRRM 8126 (“Thielavia”).

SEQ ID NO:2 corresponds to the variant described in WO2021/018919.

TABLE 8
results of testing PAP variants of the invention

| | | xfold | xfold | xfold | xfold | xfold | xfold | xfold |
|---|---|---|---|---|---|---|---|---|
| | | SEQ ID | SEQ ID | SEQ ID | SEQ ID | SEQ ID | SEQ ID | SEQ ID |
| | | NO: 6 | NO: 18 | NO: 6 | NO: 6 | NO: 22 | NO: 6 | NO: 6 |
| | | activity; | activity; | activity; | activity; | activity; | activity; | activity; |
| SEQ | | synthesis | synthesis | synthesis | synthesis | synthesis | duplex | duplex |
| ID | | 9x | 6x | 24x | 3x | 3x | assay | assay |
| NO | Tm° C. | 20mers | 4mers | 10mers | 10mers | 10mers | CGA + C | GCC + G |
| 1 | 48.0 | | | | | | | |
| 2 | 46.0 | | | | 0.7 | | 0.4 | 0.9 |
| 3 | 47.6 | | | | 1.0 | | 1.3 | 1.5 |
| 4 | 44.4 | | | | 0.4 | | 0.2 | 0.6 |
| 5 | 47.2 | | | | 0.7 | | 0.3 | 0.8 |
| 6 | 43.0 | 1.0 | | 1.0 | 1.0 | | 1.0 | 1.0 |
| 7 | 44.4 | | | 1.1 | | | 3.1 | 3.3 |
| 8 | 44.4 | | | 1.0 | | | 2.9 | 1.5 |
| 9 | 46.8 | | | 1.0 | | | 4.5 | 1.1 |
| 10 | 50.7 | | | 0.9 | | | 4.5 | 3.2 |
| 14 | 47.2 | | | 1.2 | | | 0.9 | 2.4 |
| 15 | 47.8 | | | 1.4 | | | 3.6 | 2.3 |
| 16 | 51.2 | | | 1.3 | | | 4.1 | 3.7 |
| 17 | 50.4 | | | 1.5 | | | 2.4 | 4.1 |
| 18 | 50.8 | | 1.0 | 1.4 | | | 2.5 | 4.1 |
| 19 | 50.8 | | 1.0 | | | | 1.3 | 2.5 |
| 20 | 51.2 | 1.7 | | | | | 4.5 | 3.1 |
| 21 | 51.6 | 1.8 | | | | | | |
| 22 | 51.2 | 2.1 | 1.2 | | | 1.0 | 9.3 | 12.5 |
| 23 | 51.2 | | | | | 1.0 | 13.9 | 8.5 |
| 24 | 51.2 | | | | | 1.1 | 13.5 | 12.2 |
| 26 | 55.2 | | | | | 1.1 | 10.8 | 16.8 |
| 25 | 51.2 | | | | | 1.1 | 13.2 | 15.4 |
| 27 | 55.6 | | | | | 0.4 | 4.6 | 3.4 |
| 28 | 55.3 | | | | | 1.2 | | |

The results show that the mutant PAP enzymes of the present invention (shown in full at SEQ ID NO:3, and 6 to 28 herein) display a trend towards a higher stability, as shown by a general increase in the melting temperature compared to known mutant having SEQ ID NO:2. The results further show that most mutant PAP enzymes generated herein had at least comparable activity to the known variant having the sequence SEQ ID NO:2 when synthesising different short oligonucleotides, with a large number of mutant PAP enzymes showing an increase in activity compared to said known variant. Some mutants consistently showed over 10× the activity of SEQ ID NO:2 in the duplex assay, with many showing at least 2× the activity of SEQ ID NO:2 in this assay. Since the variant having the sequence as set forth in SEQ ID NO:2 had previously been shown to have improved activity compared to the wild-type sequence having SEQ ID NO:1, the variants according to the invention display improved activity compared to that of the wild-type PAP enzyme. Therefore, the mutant PAP enzymes of the invention have been shown to have improved stability and activity compared to PAP enzymes currently available for conducting template free polynucleotide synthesis in the art.

This improved stability and activity is observed for mutant PAP enzymes whose amino acid sequence differs quite significantly from the wild-type PAP enzyme having SEQ ID NO:1.

For instance, the PAP variant having the sequence set forth in SEQ ID NO:28 comprises mutations at residues G71, Y87, F143, V199, H205, V240, M244, P293, M318, A320 and A410 of SEQ ID NO:1, a “2GS” flexible loop replacement; and a C-terminal truncation. It possesses a sequence identity of 78.2% with SEQ ID NO:1 over the full length of SEQ ID NO:1 (see FIG. 8).

### Example 2: Further PAP Mutants According to the Invention

Further variants of the WT PAP enzyme having the SEQ ID NO:1 were constructed and tested under the same conditions as in Example 1. These variants have the sequences as set forth in SEQ ID NO:110 to SEQ ID NO:130.

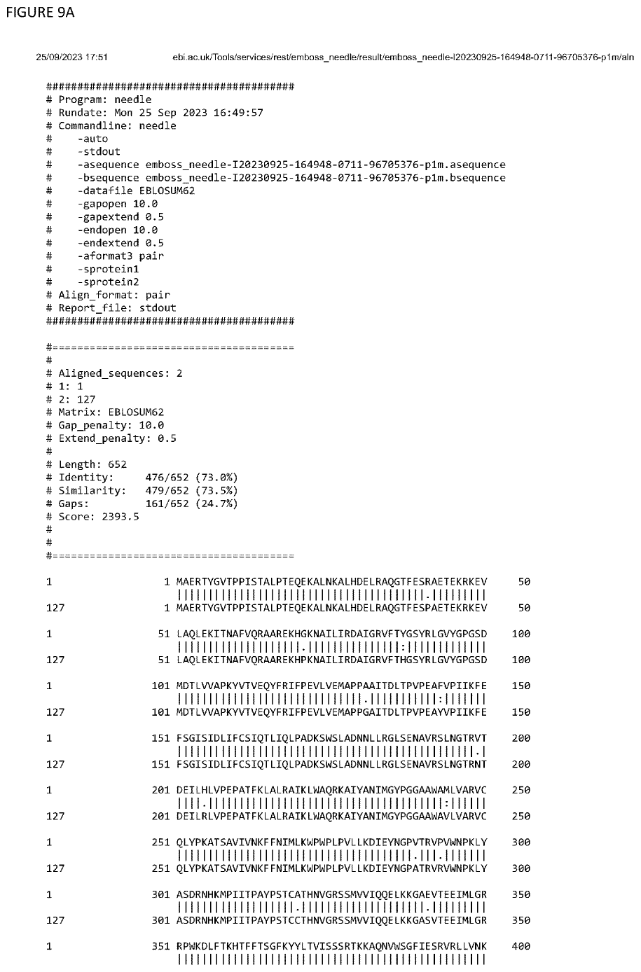

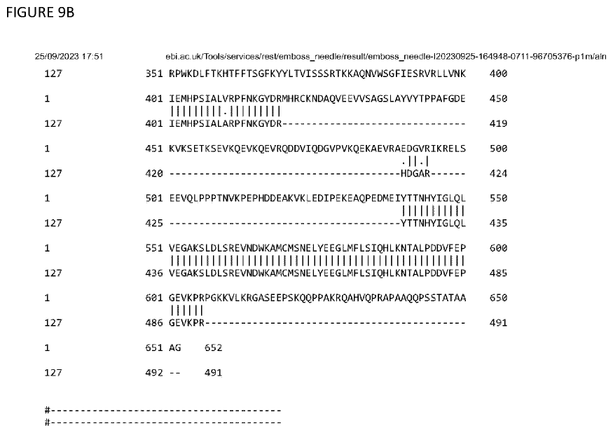

For instance, the PAP variant having the sequence set forth in SEQ ID NO:127 comprises mutations at residues R41, G71, Y87, A131, F143, V199, H205, V240, M244, V289, P293, M318, A320 and E341 of SEQ ID NO:1, a “HDGAR” flexible loop replacement; and a C-terminal truncation. It possesses a sequence identity of 73% with SEQ ID NO:1 over the full length of SEQ ID NO:1 (see FIG. 9).

In addition, the duplex assay of RNA synthesis described in Example 1 above was performed with a variety of different protected rNTPs:
3′-O-azidomethyl-rGTP (AM-rGTP);
3′-O-azidomethyl-2′-O-methyl-rGTP (AM-OMrGTP)
3′-O-azidomethyl-2′-fluoro-rGTP (AM-FrGTP)
3′-O-azidomethyl-rCTP (AM-rCTP);
3′-O-azidomethyl-2′-O-methyl-rCTP (AM-OMrCTP)
3′-O-azidomethyl-2′-fluoro-rCTP (AM-FrCTP).

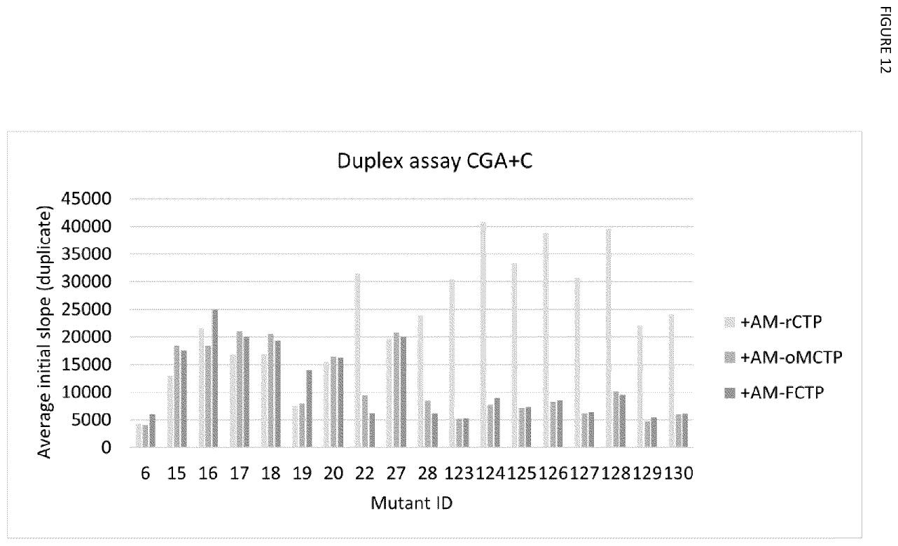

The results of these duplex assays as well as the melting temperature determined by thermal shift assay are presented in Table 9 and in FIGS. 11 and 12.

TABLE 9
results of testing PAP variants of the invention for stability and for activity

| | | | | | | | | Error |
|---|---|---|---|---|---|---|---|---|
| SEQ | | | | | | | | rate |
| ID | Tm | +AM- | +AM- | +AM- | +AM- | +AM- | +AM- | (from |
| NO: | (° C.) | rGTP | oMGTP | FGTP | rCTP | oMCTP | FCTP | NGS) |
| 6 | 43 | 2757 | 264 | 1051 | 4360 | 4074 | 5981 | |
| 15 | 47.8 | 2651 | 808 | 2092 | 13065 | 18499 | 17560 | |
| 16 | 51.2 | 3660 | 4195 | 8374 | 21630 | 18514 | 24959 | |
| 17 | 50.4 | 3066 | 3578 | 3391 | 16819 | 21046 | 19953 | |
| 18 | 50.8 | 3792 | 4009 | 5206 | 16918 | 20609 | 19369 | |
| 19 | 50.8 | 8577 | 4288 | 12697 | 7605 | 7945 | 14032 | |
| 20 | 51.2 | 3103 | 2595 | 3345 | 15497 | 16489 | 16254 | |
| 22 | 51.2 | 28943 | 1010 | 4541 | 31498 | 9422 | 6271 | |
| 27 | 55.00 | 2752 | 3691 | 2729 | 19680 | 20833 | 20014 | |
| 28 | 55.3 | 15414 | 710 | 2117 | 23956 | 8506 | 6134 | 3.18 |
| 123 | 54.40 | 14262 | 237 | 1796 | 30463 | 5300 | 5367 | 2.89 |
| 124 | 55.60 | 22109 | 347 | 2892 | 40910 | 7705 | 8901 | 3.16 |
| 125 | 55.20 | 17361 | 355 | 2860 | 33361 | 7245 | 7335 | 4.08 |
| 126 | 55.80 | 21607 | 491 | 3178 | 38797 | 8348 | 8565 | 3.2 |
| 127 | 54.80 | 17071 | 312 | 3001 | 30804 | 6134 | 6454 | 2.62 |
| 128 | 55.6 | 28700 | 1168 | 5310 | 39533 | 10213 | 9530 | 2.96 |
| 129 | 55.2 | 21541 | 530 | 3830 | 22083 | 4777 | 5482 | 2.91 |
| 130 | 55.2 | 25717 | 698 | 4680 | 24182 | 6039 | 6061 | 2.75 |

Table 9 shows that the variants having the sequences set forth in SEQ ID NO: 123 to 130 also having a higher stability compared to the variant known in the art. More specifically these variants all exhibit a melting temperature above 54° C.

In addition, they all display high activity for the incorporation of rGTP or rCTP, even higher than the variants disclosed in Example 1 above.

Furthermore, the variants according to the invention display PAP activity even when modified rNTPs such as O-methyl-rNTPs and fluoro-rNTPs are used. In particular, the PAP variants

### Example 3: PAP Variants Derived from Wild-Type Chaetomium PAP

In this example, the wild-type PAP enzyme from Chaetomium was used. This enzyme has the sequence set forth in SEQ ID NO:76 (Chaetomium thermophilum var thermophilum DSM 1495).

A first variant of this sequence was made by carrying out a C-terminal truncation and a replacement of the flexible loop by a “HDGAR” loop. This variant has the sequence as set forth in SEQ ID NO:131.

Then, various point mutations or combinations of points mutations were carried out, as indicated in Table 10 below. The effect of these point mutations or combinations or mutations on the activity and the stability of the enzyme was observed.

The variant SEQ ID NO:132 is derived from SEQ ID NO:131 and comprises a mutation at residue corresponding to Y87 in SEQ ID NO:1.

The variant SEQ ID NO:133 is derived from SEQ ID NO:131 and comprises a mutation at residue corresponding to F143 in SEQ ID NO:1.

The variant SEQ ID NO:134 is derived from SEQ ID NO:131 and comprises a mutation at residue corresponding to K148 in SEQ ID NO:1.

The variant SEQ ID NO:135 is derived from SEQ ID NO:131 and comprises a mutation at residue corresponding to V199 in SEQ ID NO:1.

The variant SEQ ID NO:136 is derived from to SEQ ID NO:131 and comprises a mutation at residue corresponding to N205 in SEQ ID NO:1.

The variant SEQ ID NO:137 is derived from to SEQ ID NO:131 and comprises a mutation at residue corresponding to V240 in SEQ ID NO:1.

The variant SEQ ID NO:138 is derived from to SEQ ID NO:131 and comprises a mutation at residue corresponding to M244 in SEQ ID NO:1.

The variant SEQ ID NO:139 is derived from to SEQ ID NO:131 and comprises a mutation at residue corresponding to A293 in SEQ ID NO:1.

The variant SEQ ID NO:140 is derived from to SEQ ID NO:131 and comprises a mutation at residue corresponding to M318 in SEQ ID NO:1.

The variant SEQ ID NO:141 is derived from to SEQ ID NO:131 and comprises a mutation at residue corresponding to A320 in SEQ ID NO:1.

The variant SEQ ID NO:142 is derived from to SEQ ID NO:131 and comprises a mutation at residues corresponding to V199, V240, M244 and M318 in SEQ ID NO:1.

The variant SEQ ID NO:143 is derived from to SEQ ID NO:131 and comprises a mutation at residues corresponding to F143, V199, N205, V240, M244, A293, M318 and A320 in SEQ ID NO:1.

The variant SEQ ID NO:144 is derived from to SEQ ID NO:131 and comprises a mutation at residue corresponding to Y87, F143, V199, N205, V240, M244, A293, M318 and A320 in SEQ ID NO:1.

The variant SEQ ID NO:145 is derived from to SEQ ID NO:131 and comprises a mutation at residue corresponding to Y87, F143, K148, V199, N205, V240, M244, A293, M318 and A320 in in SEQ ID NO:1.

The activity of said variants in the “duplex GCC+G” assay described in Example 1 was compared to that of the PAP variant SEQ ID NO:6 and expressed as the ratio “activity of variant/activity of SEQ ID NO:6”.

The results of this activity test and the melting temperatures determined by TSA are presented in Table 10 below.

TABLE 10
results of testing PAP variants of the invention in Chaetomium.
A. melting temperatures. B. activity ratio compared to SEQ ID NO: 6
A. Melting temperatures in ° C.

| Thielavia | | | | | Chaetomium | | | | | |
|---|---|---|---|---|---|---|---|---|---|---|
| 123 | 22* | 18* | 6* | | SEQ ID NO: | | 142 | 143 | 144 | 145 |
| 54.4 | 51.2 | 50.8 | 43 | V199N | 135 | 50.8 | 51.6 | 54 | 53.8 | 52.4 |
| | | | | V240N | 137 | 47.4 | | | | |
| | | | | M244V | 138 | 50.0 | | | | |
| | | | | M318T | 140 | 47.6 | | | | |

| F143Y | 133 | 48.4 |
|---|---|---|
| Q293R | 139 | 48.8 |
| A320C | 141 | 48.2 |
| N205R | 136 | |

| | | |
|---|---|---|
| Y87H | 132 | 48 |

| | | |
|---|---|---|
| K148N | 134 | 46.8 |

| | | | | | | |
|---|---|---|---|---|---|---|
| yes | No* | No* | No* | Loop + | 131 | 46.4 |

| | |
|---|---|
| Cter | |
| WT | 76 |

B. Activity ratios in duplex GCC + G (compared to SEQ ID NO: 6)

| Thielavia | | | | | Chaetomium | | | | | |
|---|---|---|---|---|---|---|---|---|---|---|
| 123 | 22* | 18* | 6* | | SEQ ID NO: | | 142 | 143 | 144 | 145 |
| 2.3 | 2.1 | 1.9 | 1.0 | V199N | 135 | 0.1 | 0.4 | 1.3 | 1.5 | 2.1 |
| | | | | V240N | 137 | 0.4 | | | | |
| | | | | M244V | 138 | 0.1 | | | | |
| | | | | M318T | 140 | 0.1 | | | | |

| F143Y | 133 | 0.0 |
|---|---|---|
| Q293R | 139 | 0.3 |
| A320C | 141 | 0.3 |
| N205R | 136 | |
| Y87H | 132 | 0.3 |
| K148N | 134 | 0.4 |
| Loop + | 131 | 0.1 |
| Cter | | |

| | | |
|---|---|---|
| WT | 76 | 0.1 |

The sequences from Thielavia indicated by an asterisk (*) do not comprise the loop replacement nor the C-terminal truncation.

These results demonstrate that, starting from a given species in the Chaetomium family, carrying out the point mutations at residues corresponding to V199 V240, and M318 in Thielavia also improves the activity of the PAP enzyme compared to wild-type sequence and improves the stability of the enzyme (as reflected by increased Tm).

Further mutations in residues corresponding to residues F143, A293, A320, N205, Y87 and K148 further improve the activity and the stability.

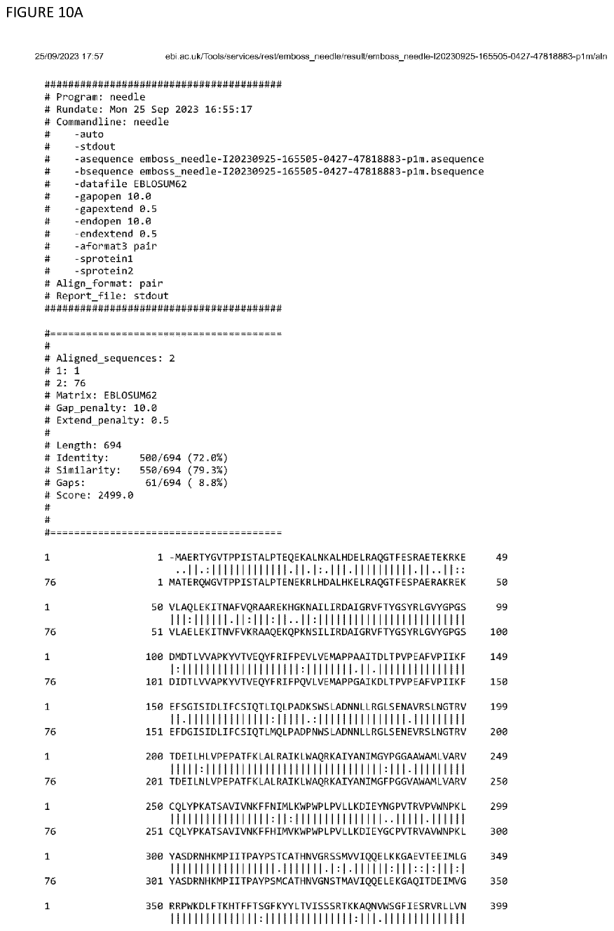

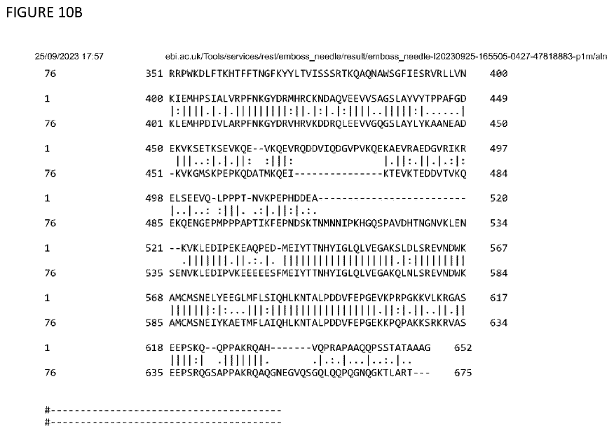

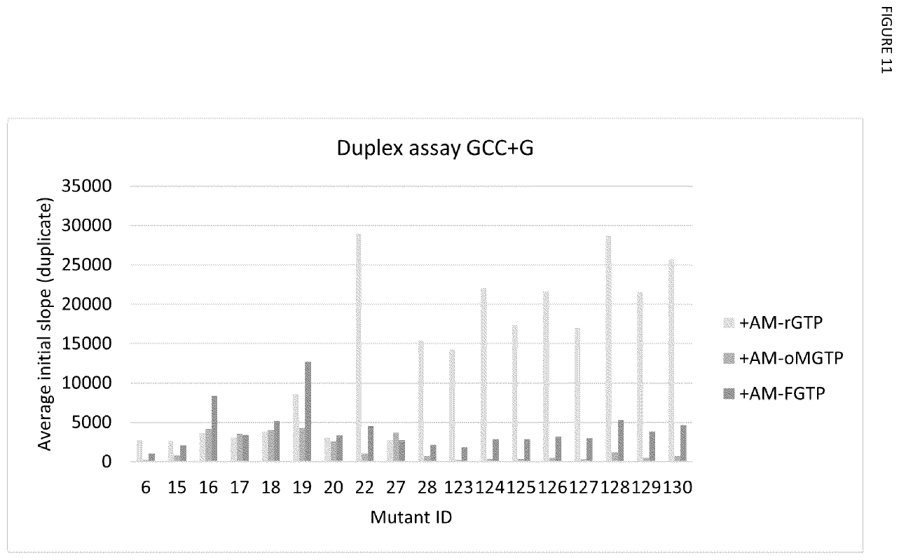

The wild-type sequence of the PAP enzyme in Thielavia (SEQ ID NO:1) and in Chaetomium (SEQ ID NO:76) display only 72% identity over the entire length of SEQ ID NO:1 (see FIG. 10). Hence, these results show that carrying out the mutations described in the present invention has similar effects in different species, even in species displaying around 70% identity.

### Example 4: PAP Variants Derived from Phaeoacremonium and Ophiostoma

In this example, the PAP enzymes from Phaeoacremonium and Ophiostoma were modified according to the invention and compared to PAP variants from Thielavia. The wild-type sequences for these enzymes have the sequences set forth in SEQ ID NO:149 (Phaeoacremonium) and SEQ ID NO: 153 (Ophiostoma).

First, tagged versions of the enzymes were produced, by introducing a 6-His tag at the C-terminal end (SEQ ID NO 146, 150 and 154 respectively for Thielavia, Phaeoacremonium and Ophiostoma).

Then, a “HDGAR loop and C-terminal truncation” variant was made for each enzyme, by carrying out a flexible loop replacement by a HDGAR loop and a C-terminal truncation up to the residue corresponding to residue 606 in SEQ ID NO:1. (SEQ ID NO 147, 151 and 155 respectively for Thielavia, Phaeoacremonium and Ophiostoma).

Finally, the following mutations were made, in the residues corresponding to the following residues in SEQ ID NO:1: R41, G71, Y87 A131, F143, V199, H205, V240, M244, V289, P299 M318, A320 and E341 (SEQ ID NO 148, 152 and 156 respectively for Thielavia, Phaeoacremonium and Ophiostoma).

The variants were produced as described above and were analysed for their production yield according to the following protocol. For the SDS PAGE: 2 μL of PAP variant in elution buffer were mixed with 10 μL 2× Laemmli buffer and 8 μL water. Samples were applied to a pre-stained SDS polyacrylamide gel (Biorad 4-15% Criterion TGX; #5671085) and run in standard SDS PAGE running buffer. Gels were analysed using a Gel reader. The intensity of each band was normalised to the intensity of the Thielavia variant having SEQ ID NO 148.

The results are presented in Table 11 below:

| | Thielavia | | | Phaeoacremonium | | | Ophiostoma | | |
|---|---|---|---|---|---|---|---|---|---|
| | | | Loop + | | | Loop + | | | Loop + |
| | | | Cter + | | | Cter + | | | Cter + |
| | | Loop + | point | | Loop + | point | | Loop + | point |
| | WT | Cter | mutations | WT | Cter | mutations | WT | Cter | mutations |
| SEQ ID | 146 | 147 | 148 | 150 | 151 | 152 | 154 | 155 | 156 |
| NO: | | | | | | | | | |
| PAP band | 8 | 19 | 100 | 23 | 46 | 81 | 0 | 3 | 6 |
| intensity | | | | | | | | | |
| vs SEQ ID | | | | | | | | | |
| 148% | | | | | | | | | |
| Sequence | | | 100 | | | 70 | | | 67 |
| identity | | | | | | | | | |
| vs SEQ ID | | | | | | | | | |
| NO: 148 | | | | | | | | | |

The results demonstrate that the production yield is increased by a factor of around 2 upon replacement of the flexible loop by a HDGAR loop and C-terminal truncation in all three species. These conclusions are true for PAP enzymes derived from very different organisms. For instance, SEQ ID 149 shares only 55.8% identity with SEQ ID NO:1 over the entire length of SEQ ID NO:1. Similarly, SEQ ID NO 153 shares 52.1% identity with SEQ ID NO:1 over the entire length of SEQ ID NO:1.

The yield is further increased when the various point mutations are introduced.

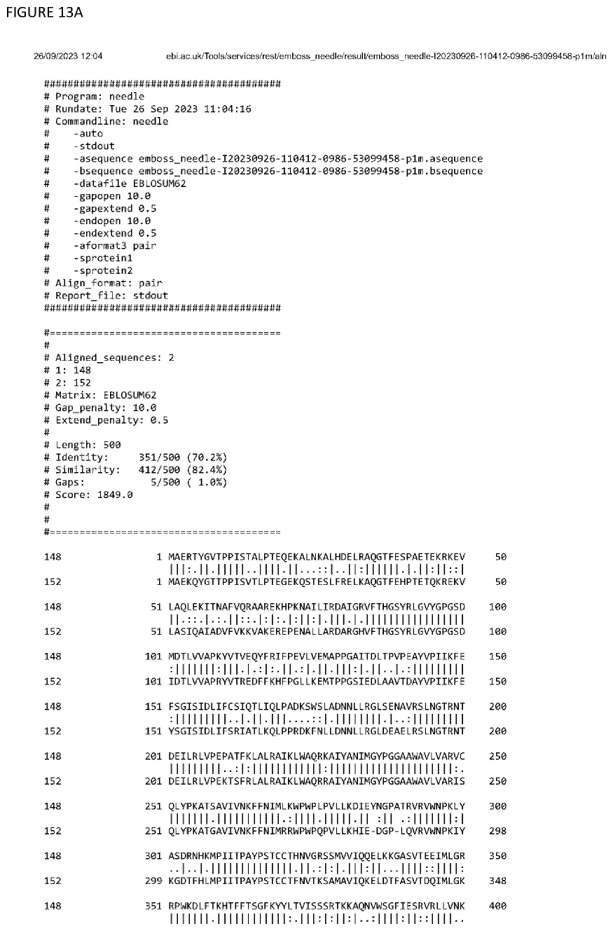

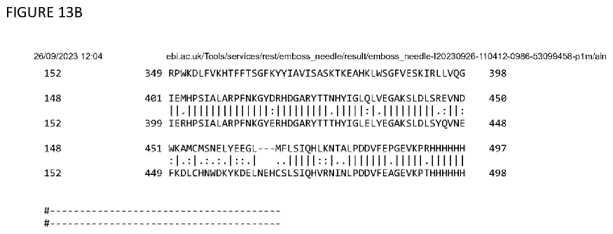

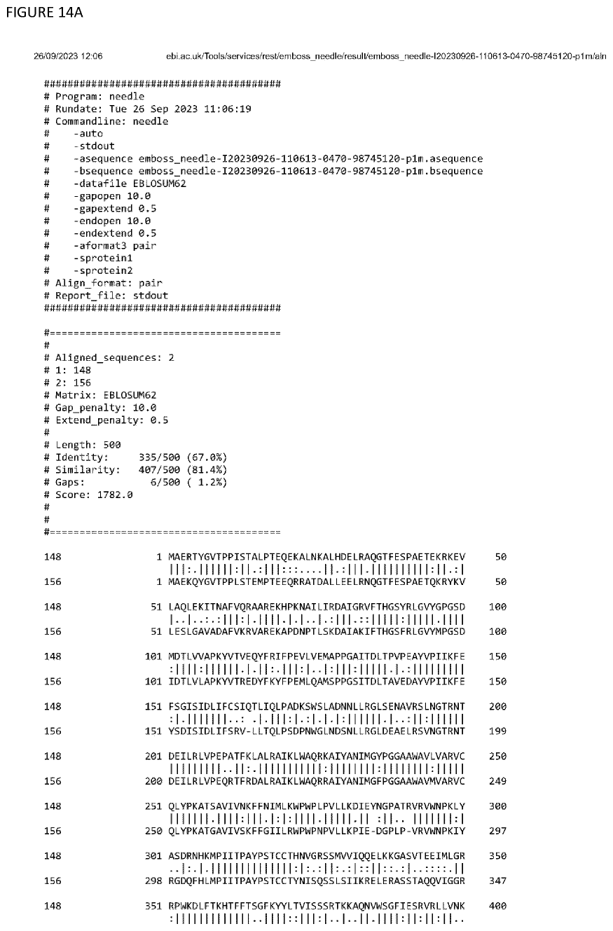

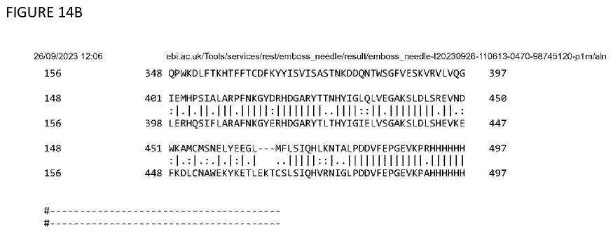

The effect of the point mutations is also very strong, since it can be observed in PAP enzymes derived from relatively distant species. Indeed, the sequence identity of SEQ ID NO: 152 and SEQ ID NO:156 with SEQ ID NO:148 (which represents the functional fragment of SEQ ID NO:1 with mutations at residues R41, G71, Y87 A131, F143, V199, H205, V240, M244, V289, P299 M318, A320 and E341) is respectively 70 and 67% (see FIGS. 13 and 14).

1. A poly(A) polymerase variant comprising an amino acid sequence having at least 70% identity to SEQ ID NO:1 or a functional fragment thereof, and comprising mutations at residues V199, V240, and M318 of SEQ ID NO:1 or at functionally equivalent residues thereto.

2. The poly(A) polymerase variant of claim 1, wherein the enzyme further comprises one or more mutations at residues selected from: G71, Y87, A131, F143, N195, H205, P208, M244, P293, A320, Q334, K337, A410, E574, and P643 of SEQ ID NO:1, or at functionally equivalent residues thereto.

3. The poly(A) polymerase variant of claim 1, wherein the enzyme further comprises a mutation at residue: P293 of SEQ ID NO:1, or at a functionally equivalent residue thereto or at residue: M244 of SEQ ID NO:1, or at a functionally equivalent residue thereto.

4. The poly(A) polymerase variant of claim 1, wherein the enzyme further comprises one or more mutations at residues selected from R41, V289, E341, K381, S387, 1401, K415, M571 and E577 of SEQ ID NO:1:

5. The poly(A) polymerase variant of claim 1, wherein the mutations are substitution mutations.

6. The poly(A) polymerase variant of claim 5,
wherein the substitution mutation at residue V199, or a functionally equivalent residue thereto is V199N or V199T, or
wherein the substitution mutation at residue V240, or a functionally equivalent residue thereto is V240A, or
wherein the substitution mutation at residue M318, or a functionally equivalent residue thereto is M318T.

7. The poly(A) polymerase variant of claim 6,
wherein the substitution mutation at residue G71, or a functionally equivalent residue thereto is G71P, or
wherein the substitution mutation at residue Y87, or a functionally equivalent residue thereto is Y87H, or
wherein the substitution mutation at residue A131, or a functionally equivalent residue thereto is A131G, or
wherein the substitution mutation at residue F143, or a functionally equivalent residue thereto is F143Y, or
wherein the substitution mutation at residue N195, or a functionally equivalent residue thereto is N195S, or
wherein the substitution mutation at residue H205, or a functionally equivalent residue thereto is H205R or H205K, or
wherein the substitution mutation at residue P208, or a functionally equivalent residue thereto is P208H, or
wherein the substitution mutation at residue M244, or a functionally equivalent residue thereto is M244V, or
wherein the substitution mutation at residue P293, a functionally equivalent residue thereto is P293R, or
wherein the substitution mutation at residue A320, or a functionally equivalent residue thereto is A320T or A320C, or
wherein the substitution mutation at residue Q334, or a functionally equivalent residue thereto is Q334K, or
wherein the substitution mutation at residue A410, or a functionally equivalent residue thereto is A410V, or
wherein the substitution mutation at residue P643, or a functionally equivalent residue thereto is P643A, or wherein the substitution mutation at residue R41 or a functionally equivalent residue thereto is R41P, or
wherein the substitution mutation at residue V289 or a functionally equivalent residue thereto is V289A, or
wherein the substitution mutation at residue E341 or a functionally equivalent residue thereto is E341S, or
wherein the substitution mutation at residue K381 or a functionally equivalent residue thereto is K381Q, or
wherein the substitution mutation at residue S387 or a functionally equivalent residue thereto is S387R, or
wherein the substitution mutation at residue I401 or a functionally equivalent residue thereto is I401L, or
wherein the substitution mutation at residue K415 or a functionally equivalent residue thereto is K415Q, or
wherein the substitution mutation at residue M571 or a functionally equivalent residue thereto is M571K, or
wherein the substitution mutation at residue E577 or a functionally equivalent residue thereto is E577R.

8. The poly(A) polymerase variant of claim 1, wherein the enzyme further comprises a flexible loop replacement or a C-terminal truncation.

9. The poly(A) polymerase variant of claim 8, wherein the flexible loop replacement is an ancestral loop (SEQ ID NO:32 or SEQ ID NO:33) or a 2GS loop (GGGSGGGS SEQ ID NO:31) or a HDGAR loop (SEQ ID NO: 157).

10. The poly(A) polymerase variant of claim 8, wherein the C-terminal truncation is up to residue 606 of SEQ ID NO:1 or a functionally equivalent residue thereto.

11. The poly(A) polymerase variant of claim 1 wherein the enzyme comprises an amino acid sequence having at least at least 90% identity to SEQ ID NO:1.

12. The poly(A) polymerase variant of claim 1, wherein the enzyme comprises a sequence according to any one of SEQ ID NOs: 3, 6-28, 110-130, 142-145, 151-152, 155-156 or 158-159, or a sequence having at least 90% identity thereto.

13. A method of synthesizing a polynucleotide comprising:
(a) providing a nucleic acid initiator;
(b) contacting the nucleic acid initiator with the poly(A) polymerase variant of claim 1, and a protected nucleotide under suitable conditions to incorporate the protected nucleotide into the nucleic acid initiator and form an elongated nucleic acid fragment;
(c) deprotecting the elongated nucleic acid fragment;
(d) repeating steps (b) and (c) on the elongated nucleic acid fragment until the polynucleotide is formed

14. The polynucleotide obtained by the method of claim 13.

15. A kit comprising the poly(A) polymerase variant of claim 1, and one or more reagents for synthesising a polynucleotide.


SEQUENCE LISTING (159 sequences)

SEQ ID NO: 1 - protein, 650 aa, Thermothielavioides terrestris
MAERTYGVTPPISTALPTEQEKALNKALHDELRAQGTFESRAETEKRKEVLAQLEKITNAFVQRAAREKHGKNAILIRDAIGRVFTYGSYRLGVYGPGSDMDTLVVAPKYVTVEQYFRIFPEVLVEMAPPAAITDLTPVPEAFVPIIKFEFSGISIDLIFCSIQTLIQLPADKSWSLADNNLLRGLSENAVRSLNGTRVTDEILHLVPEPATFKLALRAIKLWAQRKAIYANIMGYPGGVAWAMLVARVCQLYPKATSAVIVNKFFNIMLKWPWPLPVLLKDIEYNGPVTRVPVWNPKLYASDRNHKMPIITPAYPSMCATHNVGRSSMVVIQQELKKGAEVTEEIMLGRRPWKDLFTKHTFFTSGFKYYLTVISSSRTKKAQNVWSGFIESRVRLLVNKIEMHPSIALARPFNKGYDRMHRCKNDAQVEEVVSAGSLAYVYTPPAFGDEKVKSETKSEVKQEVKQEVRQDDVIQDGVPVKQEKAEVRAEDGVRIKRELSEEVQLPPPTNVKPEPHDDEAKVKLEDIPEKEAQPEDMEIYTTNHYIGLQLVEGAKSLDLSREVNDWKAMCMSNELYEEGLMFLSIQHLKNTALPDDVFEPGEVKPRPGKKVLKRGASEEPSKQQPPAKRQAHVQPRAPAAQQPSSTATAA

SEQ ID NO: 2 - protein, 652 aa, synthetic construct
MAERTYGVTPPISTALPTEQEKALNKALHDELRAQGTFESRAETEKRKEVLAQLEKITNAFVQRAAREKHGKNAILIRDAIGRVFTYGSYRLGVYGPGSDMDTLVVAPKYVTVEQYFRIFPEVLVEMAPPAAITDLTPVPEAFVPIIKFEFSGISIDLIFCSIQTLIQLPADKSWSLADNNLLRGLSENAVRSLNGTRVTDEILHLVPEPATFKLALRAIKLWAQRKAIYANIMGYPGGAAWAMLVARVCQLYPKATSAVIVNKFFNIMLKWPWPLPVLLKDIEYNGPVTRVPVWNPKLYASDRNHKMPIITPAYPSTCATHNVGRSSMVVIQQELKKGAEVTEEIMLGRRPWKDLFTKHTFFTSGFKYYLTVISSSRTKKAQNVWSGFIESRVRLLVNKIEMHPSIALVRPFNKGYDRMHRCKNDAQVEEVVSAGSLAYVYTPPAFGDEKVKSETKSEVKQEVKQEVRQDDVIQDGVPVKQEKAEVRAEDGVRIKRELSEEVQLPPPTNVKPEPHDDEAKVKLEDIPEKEAQPEDMEIYTTNHYIGLQLVEGAKSLDLSREVNDWKAMCMSNELYEEGLMFLSIQHLKNTALPDDVFEPGEVKPRPGKKVLKRGASEEPSKQQPPAKRQAHVQPRAPAAQQPSSTATAAAG

SEQ ID NO: 3 - protein, 652 aa, synthetic construct
MAERTYGVTPPISTALPTEQEKALNKALHDELRAQGTFESRAETEKRKEVLAQLEKITNAFVQRAAREKHGKNAILIRDAIGRVFTYGSYRLGVYGPGSDMDTLVVAPKYVTVEQYFRIFPEVLVEMAPPAAITDLTPVPEAFVPIIKFEFSGISIDLIFCSIQTLIQLPADKSWSLADNNLLRGLSENAVRSLNGTRNTDEILHLVPEPATFKLALRAIKLWAQRKAIYANIMGYPGGAAWAMLVARVCQLYPKATSAVIVNKFFNIMLKWPWPLPVLLKDIEYNGPVTRVPVWNPKLYASDRNHKMPIITPAYPSTCATHNVGRSSMVVIQQELKKGAEVTEEIMLGRRPWKDLFTKHTFFTSGFKYYLTVISSSRTKKAQNVWSGFIESRVRLLVNKIEMHPSIALVRPFNKGYDRMHRCKNDAQVEEVVSAGSLAYVYTPPAFGDEKVKSETKSEVKQEVKQEVRQDDVIQDGVPVKQEKAEVRAEDGVRIKRELSEEVQLPPPTNVKPEPHDDEAKVKLEDIPEKEAQPEDMEIYTTNHYIGLQLVEGAKSLDLSREVNDWKAMCMSNELYEEGLMFLSIQHLKNTALPDDVFEPGEVKPRPGKKVLKRGASEEPSKQQPPAKRQAHVQPRAPAAQQASSTATAAAG

SEQ ID NO: 4 - protein, 652 aa, synthetic construct
MAERTYGVTPPISTALPTEQEKALNKALHDELRAQGTFESRAETEKRKEVLAQLEKITNAFVQRAAREKHGKNAILIRDAIGRVFTYGSYRLGVYGPGSDMDTLVVAPKYVTVEQYFRIFPEVLVEMAPPAAITDLTPVPEAFVPIIKFEFSGISIDLIFCSIQTLIQLPADKSWSLADNNLLRGLSENAVRSLNGTRVTDEILHLVHEPATFKLALRAIKLWAQRKAIYANIMGYPGGAAWAMLVARVCQLYPKATSAVIVNKFFNIMLKWPWPLPVLLKDIEYNGPVTRVPVWNPKLYASDRNHKMPIITPAYPSTCATHNVGRSSMVVIQQELKKGAEVTEEIMLGRRPWKDLFTKHTFFTSGFKYYLTVISSSRTKKAQNVWSGFIESRVRLLVNKIEMHPSIALVRPFNKGYDRMHRCKNDAQVEEVVSAGSLAYVYTPPAFGDEKVKSETKSEVKQEVKQEVRQDDVIQDGVPVKQEKAEVRAEDGVRIKRELSEEVQLPPPTNVKPEPHDDEAKVKLEDIPEKEAQPEDMEIYTTNHYIGLQLVEGAKSLDLSREVNDWKAMCMSNELYEEGLMFLSIQHLKNTALPDDVFEPGEVKPRPGKKVLKRGASEEPSKQQPPAKRQAHVQPRAPAAQQASSTATAAAG

SEQ ID NO: 5 - protein, 652 aa, synthetic construct
MAERTYGVTPPISTALPTEQEKALNKALHDELRAQGTFESRAETEKRKEVLAQLEKITNAFVQRAAREKHGKNAILIRDAIGRVFTYGSYRLGVYGPGSDMDTLVVAPKYVTVEQYFRIFPEVLVEMAPPAAITDLTPVPEAFVPIIKFEFSGISIDLIFCSIQTLIQLPADKSWSLADNNLLRGLSENAVRSLNGTRVTDEILHLVPEPATFKLALRAIKLWAQRKAIYANIMGYPGGAAWAVLVARVCQLYPKATSAVIVNKFFNIMLKWPWPLPVLLKDIEYNGPVTRVPVWNPKLYASDRNHKMPIITPAYPSTCATHNVGRSSMVVIQQELKKGAEVTEEIMLGRRPWKDLFTKHTFFTSGFKYYLTVISSSRTKKAQNVWSGFIESRVRLLVNKIEMHPSIALVRPFNKGYDRMHRCKNDAQVEEVVSAGSLAYVYTPPAFGDEKVKSETKSEVKQEVKQEVRQDDVIQDGVPVKQEKAEVRAEDGVRIKRELSEEVQLPPPTNVKPEPHDDEAKVKLEDIPEKEAQPEDMEIYTTNHYIGLQLVEGAKSLDLSREVNDWKAMCMSNELYEEGLMFLSIQHLKNTALPDDVFEPGEVKPRPGKKVLKRGASEEPSKQQPPAKRQAHVQPRAPAAQQASSTATAAAG

SEQ ID NO: 6 - protein, 652 aa, synthetic construct
MAERTYGVTPPISTALPTEQEKALNKALHDELRAQGTFESRAETEKRKEVLAQLEKITNAFVQRAAREKHGKNAILIRDAIGRVFTYGSYRLGVYGPGSDMDTLVVAPKYVTVEQYFRIFPEVLVEMAPPAAITDLTPVPEAFVPIIKFEFSGISIDLIFCSIQTLIQLPADKSWSLADNNLLRGLSENAVRSLNGTRNTDEILHLVHEPATFKLALRAIKLWAQRKAIYANIMGYPGGAAWAVLVARVCQLYPKATSAVIVNKFFNIMLKWPWPLPVLLKDIEYNGPVTRVPVWNPKLYASDRNHKMPIITPAYPSTCATHNVGRSSMVVIQQELKKGAEVTEEIMLGRRPWKDLFTKHTFFTSGFKYYLTVISSSRTKKAQNVWSGFIESRVRLLVNKIEMHPSIALVRPFNKGYDRMHRCKNDAQVEEVVSAGSLAYVYTPPAFGDEKVKSETKSEVKQEVKQEVRQDDVIQDGVPVKQEKAEVRAEDGVRIKRELSEEVQLPPPTNVKPEPHDDEAKVKLEDIPEKEAQPEDMEIYTTNHYIGLQLVEGAKSLDLSREVNDWKAMCMSNELYEEGLMFLSIQHLKNTALPDDVFEPGEVKPRPGKKVLKRGASEEPSKQQPPAKRQAHVQPRAPAAQQASSTATAAAG

SEQ ID NO: 7 - protein, 652 aa, synthetic construct
MAERTYGVTPPISTALPTEQEKALNKALHDELRAQGTFESRAETEKRKEVLAQLEKITNAFVQRAAREKHGKNAILIRDAIGRVFTYGSYRLGVYGPGSDMDTLVVAPKYVTVEQYFRIFPEVLVEMAPPAAITDLTPVPEAFVPIIKFEFSGISIDLIFCSIQTLIQLPADKSWSLADNNLLRGLSENAVRSLNGTRNTDEILHLVHEPATFKLALRAIKLWAQRKAIYANIMGYPGGAAWAVLVARVCQLYPKATSAVIVNKFFNIMLKWPWPLPVLLKDIEYNGPVTRVRVWNPKLYASDRNHKMPIITPAYPSTCATHNVGRSSMVVIQQELKKGAEVTEEIMLGRRPWKDLFTKHTFFTSGFKYYLTVISSSRTKKAQNVWSGFIESRVRLLVNKIEMHPSIALVRPFNKGYDRMHRCKNDAQVEEVVSAGSLAYVYTPPAFGDEKVKSETKSEVKQEVKQEVRQDDVIQDGVPVKQEKAEVRAEDGVRIKRELSEEVQLPPPTNVKPEPHDDEAKVKLEDIPEKEAQPEDMEIYTTNHYIGLQLVEGAKSLDLSREVNDWKAMCMSNELYEEGLMFLSIQHLKNTALPDDVFEPGEVKPRPGKKVLKRGASEEPSKQQPPAKRQAHVQPRAPAAQQASSTATAAAG

SEQ ID NO: 8 - protein, 652 aa, synthetic construct
MAERTYGVTPPISTALPTEQEKALNKALHDELRAQGTFESRAETEKRKEVLAQLEKITNAFVQRAAREKHPKNAILIRDAIGRVFTYGSYRLGVYGPGSDMDTLVVAPKYVTVEQYFRIFPEVLVEMAPPAAITDLTPVPEAFVPIIKFEFSGISIDLIFCSIQTLIQLPADKSWSLADNNLLRGLSENAVRSLNGTRNTDEILHLVHEPATFKLALRAIKLWAQRKAIYANIMGYPGGAAWAVLVARVCQLYPKATSAVIVNKFFNIMLKWPWPLPVLLKDIEYNGPVTRVPVWNPKLYASDRNHKMPIITPAYPSTCATHNVGRSSMVVIQQELKKGAEVTEEIMLGRRPWKDLFTKHTFFTSGFKYYLTVISSSRTKKAQNVWSGFIESRVRLLVNKIEMHPSIALVRPFNKGYDRMHRCKNDAQVEEVVSAGSLAYVYTPPAFGDEKVKSETKSEVKQEVKQEVRQDDVIQDGVPVKQEKAEVRAEDGVRIKRELSEEVQLPPPTNVKPEPHDDEAKVKLEDIPEKEAQPEDMEIYTTNHYIGLQLVEGAKSLDLSREVNDWKAMCMSXELYEEGLMFLSIQHLKNTALPDDVFEPGEVKPRPGKKVLKRGASEEPSKQQPPAKRQAHVQPRAPAAQQASSTATAAAG

SEQ ID NO: 9 - protein, 652 aa, synthetic construct
MAERTYGVTPPISTALPTEQEKALNKALHDELRAQGTFESRAETEKRKEVLAQLEKITNAFVQRAAREKHGKNAILIRDAIGRVFTYGSYRLGVYGPGSDMDTLVVAPKYVTVEQYFRIFPEVLVEMAPPAAITDLTPVPEAFVPIIKFEFSGISIDLIFCSIQTLIQLPADKSWSLADNNLLRGLSENAVRSLNGTRNTDEILRLVHEPATFKLALRAIKLWAQRKAIYANIMGYPGGAAWAVLVARVCQLYPKATSAVIVNKFFNIMLKWPWPLPVLLKDIEYNGPVTRVPVWNPKLYASDRNHKMPIITPAYPSTCATHNVGRSSMVVIQQELKKGAEVTEEIMLGRRPWKDLFTKHTFFTSGFKYYLTVISSSRTKKAQNVWSGFIESRVRLLVNKIEMHPSIALVRPFNKGYDRMHRCKNDAQVEEVVSAGSLAYVYTPPAFGDEKVKSETKSEVKQEVKQEVRQDDVIQDGVPVKQEKAEVRAEDGVRIKRELSEEVQLPPPTNVKPEPHDDEAKVKLEDIPEKEAQPEDMEIYTTNHYIGLQLVEGAKSLDLSREVNDWKAMCMSNELYEEGLMFLSIQHLKNTALPDDVFEPGEVKPRPGKKVLKRGASEEPSKQQPPAKRQAHVQPRAPAAQQASSTATAAAG

SEQ ID NO: 10 - protein, 652 aa, synthetic construct
MAERTYGVTPPISTALPTEQEKALNKALHDELRAQGTFESRAETEKRKEVLAQLEKITNAFVQRAAREKHPKNAILIRDAIGRVFTYGSYRLGVYGPGSDMDTLVVAPKYVTVEQYFRIFPEVLVEMAPPAAITDLTPVPEAFVPIIKFEFSGISIDLIFCSIQTLIQLPADKSWSLADNNLLRGLSENAVRSLNGTRNTDEILRLVHEPATFKLALRAIKLWAQRKAIYANIMGYPGGAAWAVLVARVCQLYPKATSAVIVNKFFNIMLKWPWPLPVLLKDIEYNGPVTRVRVWNPKLYASDRNHKMPIITPAYPSTCATHNVGRSSMVVIQQELKKGAEVTEEIMLGRRPWKDLFTKHTFFTSGFKYYLTVISSSRTKKAQNVWSGFIESRVRLLVNKIEMHPSIALVRPFNKGYDRMHRCKNDAQVEEVVSAGSLAYVYTPPAFGDEKVKSETKSEVKQEVKQEVRQDDVIQDGVPVKQEKAEVRAEDGVRIKRELSEEVQLPPPTNVKPEPHDDEAKVKLEDIPEKEAQPEDMEIYTTNHYIGLQLVEGAKSLDLSREVNDWKAMCMSNKLYEEGLMFLSIQHLKNTALPDDVFEPGEVKPRPGKKVLKRGASEEPSKQQPPAKRQAHVQPRAPAAQQASSTATAAAG

SEQ ID NO: 11 - protein, 652 aa, synthetic construct
MAERTYGVTPPISTALPTEQEKALNKALHDELRAQGTFESRAETEKRKEVLAQLEKITNAFVQRAAREKHPKNAILIRDAIGRVFTYGSYRLGVYGPGSDMDTLVVAPKYVTVEQYFRIFPEVLVEMAPPGAITDLTPVPEAFVPIIKFEFSGISIDLIFCSIQTLIQLPADKSWSLADNNLLRGLSENAVRSLNGTRNTDEILRLVHEPATFKLALRAIKLWAQRKAIYANIMGYPGGAAWAVLVARVCQLYPKATSAVIVNKFFNIMLKWPWPLPVLLKDIEYNGPVTRVRVWNPKLYASDRNHKMPIITPAYPSTCATHNVGRSSMVVIQQELKKGAEVTEEIMLGRRPWKDLFTKHTFFTSGFKYYLTVISSSRTKKAQNVWSGFIESRVRLLVNKIEMHPSIALVRPFNKGYDRMHRCKNDAQVEEVVSAGSLAYVYTPPAFGDEKVKSETKSEVKQEVKQEVRQDDVIQDGVPVKQEKAEVRAEDGVRIKRELSEEVQLPPPTNVKPEPHDDEAKVKLEDIPEKEAQPEDMEIYTTNHYIGLQLVEGAKSLDLSREVNDWKAMCMSNELYEEGLMFLSIQHLKNTALPDDVFEPGEVKPRPGKKVLKRGASEEPSKQQPPAKRQAHVQPRAPAAQQASSTATAAAG

SEQ ID NO: 12 - protein, 652 aa, synthetic construct
MAERTYGVTPPISTALPTEQEKALNKALHDELRAQGTFESRAETEKRKEVLAQLEKITNAFVQRAAREKHPKNAILIRDAIGRVFTYGSYRLGVYGPGSDMDTLVVAPKYVTVEQYFRIFPEVLVEMAPPGAITDLTPVPEAFVPIIKFEFSGISIDLIFCSIQTLIQLPADKSWSLADNNLLRGLSENAVRSLNGTRNTDEILRLVHEPATFKLALRAIKLWAQRKAIYANIMGYPGGAAWAVLVARVCQLYPKATSAVIVNKFFNIMLKWPWPLPVLLKDIEYNGPVTRVRVWNPKLYASDRNHKMPIITPAYPSTCATHNVGRSSMVVIQKELEKGAEVTEEIMLGRRPWKDLFTKHTFFTSGFKYYLTVISSSRTKKAQNVWSGFIESRVRLLVNKIEMHPSIALVRPFNKGYDRMHRCKNDAQVEEVVSAGSLAYVYTPPAFGDEKVKSETKSEVKQEVKQEVRQDDVIQDGVPVKQEKAEVRAEDGVRIKRELSEEVQLPPPTNVKPEPHDDEAKVKLEDIPEKEAQPEDMEIYTTNHYIGLQLVEGAKSLDLSREVNDWKAMCMSNELYEEGLMFLSIQHLKNTALPDDVFEPGEVKPRPGKKVLKRGASEEPSKQQPPAKRQAHVQPRAPAAQQASSTATAAAG

SEQ ID NO: 13 - protein, 652 aa, synthetic construct
MAERTYGVTPPISTALPTEQEKALNKALHDELRAQGTFESRAETEKRKEVLAQLEKITNAFVQRAAREKHGKNAILIRDAIGRVFTYGSYRLGVYGPGSDMDTLVVAPKYVTVEQYFRIFPEVLVEMAPPAAITDLTPVPEAFVPIIKFEFSGISIDLIFCSIQTLIQLPADKSWSLADNNLLRGLSENAVRSLNGTRNTDEILKLVHEPATFKLALRAIKLWAQRKAIYANIMGYPGGAAWAVLVARVCQLYPKATSAVIVNKFFNIMLKWPWPLPVLLKDIEYNGPVTRVPVWNPKLYASDRNHKMPIITPAYPSTCTTHNVGRSSMVVIQQELKKGAEVTEEIMLGRRPWKDLFTKHTFFTSGFKYYLTVISSSRTKKAQNVWSGFIESRVRLLVNKIEMHPSIALVRPFNKGYDRMHRCKNDAQVEEVVSAGSLAYVYTPPAFGDEKVKSETKSEVKQEVKQEVRQDDVIQDGVPVKQEKAEVRAEDGVRIKRELSEEVQLPPPTNVKPEPHDDEAKVKLEDIPEKEAQPEDMEIYTTNHYIGLQLVEGAKSLDLSREVNDWKAMCMSNELYEEGLMFLSIQHLKNTALPDDVFEPGEVKPRPGKKVLKRGASEEPSKQQPPAKRQAHVQPRAPAAQQASSTATAAAG

SEQ ID NO: 14 - protein, 652 aa, synthetic construct
MAERTYGVTPPISTALPTEQEKALNKALHDELRAQGTFESRAETEKRKEVLAQLEKITNAFVQRAAREKHGKNAILIRDAIGRVFTYGSYRLGVYGPGSDMDTLVVAPKYVTVEQYFRIFPEVLVEMAPPAAITDLTPVPEAFVPIIKFEFSGISIDLIFCSIQTLIQLPADKSWSLADNNLLRGLSENAVRSLSGTRNTDEILKLVHEPATFKLALRAIKLWAQRKAIYANIMGYPGGAAWAVLVARVCQLYPKATSAVIVNKFFNIMLKWPWPLPVLLKDIEYNGPVTRVPVWNPKLYASDRNHKMPIITPAYPSTCATHNVGRSSMVVIQQELKKGAEVTEEIMLGRRPWKDLFTKHTFFTSGFKYYLTVISSSRTKKAQNVWSGFIESRVRLLVNKIEMHPSIALVRPFNKGYDRMHRCKNDAQVEEVVSAGSLAYVYTPPAFGDEKVKSETKSEVKQEVKQEVRQDDVIQDGVPVKQEKAEVRAEDGVRIKRELSEEVQLPPPTNVKPEPHDDEAKVKLEDIPEKEAQPEDMEIYTTNHYIGLQLVEGAKSLDLSREVNDWKAMCMSNELYEEGLMFLSIQHLKNTALPDDVFEPGEVKPRPGKKVLKRGASEEPSKQQPPAKRQAHVQPRAPAAQQASSTATAAAG

SEQ ID NO: 15 - protein, 652 aa, synthetic construct
MAERTYGVTPPISTALPTEQEKALNKALHDELRAQGTFESRAETEKRKEVLAQLEKITNAFVQRAAREKHGKNAILIRDAIGRVFTYGSYRLGVYGPGSDMDTLVVAPKYVTVEQYFRIFPEVLVEMAPPAAITDLTPVPEAFVPIIKFEFSGISIDLIFCSIQTLIQLPADKSWSLADNNLLRGLSENAVRSLNGTRNTDEILKLVHEPATFKLALRAIKLWAQRKAIYANIMGYPGGAAWAVLVARVCQLYPKATSAVIVNKFFNIMLKWPWPLPVLLKDIEYNGPVTRVPVWNPKLYASDRNHKMPIITPAYPSTCCTHNVGRSSMVVIQQELKKGAEVTEEIMLGRRPWKDLFTKHTFFTSGFKYYLTVISSSRTKKAQNVWSGFIESRVRLLVNKIEMHPSIALVRPFNKGYDRMHRCKNDAQVEEVVSAGSLAYVYTPPAFGDEKVKSETKSEVKQEVKQEVRQDDVIQDGVPVKQEKAEVRAEDGVRIKRELSEEVQLPPPTNVKPEPHDDEAKVKLEDIPEKEAQPEDMEIYTTNHYIGLQLVEGAKSLDLSREVNDWKAMCMSNELYEEGLMFLSIQHLKNTALPDDVFEPGEVKPRPGKKVLKRGASEEPSKQQPPAKRQAHVQPRAPAAQQASSTATAAAG

SEQ ID NO: 16 - protein, 652 aa, synthetic construct
MAERTYGVTPPISTALPTEQEKALNKALHDELRAQGTFESRAETEKRKEVLAQLEKITNAFVQRAAREKHPKNAILIRDAIGRVFTYGSYRLGVYGPGSDMDTLVVAPKYVTVEQYFRIFPEVLVEMAPPAAITDLTPVPEAYVPIIKFEFSGISIDLIFCSIQTLIQLPADKSWSLADNNLLRGLSENAVRSLNGTRNTDEILRLVHEPATFKLALRAIKLWAQRKAIYANIMGYPGGAAWAVLVARVCQLYPKATSAVIVNKFFNIMLKWPWPLPVLLKDIEYNGPVTRVRVWNPKLYASDRNHKMPIITPAYPSTCATHNVGRSSMVVIQQELKKGAEVTEEIMLGRRPWKDLFTKHTFFTSGFKYYLTVISSSRTKKAQNVWSGFIESRVRLLVNKIEMHPSIALVRPFNKGYDRMHRCKNDAQVEEVVSAGSLAYVYTPPAFGDEKVKSETKSEVKQEVKQEVRQDDVIQDGVPVKQEKAEVRAEDGVRIKRELSEEVQLPPPTNVKPEPHDDEAKVKLEDIPEKEAQPEDMEIYTTNHYIGLQLVEGAKSLDLSREVNDWKAMCMSNKLYEEGLMFLSIQHLKNTALPDDVFEPGEVKPRPGKKVLKRGASEEPSKQQPPAKRQAHVQPRAPAAQQASSTATAAAG

SEQ ID NO: 17 - protein, 652 aa, synthetic construct
MAERTYGVTPPISTALPTEQEKALNKALHDELRAQGTFESRAETEKRKEVLAQLEKITNAFVQRAAREKHPKNAILIRDAIGRVFTYGSYRLGVYGPGSDMDTLVVAPKYVTVEQYFRIFPEVLVEMAPPAAITDLTPVPEAFVPIIKFEFSGISIDLIFCSIQTLIQLPADKSWSLADNNLLRGLSENAVRSLNGTRNTDEILRLVHEPATFKLALRAIKLWAQRKAIYANIMGYPGGAAWAVLVARVCQLYPKATSAVIVNKFFNIMLKWPWPLPVLLKDIEYNGPVTRVRVWNPKLYASDRNHKMPIITPAYPSTCCTHNVGRSSMVVIQQELKKGAEVTEEIMLGRRPWKDLFTKHTFFTSGFKYYLTVISSSRTKKAQNVWSGFIESRVRLLVNKIEMHPSIALVRPFNKGYDRMHRCKNDAQVEEVVSAGSLAYVYTPPAFGDEKVKSETKSEVKQEVKQEVRQDDVIQDGVPVKQEKAEVRAEDGVRIKRELSEEVQLPPPTNVKPEPHDDEAKVKLEDIPEKEAQPEDMEIYTTNHYIGLQLVEGAKSLDLSREVNDWKAMCMSNKLYEEGLMFLSIQHLKNTALPDDVFEPGEVKPRPGKKVLKRGASEEPSKQQPPAKRQAHVQPRAPAAQQASSTATAAAG

SEQ ID NO: 18 - protein, 652 aa, synthetic construct
MAERTYGVTPPISTALPTEQEKALNKALHDELRAQGTFESRAETEKRKEVLAQLEKITNAFVQRAAREKHPKNAILIRDAIGRVFTYGSYRLGVYGPGSDMDTLVVAPKYVTVEQYFRIFPEVLVEMAPPAAITDLTPVPEAYVPIIKFEFSGISIDLIFCSIQTLIQLPADKSWSLADNNLLRGLSENAVRSLNGTRNTDEILRLVHEPATFKLALRAIKLWAQRKAIYANIMGYPGGAAWAVLVARVCQLYPKATSAVIVNKFFNIMLKWPWPLPVLLKDIEYNGPVTRVRVWNPKLYASDRNHKMPIITPAYPSTCCTHNVGRSSMVVIQQELKKGAEVTEEIMLGRRPWKDLFTKHTFFTSGFKYYLTVISSSRTKKAQNVWSGFIESRVRLLVNKIEMHPSIALVRPFNKGYDRMHRCKNDAQVEEVVSAGSLAYVYTPPAFGDEKVKSETKSEVKQEVKQEVRQDDVIQDGVPVKQEKAEVRAEDGVRIKRELSEEVQLPPPTNVKPEPHDDEAKVKLEDIPEKEAQPEDMEIYTTNHYIGLQLVEGAKSLDLSREVNDWKAMCMSNKLYEEGLMFLSIQHLKNTALPDDVFEPGEVKPRPGKKVLKRGASEEPSKQQPPAKRQAHVQPRAPAAQQASSTATAAAG

SEQ ID NO: 19 - protein, 652 aa, synthetic construct
MAERTYGVTPPISTALPTEQEKALNKALHDELRAQGTFESRAETEKRKEVLAQLEKITNAFVQRAAREKHPKNAILIRDAIGRVFTYGSYRLGVYGPGSDMDTLVVAPKYVTVEQYFRIFPEVLVEMAPPAAITDLTPVPEAYVPIIKFEFSGISIDLIFCSIQTLIQLPADKSWSLADNNLLRGLSENAVRSLSGTRNTDEILRLVHEPATFKLALRAIKLWAQRKAIYANIMGYPGGAAWAVLVARVCQLYPKATSAVIVNKFFNIMLKWPWPLPVLLKDIEYNGPVTRVRVWNPKLYASDRNHKMPIITPAYPSTCCTHNVGRSSMVVIQQELKKGAEVTEEIMLGRRPWKDLFTKHTFFTSGFKYYLTVISSSRTKKAQNVWSGFIESRVRLLVNKIEMHPSIALVRPFNKGYDRMHRCKNDAQVEEVVSAGSLAYVYTPPAFGDEKVKSETKSEVKQEVKQEVRQDDVIQDGVPVKQEKAEVRAEDGVRIKRELSEEVQLPPPTNVKPEPHDDEAKVKLEDIPEKEAQPEDMEIYTTNHYIGLQLVEGAKSLDLSREVNDWKAMCMSNKLYEEGLMFLSIQHLKNTALPDDVFEPGEVKPRPGKKVLKRGASEEPSKQQPPAKRQAHVQPRAPAAQQASSTATAAAG

SEQ ID NO: 20 - protein, 566 aa, synthetic construct
MAERTYGVTPPISTALPTEQEKALNKALHDELRAQGTFESRAETEKRKEVLAQLEKITNAFVQRAAREKHPKNAILIRDAIGRVFTYGSYRLGVYGPGSDMDTLVVAPKYVTVEQYFRIFPEVLVEMAPPAAITDLTPVPEAYVPIIKFEFSGISIDLIFCSIQTLIQLPADKSWSLADNNLLRGLSENAVRSLNGTRNTDEILRLVHEPATFKLALRAIKLWAQRKAIYANIMGYPGGAAWAVLVARVCQLYPKATSAVIVNKFFNIMLKWPWPLPVLLKDIEYNGPVTRVRVWNPKLYASDRNHKMPIITPAYPSTCCTHNVGRSSMVVIQQELKKGAEVTEEIMLGRRPWKDLFTKHTFFTSGFKYYLTVISSSRTKKAQNVWSGFIESRVRLLVNKIEMHPSIALVRPFNKGYDRMHRCKNDAQVEEVVSAGSLAYVYTPPADEEEKAKKEVKQEVKPEDLPPATNVKPEPHDDEAKVKLEDIPEKEAQPEDMEIYTTNHYIGLQLVEGAKSLDLSREVNDWKAMCMSNELYEEGLMFLSIQHLKNTALPDDVFEPGEVKPR

SEQ ID NO: 21 - protein, 524 aa, synthetic construct
MAERTYGVTPPISTALPTEQEKALNKALHDELRAQGTFESRAETEKRKEVLAQLEKITNAFVQRAAREKHPKNAILIRDAIGRVFTYGSYRLGVYGPGSDMDTLVVAPKYVTVEQYFRIFPEVLVEMAPPAAITDLTPVPEAYVPIIKFEFSGISIDLIFCSIQTLIQLPADKSWSLADNNLLRGLSENAVRSLNGTRNTDEILRLVHEPATFKLALRAIKLWAQRKAIYANIMGYPGGAAWAVLVARVCQLYPKATSAVIVNKFFNIMLKWPWPLPVLLKDIEYNGPVTRVRVWNPKLYASDRNHKMPIITPAYPSTCCTHNVGRSSMVVIQQELKKGAEVTEEIMLGRRPWKDLFTKHTFFTSGFKYYLTVISSSRTKKAQNVWSGFIESRVRLLVNKIEMHPSIALVRPFNKGYDRMHRCKNDAQVEEVVSAGSLAYVYTPPAGGGSGGGSMEIYTTNHYIGLQLVEGAKSLDLSREVNDWKAMCMSNELYEEGLMFLSIQHLKNTALPDDVFEPGEVKPR

SEQ ID NO: 22 - protein, 652 aa, synthetic construct
MAERTYGVTPPISTALPTEQEKALNKALHDELRAQGTFESRAETEKRKEVLAQLEKITNAFVQRAAREKHPKNAILIRDAIGRVFTHGSYRLGVYGPGSDMDTLVVAPKYVTVEQYFRIFPEVLVEMAPPAAITDLTPVPEAYVPIIKFEFSGISIDLIFCSIQTLIQLPADKSWSLADNNLLRGLSENAVRSLNGTRNTDEILRLVHEPATFKLALRAIKLWAQRKAIYANIMGYPGGAAWAVLVARVCQLYPKATSAVIVNKFFNIMLKWPWPLPVLLKDIEYNGPVTRVRVWNPKLYASDRNHKMPIITPAYPSTCCTHNVGRSSMVVIQQELKKGAEVTEEIMLGRRPWKDLFTKHTFFTSGFKYYLTVISSSRTKKAQNVWSGFIESRVRLLVNKIEMHPSIALVRPFNKGYDRMHRCKNDAQVEEVVSAGSLAYVYTPPAFGDEKVKSETKSEVKQEVKQEVRQDDVIQDGVPVKQEKAEVRAEDGVRIKRELSEEVQLPPPTNVKPEPHDDEAKVKLEDIPEKEAQPEDMEIYTTNHYIGLQLVEGAKSLDLSREVNDWKAMCMSNKLYEEGLMFLSIQHLKNTALPDDVFEPGEVKPRPGKKVLKRGASEEPSKQQPPAKRQAHVQPRAPAAQQASSTATAAAG

SEQ ID NO: 23 - protein, 652 aa, synthetic construct
MAERTYGVTPPISTALPTEQEKALNKALHDELRAQGTFESRAETEKRKEVLAQLEKITNAFVQRAAREKHPKNAILIRDAIGRVFTHGSYRLGVYGPGSDMDTLVVAPKYVTVEQYFRIFPEVLVEMAPPAAITDLTPVPEAYVPIIKFEFSGISIDLIFCSIQTLIQLPADKSWSLADNNLLRGLSENAVRSLNGTRNTDEILRLVHEPATFKLALRAIKLWAQRKAIYANIMGYPGGAAWAVLVARVCQLYPKATSAVIVNKFFNIMLKWPWPLPVLLKDIEYNGPVTRVRVWNPKLYASDRNHKMPIITPAYPSTCCTHNVGRSSMVVIQQELKKGAEVTEEIMLGRRPWKDLFTKHTFFTSGFKYYLTVISSSRTKKAQNVWSGFIESRVRLLVNKIEMHPSIALVRPFNKGYDRMHRCKNDAQVEEVVSAGSLAYVYTPPAFGDEKVKSETKSEVKQEVKQEVRQDDVIQDGVPVKQEKAEVRAEDGVRIKRELSEEVQLPPPTNVKPEPHDDEAKVKLEDIPEKEAQPEDMEIYTTNHYIGLQLVEGAKSLDLSREVNDWKAMCMSNELYEEGLMFLSIQHLKNTALPDDVFEPGEVKPRPGKKVLKRGASEEPSKQQPPAKRQAHVQPRAPAAQQASSTATAAAG

SEQ ID NO: 24 - protein, 652 aa, synthetic construct
MAERTYGVTPPISTALPTEQEKALNKALHDELRAQGTFESRAETEKRKEVLAQLEKITNAFVQRAAREKHPKNAILIRDAIGRVFTHGSYRLGVYGPGSDMDTLVVAPKYVTVEQYFRIFPEVLVEMAPPAAITDLTPVPEAYVPIIKFEFSGISIDLIFCSIQTLIQLPADKSWSLADNNLLRGLSENAVRSLNGTRNTDEILRLVHEPATFKLALRAIKLWAQRKAIYANIMGYPGGAAWAVLVARVCQLYPKATSAVIVNKFFNIMLKWPWPLPVLLKDIEYNGPVTRVRVWNPKLYASDRNHKMPIITPAYPSTCCTHNVGRSSMVVIQQELKKGAEVTEEIMLGRRPWKDLFTKHTFFTSGFKYYLTVISSSRTKKAQNVWSGFIESRVRLLVNKIEMHPSIALARPFNKGYDRMHRCKNDAQVEEVVSAGSLAYVYTPPAFGDEKVKSETKSEVKQEVKQEVRQDDVIQDGVPVKQEKAEVRAEDGVRIKRELSEEVQLPPPTNVKPEPHDDEAKVKLEDIPEKEAQPEDMEIYTTNHYIGLQLVEGAKSLDLSREVNDWKAMCMSNKLYEEGLMFLSIQHLKNTALPDDVFEPGEVKPRPGKKVLKRGASEEPSKQQPPAKRQAHVQPRAPAAQQASSTATAAAG

SEQ ID NO: 25 - protein, 652 aa, synthetic construct
MAERTYGVTPPISTALPTEQEKALNKALHDELRAQGTFESRAETEKRKEVLAQLEKITNAFVQRAAREKHPKNAILIRDAIGRVFTHGSYRLGVYGPGSDMDTLVVAPKYVTVEQYFRIFPEVLVEMAPPAAITDLTPVPEAYVPIIKFEFSGISIDLIFCSIQTLIQLPADKSWSLADNNLLRGLSENAVRSLNGTRNTDEILRLVHEPATFKLALRAIKLWAQRKAIYANIMGYPGGAAWAVLVARVCQLYPKATSAVIVNKFFNIMLKWPWPLPVLLKDIEYNGPVTRVRVWNPKLYASDRNHKMPIITPAYPSTCCTHNVGRSSMVVIQQELKKGAEVTEEIMLGRRPWKDLFTKHTFFTSGFKYYLTVISSSRTKKAQNVWSGFIESRVRLLVNKIEMHPSIALVRPFNKGYDRMHRCKNDAQVEEVVSAGSLAYVYTPPAFGDEKVKSETKSEVKQEVKQEVRQDDVIQDGVPVKQEKAEVRAEDGVRIKRELSEEVQLPPPTNVKPEPHDDEAKVKLEDIPEKEAQPEDMEIYTTNHYIGLQLVEGAKSLDLSREVNDWKAMCMSNKLYEEGLMFLSIQHLKNTALPDDVFEPGEVKPRPGKKVLKRGASEEPSKQQPPAKRQAHVQPRAPAAQQPSSTATAAAG

SEQ ID NO: 26 - protein, 652 aa, synthetic construct
MAERTYGVTPPISTALPTEQEKALNKALHDELRAQGTFESRAETEKRKEVLAQLEKITNAFVQRAAREKHPKNAILIRDAIGRVFTHGSYRLGVYGPGSDMDTLVVAPKYVTVEQYFRIFPEVLVEMAPPAAITDLTPVPEAYVPIIKFEFSGISIDLIFCSIQTLIQLPADKSWSLADNNLLRGLSENAVRSLNGTRNTDEILRLVPEPATFKLALRAIKLWAQRKAIYANIMGYPGGAAWAVLVARVCQLYPKATSAVIVNKFFNIMLKWPWPLPVLLKDIEYNGPVTRVRVWNPKLYASDRNHKMPIITPAYPSTCCTHNVGRSSMVVIQQELKKGAEVTEEIMLGRRPWKDLFTKHTFFTSGFKYYLTVISSSRTKKAQNVWSGFIESRVRLLVNKIEMHPSIALVRPFNKGYDRMHRCKNDAQVEEVVSAGSLAYVYTPPAFGDEKVKSETKSEVKQEVKQEVRQDDVIQDGVPVKQEKAEVRAEDGVRIKRELSEEVQLPPPTNVKPEPHDDEAKVKLEDIPEKEAQPEDMEIYTTNHYIGLQLVEGAKSLDLSREVNDWKAMCMSNKLYEEGLMFLSIQHLKNTALPDDVFEPGEVKPRPGKKVLKRGASEEPSKQQPPAKRQAHVQPRAPAAQQASSTATAAAG

SEQ ID NO: 27 - protein, 524 aa, synthetic construct
MAERTYGVTPPISTALPTEQEKALNKALHDELRAQGTFESRAETEKRKEVLAQLEKITNAFVQRAAREKHPKNAILIRDAIGRVFTYGSYRLGVYGPGSDMDTLVVAPKYVTVEQYFRIFPEVLVEMAPPAAITDLTPVPEAYVPIIKFEFSGISIDLIFCSIQTLIQLPADKSWSLADNNLLRGLSENAVRSLNGTRNTDEILRLVPEPATFKLALRAIKLWAQRKAIYANIMGYPGGAAWAVLVARVCQLYPKATSAVIVNKFFNIMLKWPWPLPVLLKDIEYNGPVTRVRVWNPKLYASDRNHKMPIITPAYPSTCCTHNVGRSSMVVIQQELKKGAEVTEEIMLGRRPWKDLFTKHTFFTSGFKYYLTVISSSRTKKAQNVWSGFIESRVRLLVNKIEMHPSIALVRPFNKGYDRMHRCKNDAQVEEVVSAGSLAYVYTPPAGGGSGGGSMEIYTTNHYIGLQLVEGAKSLDLSREVNDWKAMCMSNELYEEGLMFLSIQHLKNTALPDDVFEPGEVKPR

SEQ ID NO: 28 - protein, 524 aa, synthetic construct
MAERTYGVTPPISTALPTEQEKALNKALHDELRAQGTFESRAETEKRKEVLAQLEKITNAFVQRAAREKHPKNAILIRDAIGRVFTHGSYRLGVYGPGSDMDTLVVAPKYVTVEQYFRIFPEVLVEMAPPAAITDLTPVPEAYVPIIKFEFSGISIDLIFCSIQTLIQLPADKSWSLADNNLLRGLSENAVRSLNGTRNTDEILRLVPEPATFKLALRAIKLWAQRKAIYANIMGYPGGAAWAVLVARVCQLYPKATSAVIVNKFFNIMLKWPWPLPVLLKDIEYNGPVTRVRVWNPKLYASDRNHKMPIITPAYPSTCCTHNVGRSSMVVIQQELKKGAEVTEEIMLGRRPWKDLFTKHTFFTSGFKYYLTVISSSRTKKAQNVWSGFIESRVRLLVNKIEMHPSIALVRPFNKGYDRMHRCKNDAQVEEVVSAGSLAYVYTPPAGGGSGGGSMEIYTTNHYIGLQLVEGAKSLDLSREVNDWKAMCMSNELYEEGLMFLSIQHLKNTALPDDVFEPGEVKPR

SEQ ID NO: 29 - protein, 25 aa, synthetic construct
  REGION 1..25: note = Exemplary Tag
MASSHHHHHHSSGSENLYFQTGSSG

SEQ ID NO: 30 - protein, 6 aa, synthetic construct
  REGION 1..6: note = Exemplary Tag
HHHHHH

SEQ ID NO: 31 - protein, 8 aa, synthetic construct
GGGSGGGS

SEQ ID NO: 32 - protein, 28 aa, synthetic construct
  REGION 1..28: note = Flexible Loop
TNVKPEPHDDEAKVKLEDIPEKEAQPED

SEQ ID NO: 33 - protein, 90 aa, synthetic construct
  REGION 1..90: note = Flexible Loop
FGDEKVKSETKSEVKQEVKQEVRQDDVIQDGVPVKQEKAEVRAEDGVRIKRELSEEVQLPPPTNVKPEPHDDEAKVKLEDIPEKEAQPED

SEQ ID NO: 34 - DNA, 42 bp, synthetic construct
  source 1..42: mol_type = other DNA
  misc_feature 1..42: note = Initiator Nucleic Acid
  misc_feature 1: note = 5 prime Amino Modifier C12 linker (5AmMC12)
  misc_feature 38..42: note = Ribonucleoside ATP; note = RNA
  modified_base 21: mod_base = OTHER; note = IdeoxyI (DeoxyInosine)
  modified_base 32: mod_base = OTHER; note = iFluorT (Fluorescein dT)
  misc_feature 1..37: note = DNA
ttttttttttttttttttttnttttttttttntttttaaaaa

SEQ ID NO: 35 - RNA, 10 nt, synthetic construct
  source 1..10: mol_type = other RNA
  misc_feature 1..10: note = Sequence to be synthesised
ggcccagatg

SEQ ID NO: 36 - RNA, 10 nt, synthetic construct
  source 1..10: mol_type = other RNA
  misc_feature 1..10: note = Sequence to be synthesised
ttcttctcct

SEQ ID NO: 37 - RNA, 10 nt, synthetic construct
  source 1..10: mol_type = other RNA
  misc_feature 1..10: note = Sequence to be synthesised
ggccaaaacc

SEQ ID NO: 38 - RNA, 10 nt, synthetic construct
  source 1..10: mol_type = other RNA
  misc_feature 1..10: note = Sequence to be synthesised
gcctgttcag

SEQ ID NO: 39 - RNA, 10 nt, synthetic construct
  source 1..10: mol_type = other RNA
  misc_feature 1..10: note = Sequence to be synthesised
atccgtagcc

SEQ ID NO: 40 - RNA, 10 nt, synthetic construct
  source 1..10: mol_type = other RNA
  misc_feature 1..10: note = Sequence to be synthesised
ggggctgtac

SEQ ID NO: 41 - RNA, 10 nt, synthetic construct
  source 1..10: mol_type = other RNA
  misc_feature 1..10: note = Sequence to be synthesised
cagcgcgcac

SEQ ID NO: 42 - RNA, 10 nt, synthetic construct
  source 1..10: mol_type = other RNA
  misc_feature 1..10: note = Sequence to be synthesised
aggtcctcac

SEQ ID NO: 43 - RNA, 10 nt, synthetic construct
  source 1..10: mol_type = other RNA
  misc_feature 1..10: note = Sequence to be synthesised
cccaggcgca

SEQ ID NO: 44 - RNA, 10 nt, synthetic construct
  source 1..10: mol_type = other RNA
  misc_feature 1..10: note = Sequence to be synthesised
acgtgctgcc

SEQ ID NO: 45 - RNA, 10 nt, synthetic construct
  source 1..10: mol_type = other RNA
  misc_feature 1..10: note = Sequence to be synthesised
ccgcatcagc

SEQ ID NO: 46 - RNA, 10 nt, synthetic construct
  source 1..10: mol_type = other RNA
  misc_feature 1..10: note = Sequence to be synthesised
cgccgcatca

SEQ ID NO: 47 - RNA, 10 nt, synthetic construct
  source 1..10: mol_type = other RNA
  misc_feature 1..10: note = Sequence to be synthesised
tgtaacacca

SEQ ID NO: 48 - RNA, 10 nt, synthetic construct
  source 1..10: mol_type = other RNA
  misc_feature 1..10: note = Sequence to be synthesised
cgggccccaa

SEQ ID NO: 49 - RNA, 10 nt, synthetic construct
  source 1..10: mol_type = other RNA
  misc_feature 1..10: note = Sequence to be synthesised
agggtcccca

SEQ ID NO: 50 - RNA, 10 nt, synthetic construct
  source 1..10: mol_type = other RNA
  misc_feature 1..10: note = Sequence to be synthesised
gggtgggaga

SEQ ID NO: 51 - RNA, 10 nt, synthetic construct
  source 1..10: mol_type = other RNA
  misc_feature 1..10: note = Sequence to be synthesised
tcttccccag

SEQ ID NO: 52 - RNA, 10 nt, synthetic construct
  source 1..10: mol_type = other RNA
  misc_feature 1..10: note = Sequence to be synthesised
ggcctgtgat

SEQ ID NO: 53 - RNA, 10 nt, synthetic construct
  source 1..10: mol_type = other RNA
  misc_feature 1..10: note = Sequence to be synthesised
gggcctacca

SEQ ID NO: 54 - RNA, 10 nt, synthetic construct
  source 1..10: mol_type = other RNA
  misc_feature 1..10: note = Sequence to be synthesised
gatgcctgga

SEQ ID NO: 55 - RNA, 10 nt, synthetic construct
  source 1..10: mol_type = other RNA
  misc_feature 1..10: note = Sequence to be synthesised
tagaaatagc

SEQ ID NO: 56 - RNA, 10 nt, synthetic construct
  source 1..10: mol_type = other RNA
  misc_feature 1..10: note = Sequence to be synthesised
cccagatgcg

SEQ ID NO: 57 - RNA, 10 nt, synthetic construct
  source 1..10: mol_type = other RNA
  misc_feature 1..10: note = Sequence to be synthesised
ggttttggcc

SEQ ID NO: 58 - RNA, 10 nt, synthetic construct
  source 1..10: mol_type = other RNA
  misc_feature 1..10: note = Sequence to be synthesised
ccaccaaact

SEQ ID NO: 59-64 - skipped (6 entries)

SEQ ID NO: 65 - RNA, 20 nt, synthetic construct
  source 1..20: mol_type = other RNA
  misc_feature 1..20: note = Sequence to be synthesised
gattgcgggcgatgggtgag

SEQ ID NO: 66 - RNA, 20 nt, synthetic construct
  source 1..20: mol_type = other RNA
  misc_feature 1..20: note = Sequence to be synthesised
aagcagatcaaatgtgtaga

SEQ ID NO: 67 - RNA, 20 nt, synthetic construct
  source 1..20: mol_type = other RNA
  misc_feature 1..20: note = Sequence to be synthesised
atcaatcctagtacatccgc

SEQ ID NO: 68 - RNA, 20 nt, synthetic construct
  source 1..20: mol_type = other RNA
  misc_feature 1..20: note = Sequence to be synthesised
gcatgaacctttcgacatct

SEQ ID NO: 69 - RNA, 20 nt, synthetic construct
  source 1..20: mol_type = other RNA
  misc_feature 1..20: note = Sequence to be synthesised
acactgacagtgctcaataa

SEQ ID NO: 70 - RNA, 20 nt, synthetic construct
  source 1..20: mol_type = other RNA
  misc_feature 1..20: note = Sequence to be synthesised
aagaatcagtaaagtatggg

SEQ ID NO: 71 - RNA, 20 nt, synthetic construct
  source 1..20: mol_type = other RNA
  misc_feature 1..20: note = Sequence to be synthesised
ccgactgataaccgcgacgg

SEQ ID NO: 72 - RNA, 20 nt, synthetic construct
  source 1..20: mol_type = other RNA
  misc_feature 1..20: note = Sequence to be synthesised
agttactatatcatcagcgg

SEQ ID NO: 73 - RNA, 20 nt, synthetic construct
  source 1..20: mol_type = other RNA
  misc_feature 1..20: note = Sequence to be synthesised
acacaggtattccagcgagg

SEQ ID NO: 74 - RNA, 20 nt, synthetic construct
  source 1..20: mol_type = other RNA
  misc_feature 1..20: note = Sequence to be synthesised
tttttttttttttttttttt

SEQ ID NO: 75 - RNA, 40 nt, synthetic construct
  source 1..40: mol_type = other RNA
  misc_feature 1..40: note = Sequence to be synthesised
tttttttttttttttttttttttttttttttttttttttt

SEQ ID NO: 76 - protein, 675 aa, Chaetomium thermophilum
  source 1..675: note = var. thermophilum DSM 1495
MATERQWGVTPPISTALPTENEKRLHDALHKELRAQGTFESPAERAKREKVLAELEKITNVFVKRAAQEKQPKNSILIRDAIGRVFTYGSYRLGVYGPGSDIDTLVVAPKYVTVEQYFRIFPQVLVEMAPPGAIKDLTPVPEAFVPIIKFEFDGISIDLIFCSIQTLMQLPADPNWSLADNNLLRGLSENEVRSLNGTRVTDEILNLVPEPATFKLALRAIKLWAQRKAIYANIMGFPGGVAWAMLVARVCQLYPKATSAVIVNKFFHIMVKWPWPLPVLLKDIEYGCPVTRVAVWNPKLYASDRNHKMPIITPAYPSMCATHNVGNSTMAVIQQELEKGAQITDEIMVGRRPWKDLFTKHTFFTNGFKYYLTVISSSRTKQAQNAWSGFIESRVRLLVNKLEMHPDIVLARPFNKGYDRVHRVKDDRQLEEVVGQGSLAYLYKAANEADKVKGMSKPEPKQDATMKQEIKTEVKTEDDVTVKQEKQENGEPMPPPAPTIKFEPNDSKTNMNNIPKHGQSPAVDHTNGNVKLENSENVKLEDIPVKEEEEESFMEIYTTNHYIGLQLVEGAKQLNLSREVNDWKAMCMSNEIYKAETMFLAIQHLKNTALPDDVFEPGEKKPQPAKKSRKRVASEEPSRQGSAPPAKRQAQGNEGVQSGQLQQPQGNQGKTLART

SEQ ID NO: 77 - protein, 653 aa, Thermothelomyces thermophilus
  source 1..653: note = ATCC 42464
MAERIYGVTPPISTALPTEEEKRLNNALHQELRAQGTFESPAETEKRKEVLRQLEKITNVFVQRAAAEKEPKNTILIRDAIGRVFTYGSYRLGVYGPGSDMDTLVVAPKYVTVEQYFRIFPEVLVEMAPPGAITDLTPVPEAFVPIIKFEFSGISIDLIFCSIQTLKQLPADKNWSLADNNLLRGLSENEVRSLNGTRVTDDILNLVPEPATFKLALRAIKLWAQRKAIYANIMGYPGGVAWAMLVARVCQLYPKATSAVIVNKFFHIMLKWPWPLPVLLKDIEYGCPVTRVAVWNPKIYASDRNHRMPIITPSYPSMCATHNVGRSSMAVIKDELEKGVQVTEDIMRGKRPWKDLFTKHTFFTSGFRYYLTVISSSRTKKAQNVWSGFVESRVRLLVNKLEMHPSISLARPFNKGYDREHRCKNNAQLEEVVSLGSLAYMYKPATGEAKAEAKKEVKTEVKHESGASVKNEAPEAKGEDGVRAKRENGDEAQLPPATGIKPEPTDGAGADVKLEDIPVKKDEPEEMTIYTTNHYIGLQLVEGAKTLDLSREVNDWKAMCTSNELYEEGTMFLSIQHVRNTALPDDVFEPGETRPRPAKKSLKRVASEDPGNKQTQPPAKKQVQDKAPPAAQQQQQQQHQQQQQPASTAAAAA

SEQ ID NO: 78 - protein, 652 aa, Thermothielavioides terrestris
  source 1..652: note = NRRL 8126
MTERTYGVTPPISTALPTEQEKALNKALHDELRAQGTFESRAETEKRKEVLAQLEKITNAFVQRAAREKHGKNAILIRDAIGRVFTYGSYRLGVYGPGSDMDTLVVAPKYVTVEQYFRIFPEVLVEMAPPAAITDLTPVPEAFVPIIKFEFSGISIDLIFCSIQTLIQLPADKSWSLADNNLLRGLSENAVRSLNGTRVTDEILHLVPEPATFKLALRAIKLWAQRKAIYANIMGYPGGVAWAMLVARVCQLYPKATSAVIVNKFFNIMLKWPWPLPVLLKDIEYNGPVTRVPVWNPKLYASDRNHKMPIITPAYPSMCATHNVGRSSMVVIQQELKKGAEVTEEIMLGRRPWKDLFTKHTFFTSGFKYYLTVISSSRTKKAQNVWSGFIESRVRLLVNKIEMHPSIALARPFNKGYDRMHRCKNDAQVEEVVSAGSLAYVYTPPAFGDEKVKSETKSEVKQEVKQEVRQDDVIQDGVPVKQEKAEVRAEDGVRIKRELSEEVQLPPPTNVKPEPHDDEAKVKLEDIPEKEAQPEDMEIYTTNHYIGLQLVEGAKSLDLSREVNDWKAMCMSNELYEEGLMFLSIQHLKNTALPDDVFEPGEVKPRPGKKVLKRGASEEPSKQQPPAKRQAHVQPRAPAAQQPSSTATAAAG

SEQ ID NO: 79 - protein, 566 aa, Pyronema omphalodes
  source 1..566: note = strain CBS 100304
MSAPPKQYGTTPPINTNPPTKEDMERNSELVQELKNQNCFEEKAESDKRVKVLEILQTLGEAFVKKACAAKGLPDHLVNNSGGKIFTFGSYRLGAYGPGSDIDTLLVVPAHIERADFFQHVPKMLSELNPPAEEVSPVPDAFVPIIKFELQSISIDLIFAKVPSVNSIPRDMTLENKDILKGCDEANLRGLNGVRLTDELLGLVPHPVNFRMALRAIKLWAKSRAIYANVMGFPGGAAWAMLVAKICQLYPMAVSAVIVCKFFTIYTKWKWPQPVLLKDILDEGGQGAGLRVWNPKIYASDRGHLMPVITPNYPCTCSTHNITKSTKAVILREMDRSLNIIHEIMDGKSPWSKLFAPHTFFTQDYRYYLRVISAARTKDEQLLWSGLVESKLRHLVSKLEMLDNIQLAHPFNKGFEYEVECNNEDEVMRVIRGENIDQSEANGTAVDSNGKSSKVKAYTTSFYVGLVLDTSKSKQFDISWACHEFYDICKTWNLYNDDMHSIHVVNTRNFELPDNVFKPGETKPTKKTKTIKRKIPNAAGKKRGADEISAPDSKRAHINEVPPNAN

SEQ ID NO: 80 - protein, 598 aa, Tilletia indica
MTKHLGVTPIVSDASPTAHEIALETQLVTELTTQNCFESPQDSRLREVVLGKADKLVKEFVYRAAIARGLPESAARELGGKIFTFGSYRLGVHGPGSDIDTLCVVPKHVQREDFFTIFEQMLRQREDVTDVAPVPEAYVPLIAVNFMGIAIDFLFARLSLSRIDDSLDLSDNTILKNMDDQDIRSVGGSRVTDEILRLVPNVETFHTALRAIKLWAQRRAIYKNMVGFPGGAAWAMLVARICQLYPNGNAAVIIERFFIIMFQWNWPQPVMLKHHIYEADPILKVWNPRLYAADRSHRMPIITPAYPGTCATHNITASTQAVLTLEFKRGIEIMSAMSASITNPALPQTTWATLFESRRFFEEYKYYLQIIASSGSADLQLKWAGTVESKLRHLVLSTEAQPGVKIAHPYTREIDLVSECHTDEEVRRVATGDVPPEVAARARTVEGEGVLPESKEVDKLKEIESKEEEARKKGRRTIWTTTFYIGLALHDGSDDKTGQSAPKRLDLVGPIQDFKGRCTEWATYDESTMGVVVRHVKRSQLPDSVFPDGKRPAAPVKKVVKKRKRNAGMAEAAAETKEEEGADEQEAKRARTNGDSAA

SEQ ID NO: 81 - protein, 574 aa, Clathrospora elynae
MNGPPKTQYGVTSAISEAAPTEEDRLLNDKLIETLKRENVFETPEGNAKREEVILHLQKVVEEFVRRVGKQKGVPQSTIDAAGGKVFTFGSYALGVHGPSSDIDTLVVAPKFVTIDEFFQTFPPTFKEMSRVEDIKEFVPVEDAFVPIIKMEYRGVSIDLLFASLPRMASIPKDMDTIDKKNLEGLGESATRSVNGTRVTKELLAAVPQNNSFRHALRAIKLWSSRRGIYGAVFGYPGGAAWAIMVARICQLYPFANGATIVSKFFSLMYKWTWPRPVMLKHIEEGAMGLRVWNPQIYGGDRAHLMPIITPAFPSTCATHTVMPSTLRIMKEEFGRADKILQHVFAGTKKWDALFERHSFFTKDHKYYLSVVAASRTKEANSTFSGLVQSKIRHIVKGIDDGQTGVDIARPYIDYFERYHRCKDEDQIFQVCQGKLDHMIPASELPAEGTAPANGDSHIMYTTTFYIGLTLPLGTATEGTKSLDISYPVSQFKNFITDADKFDKETMSVKVVHTRSSALPDDVFVQGETRPKKPVKEKKKKEAKSAKRQFADTGLDVRDSRLTKPSQRSSASRE

SEQ ID NO: 82 - protein, 545 aa, Drechslerella brochopaga
MPASVDVLKRLQKITEEFVRQVYTSKNQNELTTNSAGGKVFTYGSYRLGVVGPGSDIDTLVVAPKLVTREDFFKFYPPLLKALNKANEPPVIEELAPVPDAFVPIIKFVMSGISIDLIFCRLGVAQVPADMTLEDKNLLRGLDEKELRSLNGTRVTDEILTLVPVPAVFKHALRAIKIWAKCRAIYGNVYGFPGGAAWAMLVARICQLYPSAVSAVIVSKFFRILGQWNWPQPVLLKPIEDGPLNVRVWNPKLYPSDRNHLMPIITPAYPSTCSTHNITPSTKAVILGEMAKAADIVDRIITGNGNWNQLFQRHTFFTKDYKYYLTVKASARDQEQSVKWSGLVESKIRHLVMKLEMLSDVIASARPYVKPFEKVQYCRNQEEAHKIAMGQHVPDSPPAEAANAATGETAGEVSEQEKEGGVPVWITTFHIGIELTPGMVGQVNISWPAQEFYIMCHNWEGYEENMHCVKLELLRNWNLPDECFDFSKGDAKPSRPIVKRKVIRKVVSSSEVSQSGMKRTHAEENPNADEGDVKRTKVNAPVTVQ

SEQ ID NO: 83 - protein, 631 aa, Magnaporthiopsis poae
  source 1..631: note = ATCC 64411
MAERQLGVTPPVSVSLPSEFELQATDALLAELRAQNSFESPLETQKRHRVLASLQTIADAFVKVVAQEREPHNAVLIKDARAKVFAYGSLRLGVWGPGSDIDTLVVGPRYVTREDYFKHFPDLLVKMAPPGAITDLAVVQEAFVPIIKFEYHGISIDLIFSRIATLKQLPSDPAWDLKDNNLLRGLDEKELRSVNGTRVTDEILSLVPEQNIFRTALRAIKLWAQRRAVYGNIMGFPGGAAWAMLVARVCQLYPKATSSVIVNKFYSILLQWPWPQPVLLKPIGDGPLQVRVWNPRLYKGDSYHLMPVITPAYPSMCATFNVTRSSMTVIYRELQLAAEVTNNIMCSTKPWKDLFTKHTFFTKDFKYYMQVISASRDKEAHKIWSGFVESKVRMLVQSLERHDSIAVARPFIKGFERFHRYRNEEQFAQILDGNLQHMVKAGGDASPSQDGIQAKKEDNSSVKAEEDAKVKQENGETPVKQENGVKVKGEESEEAPLASIPEHSPVDTKGDSKAGDEALEMYTTTHYIGLELNPAAKSLDLSFQVNDFRALCHNWEKYKDELSDRCYVNIKDLRNFALPEDLFEPGEVKPVKSVKKRPAAGANATGDKKRPANEDNSNPAKRRQAASVAAA

SEQ ID NO: 84 - protein, 631 aa, Cryptococcus depauperatus
  source 1..631: note = CBS 7841
MLGVTPPISIEAPKPGDIHSSEALMTDLIAMNQFESDSERKIREKLLSNIAQLVVKFVHDVSLKIGMSEKMASESGGRIYTSGSYRLGVHGPGSDIDTICVCPRHIYREHFFGEFQEMLRAWPEVTEITAVTGAFVPVMKTVISGVEVDLLFARVNLPEAGDSLDIEKDEILRGVDDASQRSLNGPRVTDMILNLVPDVTTFRTALRSIRLWARRRGIYSNVLGFPGGAAWALLTARICQLYPNATPSIIVGKFFPIYYQWNWPQPVILKKIENGPPNMQHAVWNPKLDRRDMAHRMPVITPAYPSTCSTHNITASTMSIISKEMLRAMQITDDILREPGASWAPLFEKVDFFSMYKTYVQVVASASTAEGIKDWSGTVEARIRTLVQDLENTDSILTAHPLVGGVSRVFYCTTEEEQAAASQGELTSEMIDRTEEEVTDKEHRNIFTKSFFIGLEIEKKTKEGGGRVLNLFYPSKKFCSMCQSWDRYNEMEMSVILRPAKRSDLPSFVFPDGIPISKKKAKRQQQNGSADASLSDSVEGQGPSKRTKAAPPSPNQASNPRCNGLPVQNGSDPVSSKIGEEVDIKPPPGIENLPPLSNAAMSSFATAAKGVATTQDFNKEGLVVLNQQSTL

SEQ ID NO: 85 - protein, 570 aa, Golovinomyces cichoracearum
MAEKSFGVTAPLSVTPPTESENQASSALIEELKRQNNYENAIETANRQKVLNSLQLITEEFVRQVSRAQGLPANLIKSAGGMVVTFGSYKLGVIGPGSDIDTLIVAPQNVTKEDFFSRFPDLLRSMATEGNITELTAKPDAFAPCITLKYAGIDIDLLFGRVKLSQVPRNLSLLNQNMLRGLNNEEVRSLNGVRVADEILNLVPEPAIFRTALRTIKLWGTRRAIAGNIYGFPGGATWAILVARICQLYPKATSSTIVFKFFRIMEKWRWPMPVLLKEIDNLNALGLKVWNPKIYGSDKNHIMPIITPAYPETCTTHNFSLSTKAILEKELKRGGDITDRVMSGKASWKDLFAKHTFFTNGYKYYLSVVSASRNKESQLFWSGFVESKVRLLVNKLEYHPSIALAHPFNKGFTRVHRCRTEEEVDLVKNGSLKFHATDIATSTTGHGLSVETTSKEKLNETKNVEQDENLIMVYTATHYIGLELAKGAKSLDLSYEVEEFKSICTSSEAYNQEENSLGVAHTKNCDLPDDVFTEGELKPTRPLKQKKKRPATEDSNPVPSKRQQTSLIAV

SEQ ID NO: 86 - protein, 599 aa, Hortaea werneckii
MAEKQYGVTPAFSLEPPSPKDLKLNDALLAEFKAQNNFAPQSDTEKREAILKKLEGLLQRMVQEVGRKKGLPQSILEVAGGKVFTYGSYRLGVYGPNSDVDTLMVGPKHVTREDFFEHMPPLIRSAWAEDQIGGLVPVPGIGTPIIKLELEGVDIDLIYSSLQLSSIPKDIELKDDNLLRGLDDTDRRCVNGTRVTNRILELVPQTKTFRLALRAIKLWSSQRAIYGNIVGFPGGAAWAILVARVCQLYPKAAAPLLISKFFFIMKRWNWPKPVFLQHKEETSLQLREWDPIQYRGDGFHLMPILTPAYPSTNTAHTVGPSTKMIIMRELERGENIVNDIYANKRPWKDLFQRHTFFTNAYKHYICVVVAAKNKDAHDNWSGLVNSKLKFLVKGIEDSGGSSVELVQPFNKGFSRVHECQTNEQVEKVLDGSLECQVKETKTTEEGNDTAIANGVAQTDTEGLEVPKADSEARAEGSGTTKLWTTTFYLGIGLKKGATDLDISVPVRNLQGDCTAWADYNPDLHSIKIKHIRNFNLPDDVFKEGEVKPQRPKKKTNAAKAAEATSNKRSFSDTGLDPNADPAKRRQSGNVPTPVANGSV

SEQ ID NO: 87 - protein, 588 aa, Valsa sordida
MAEIQYGMTPPLSTELPKDPEKRANDALFAELKAQNTYEHATETQKREKVLKSLQSIADVFVQKVAQRVHKESPAIQKSARGRVFTYGSYRLGVFGPGSDIDTLVVAPKYVTREDFFELFPGLLKELAPAGSITDLTAVTDAFVPIIKFEYSDISIDLIFSRIATLTEIPALTTKWDLLDNNLLRGLDDAELRSLNGTRVTDDILKLVPEQTSFRLALRAIKLWAQRRAIYANIMGFPGGAAWAMLVARICQLYPKANGAVIVDKFFHIIRRWPWPQPVLLKNPEDGPLQVRVWNPKVYKGDSYHLMPVITPAYPSTCSTFNVTHSNKAVIQKELDHFADVVQQIMMGKLPWKSLFVKHTFFTKDYKYYIAVIAASTTKENSKIWGGFVESKIRLLVQGLERHSSIRLARPFNKGYERKHRTVSGMNEWHDILDGSLDYVVKDEQVADEKAKTEPGNDAVKTEPTSPAKVKQEEGIESLEIKDDAKETDGKNEQDEEDAEVKPETEDPDSPKDVYTSTHYIGIELHDEHYSWSLPDDVFEAGETKPTRPLKKKAAQPPNGAKRKTPTEGALNSAIDAAKRQKTSTVSA

SEQ ID NO: 88 - protein, 588 aa, Wallemia mellicola
MAEKQWGITPPISLASPTDLEKQVDAGLIQELKDQNTIESEQESLNREKVLALVQSLVKEFVKRVSIRNGMSESLADQAGGKIFTFGSYRLGVNQPGADIDTLCVVPKHVSREDFFDVFEPLLKTREEVSVCAGVPDAYVPIIKTTISGIEIDFLVARLALATIPDDLELADDNLLKNLDERCVRSLNGSRVTDEILRVVPNVQVFRESLRCIKLWAQRRAIYSNVNGFLGGAAWAMLVARICQLYPNENAGAVIAKFFTILYQWKWPHPVLLKEIEDGPLKVRVWNPKVGSSTLLIYPSDRAHRMPIITPAYPSTCSTHNVGPSTQEVMRQEFKRGMDILDGIHKGTTTWSQLFNKHEFFSKYKHYLQIIASGQTAESQKKWSGAVESKVRQLVSKLELVDGIELAHPFVKGFSEVFICLNDEEVQHAAEYNPSEEVKERSKKPEEYENKEAEDGIHRVYISTFYIGLAIQPRNPETNEKRTLNLTYPTNEFMKLTKLWDQFVEGEMKIFVKNIKASALPDVVFEGGIRTSSSKSSKKKSKRPSNVMDESNGENEQNKRLKPDNTSNNEQKSEATALAPSAVPTEST

SEQ ID NO: 89 - protein, 586 aa, Xylaria flabelliformis
MAEKTWGITKPISSAPPTPAESHATALLLEELRRQNTFETASEIKKREKVVNDLQRIADEFVRKVARAKEPHNEVLIRDARGEVFTYGSYCLGVYGPGSDIDTLVTAPRYVTRDDYFKYFPDLLTEMAPPGAITNLTAVEDAFVPIIKFEYWGISIDLIFSRIATLTQFPPHKQLELTSNEHLRGLDDRELRSLNGTRVTKEILNLVPEQSTFRTALRAIKLWAQRRAIYANIIGFPGGAVWAMMVARVCQLYPKATSATIVGKFFLVMKGWPWPIPVQLKHMEDGPLNVRVWNPKIYKSDSFHLMPVITPAYPQTCATFNITKSTKTIIQRELERGVELTDKILGSERPWKDLFVKHTFFTQDHKYYLAVIATSTTKEAHKIWSGFVESKVRILVGELERHSSIALARPFNKGFERQHLTKTDQQAAEVQSGSLAYVTEDEDNSDDATVTKAENGTDGNADSSDQKVTAENTTQPRRTYTTTHYIGLELAEGSKSLDLSYQVNAFKQLCSEWEKYSAEFNSLSVQHVKNINLPEDVFEPGETKPARPLKKPTANGTAPTTQRKRNAPTEGHPPPAKRQQSSAAAA

SEQ ID NO: 90 - protein, 591 aa, Chaetomium globosum
  source 1..591: note = CBS 148.51
MSEIIYGVTPPISTTLPTEPEKRLNHALHQELRAQGTFESPQETEKRKEVLRQLEKITTVFVQRAAAQKEPKNTFMIRDAIGRVFTYGSYRLGVYGPGSDMDTLVVAPKYVTVEQYFEIFPEVLVEMAPPGAITDLTPVPEAFVPIIKFEFSGISIDLIFCSIQTLKQLPDEKNWSLADNNLLRGLSENEVRSLNGTRVTDDILHLVPEPATFKLALRAIKLWAQRKAIYANIMGYPGGAAWAMLVARVCQLYPKATSAVVVNKFFHIMFKWPWPLPVLLKDIEYGSPVTRVPVWNPKSSRRAGPKRHRTSGRASSNRGCGCWLTDLRGTILLPWPGPSTRVTTENTDARTIGSWEEVVSVGSLAYVYKPPADSEEKAKAEPKVEPKREVKQEIKAENREPAAAEEKLETRGEDGMRIKQEHSESTQPPPPSNVKPEPHHDTNSEVKLEDIPQKKKDEPDEMEIYTTNHYIGLQLVEGAKSLDLSREVNDWKSMCTSNELFDEGLMFLSIQHLKNTNLPDDVFEPGETKPRPVKKNLKRAASEGPGNMQPQPPVKRHAPGKPSSHSAQQQQQQQQKQRQQRPASTAAAAVA

SEQ ID NO: 91 - protein, 589 aa, Lachancea thermotolerans
  source 1..589: note = CBS 6340
MNQQKTYGVTGPISTAGPTAAENQLNDALIQELKKEKSFESEEDTKKRVEVLRILQNLAQEFVYQVSKKRNMSDGMAKDAGGKIFTYGSYRLGVHGPGSDIDTLVVVPKHVTREDFFTVFDQILRTRSELEEIAPVPDAFVPIIKIKFSGISIDLICARLDIAQVPVNLTLADKNLLRNLDEKDLRALNGTRVTDEILQLVPKPTSFKIALRAIKLWAQRRAVYANIFGFPGGAAWAMLVARICQLYPNACSAVILTRFFHILTKWNWPQPVLLKPIEDGPLQVRVWNPRIYAQDRSHKMPVITPAYPSTCATHNISESTKKVILAELERGAQISSEIFSNKKTWSDLFQKHDFFYKYKFYLTVMASTSGSSEQHLKWSGLVESKLRLLVQKLETLGGINLAHPFTKPFEASYVYDNEAQCKDIIDNYGTHKAQDILAQYTEVTDDNKDTDGVKDKAQVHITTMYIGLDVALDGKKQVDIHVPCSDFFNLCRSFSEYDDTDMFSLMIKYVKLYDLPDNVYVEGEERPVKHSKRKKLGKDSKKSKRPKSNSLKAEEPTSGTTNSEKNNSQGPNKPVKHEPPMQTSPNPLA

SEQ ID NO: 92 - protein, 558 aa, Schizosaccharomyces octosporus
  source 1..558: note = yFS286
MSVKQWGITPPISTAPATEQENALNTALIDELKRQNLFESSAESERRIKVLDDLQRIATEFVKKVSLAKHMNEKMANEAGGKIFTYGSYRLGVYGPGSDIDTLLVVPKHVSRENFFQDLEPMLREREEITELASVPDAYVPIIKFKYFDISIDLIFARLSVPKVPRTLELSDNNLLKGVEERCILSLNGTRVTDQILQLVPNRAVFKHALRAVKFWAQRRAIYANVIGFPGGAAWAMMVARICQLYPNAVSSVIVAKFFRILHQWNWPQPILLKPIEDGPLQVRIWNPKLYPSDKAHRMPIITPAYPSTCATHNITPSTQAIILSEMVRAGEIADQIMVGSVGWSALFQKHDFFHRYKHYLMITAASKTAEGQLKWSGLVESKLRHLVTRLELVEAIALAHPFNKGFDKIHNCKSEEEAHQLASGISLDVASQTADLERLAADANGQNENEDEKEEKKIYPVYTTTFYIGLELEKKKGQPIKKLDISWPAQEFYELCKKWDRYDETQVNVFIKNTRNVNLPDEVFEPGEEKPKSSKKRSYNDAGNNNETMKRQKISTA

SEQ ID NO: 93 - protein, 608 aa, Exophiala spinifera
MAEKQWGMTPPMSTALPEPIDTEKTADLIEELKKENNYEPLEATQKRMATLGLLNRVTQEFVREVSRRRRMPPSQIEQFGGKIFPYGSYRLGVFGPGSDIDTLAVAPRHVTREDFFEYFPTVLKRMTAEGDISSLTPVPDSYVPIIKLVLNDIEIDLIFASIASLQTIPKNLTLNDNNLLTGLDQATIRAVTGPRVTDEILSLVPEQKTFRTALRAIKLWAQRRAVYANIVGYPGGAAWAMLVARVCQLYPHAVGATLVDKFFFVMKNWDWPTPVMLKDIEQPKPSQANEFKVWNPALYKGDKKMLMPIITPAFPSTSATYNISKSGKTVILRELERASQITNNIFAGKARWSDLFKKHSFFTADHKYYLGVTASTLNADSAKQWAGLVESKVRIFVMLLEGIPDITLARPFTKGFKRVHKCADETQIREVQKGSMKYKFEETKTVETTDPELVTSNGDGAAVPAANGAKPETDQGAHTVYTYTFYIGIDTTAKGSLNIAPSFQNFKDICEGWASFNRDSHFLTLASVKCWDLPDDVFDAKAGEVKPSRPVKKVAKQVKAEAKSGVRRSINEVEATETNGDAAKRQKLMTPTPTPTPTPAPKPTAAPA

SEQ ID NO: 94 - protein, 625 aa, Scedosporium apiospermum
MADRQLGVTPPISMALPTPAEIEANNAMVEELRKQGIFESKEETDKRNLVLESLQKMCDEFVTRVAQEKDQGLARDARGKIFTYGGFRLGVFGPGSDIDTLVVAPKYVTRDDYFAIFPDLLLEMAPKGAITGLAVVTDAFVPIIKFEYWGISIDMIFSRIAMLKKLPTNTSQFNLTDTALLRGLDETEIRSLNGTRVAHEILDLVPEQSTFQMALRAIKLWAQRRAIYANVMGYPGGAAWAMLVARVCQLYPRATAATIVSKFFCIMRQWPWPQPVLLKHIERAPLGYRIWNPVVYPSDKHHLMPIITPAYPSTNAAYNINRSSMSIIQTELSRADGITDGIVVGQRPWSDLFEKHTFFTADYKYYLAIVSSGITKDAHKKWSGFVESKVRMLVLALDRHDTIALAQAFVKGYDRVHKCNADSEIQKVQQGDLAYMIKEEDAPKEEQTSPVKSETKPDPGANEKNSAGGSNSNGDVAGSADAVKNEEGANGVKAGDENVYIYTTTHYIGLALRPGQKSINLANEVGEFKKLCKGWEKFDHQLNAITVQHLRNSDLPDDVFAEGEVKPRKRVVRKANGTGPSPPVDAKQATNGTTSQVDSKKRAAAETGAAAPAAKRRQPASVVAA

SEQ ID NO: 95 - protein, 612 aa, Trichoderma citrinoviride
MAEQNYGVTPPISVTLPTEAENRASDALLEELRRQKTFESPSDTEKRHEICNEFVRKVAREREPKNEILIKNARGKVFTYGSFRLGVFGPGSDIDTLIVAPKYVTREDYFNYFPDLLVSMAPPGAITDLTVVKDAFVPIIKFEYSGISIDLIFSRIIQKQIAPDFHSLKDSSLLRGLDEAELRSLNGTRVTDEILELVPEKSTFKLALRAIKLWAQRRAVYANIMGFPGGAAWAMLVARVCQLYPKATMSVIVNKFFLVIGQWRWPQPVLLKPIEGGPLPVRVWNPKVYKGDSFHLMPVITPAYPSTCATFNITRSSMTIIQRELRRGLEISEQIMVGKRPWSDLFVKHTFFTSGYRYYISVVSASKDKEAHKVWSGYVESKIRMLVQKLEQHPSIALAHAFNKGYDRRHLCRNEQEIGQVQEGSLEFMIKDWDDNKLNGAAIKPEKVEGDSKPNGADIIQPEKVEGESKLSDIPVKPEMDEAESKLSEVAIKTETKDGSTESVFPVEVYTTTHYIGLELEEGAKSLDLSYQVDEFKVLCTSWKKYQEDLEHLVSLGVQHVRNFNLPDDVFEPGEQKPQKKSSAKSLANKKRGAPEDNTPPAKRQQASVAAA

SEQ ID NO: 96 - protein, 598 aa, Aspergillus thermomutatus
MSAPPVRQWGVTPPISTALPTPDELAANDDLISELKAQNNFESPAETERRKQVLQLIQRVTHEFVKVVSRKKGLSPAAVEAAGGKIFTYGSYRLGVYGPGSDIDTLVVGPKHVLIDDFFSDFPPVLEKMAPPGAIEKMTPVPDAFVPIIKLELSGISIDLIFARLIVSSVPLNLDLKNNDYLRGLDEKEVRSLNGTRVTDEILELVPQQKTFRLALRAIKLWAQRRAIYSNIVGFPGGAAWAMLVARVCQLYPHATGSVIVGKFFRIMNKWAWPQPVLLKPIEDGPLQIKVWNPKIYHGDRFHLMPIITPAYPSTCATHNISMSTKAVILRELQRGGDIVDKIFLKQLTWNDLFARHSFFTHDYKYYLSITASSRTKEAESVWSGLVESKIRHLVGALDRKPTIAVAHPFPKGFERVHIISNEEEAEAVKNGSTKYQDKGTKTETTDETKDAAHQAAAQSGVENAEVEPVGENANGDSRIIYTTTYYIGLELKPLEPGASRSLDISTDAQIFKSTCTSWAGYQPGINDLSITHVRNFDLPEDVFQPGEARPTRPKKKVIKKPEAGAQKRGIDSLDDASLPAAKRQVTSNGISSTPTPA

SEQ ID NO: 97 - protein, 578 aa, Sodiomyces alkalinus
  source 1..578: note = F11
MQDRPLGVTPPISTSLPTDAEHAVNESLLEELKRQNTFESPAETAKREKVLEQLQLICNEFVRRVAREREAGSEALIKEARGRIFTYGSFRLGVYGPGSDIDALVVAPKYVTRDDYFKLFPGLLQEIPPTGAITDLAVVSDAFVPIIKFDFLGISIDLIFSRIASLKQLPKDKDWNLKDSNILRGLDEAELRSLNGTRVTDEIISLVPEPSTFRLALRAIKLWAQRRAVYANIMGFPGGAAWAMLVARVCQLYPKATSSVVVNKFFHIMRRWPWPQPVLLKAVESGPLQVRVWNPKLYKGDQFHLMPIITPAYPSTCATYNITKSAMTVIQRELQRGCEITDSVMMSKQPWSDLFVKHAFFTSDYKHYISVITTSTTKEAHKIWSGYVESKVRVLVQGLEQHPSIALAHAFNKGYDRRHKCSSEQEINQVQEGSLDYLLKAGDSSAGDPAEGNKSPRRPADGEHPEITPEATETIVFTTTHYIGLELVEGAKSLDLSYQVDSFKQLCGQWEKFNSNLNYLSVQHVRNVKLPDDVFEPCETRPQKKSATNGSLQKKKRGASEVDMHPPAKRQQSSVTAA

SEQ ID NO: 98 - protein, 606 aa, Neohortaea acidophila
MDPKLGVSGTMSMEPPSPKDIKLDDALLQELKSRNEFEAPEETQRRHSVLDRLESVLKRLVAMVGKQQGLPPGILNEAGGKIFTFGSFELGVYGPKSDMDTLMAAPKHVSREDFFQYMPDLLRKEFKPEEIAELTPVPGISVPIIKLELCGVSVDLIFCRLHLQSVPKSQELSNLDLLRGLDDTDLKCVNGTRVTRRILELVPQTKVFRMALRAVKLWAKQRALYGNIVGYPGGAAYAMMVARICQLYPRAAAPLVIWKFFYLMRKWNWPSPVLLQNHEEGSINLREWDPSIYPGDKRHLMPIITPAFPRANACHTIGPSTKKVLLQEMERAEGIVRSIYESGRPWRDLFQRHSFFTDAYRHYICVITAGRTKEAQQAWSGLVESKVKWLIVGIEHSDAKSVELVQPYNKGFNRVHECKGDADIDKTLDGNLDCQVKEIKTVTTEQAGDVQFQAAAQTDTDGQEVPAPNGEAEVPPQTDGPQTIWTTTFYLGIGLTKGANSLDISSPIRDFTQQCKEWQNYDENLHSIRVKHMRNYDLPADVFAEGETRPTRTKKKSAPKTTDPTAMDAANKKRSFNNSGLDTLDDPAKRRASANGTATPNGVPPR

SEQ ID NO: 99 - protein, 631 aa, Chaetomium sp.
  source 1..631: note = MPI-CAGE-AT-0009
MAEIIYGVTPPISTALPTEGEKRLNNALHQELRAQGTFESPQETEKRREVLRQLEKITNAFVQRAAAEKEPKNTFMIRDAIGRVFTYGSYRLGVYGPGSDMDTLVVAPKYVTVEQYFKIFPEVLVEMAPPGAITDLTPVPEAFVPIIKFEFSGISIDLIFCSIQTLKQLPDEKNWSLADNNLLRGLSENEVRSVNGTRVTDDILHLVPEPATFKLALRAVKLWAQRKAIYANIMGYPGGVAWAMLVARVCQLYPKATSAVVVNKFFHIMFKWPWPLPVLLKDIEYGCPVTRVAVWNPKIYASDRNHRMPIITPSYPSMCATHNIGRSSMAVIKQELEKGIQVTEDIMLGKRQWKDLFTKHTFFTSGFRYYLTVIASSRTKKAQNVWSGFVESRVRLLVNKLEMHPSIALARPFNKGYDREHRCKDNAQLEEVVSLGSLAYVYKPPADDEDKAKAEPKIEPKREVKQAIKTEDGRPAAEEEKPEARGENGMRIKQEHSDATQFPPPLDVRPEPSDETDGPVKLADIPEKKKDEPEDMEIYTTNHYIGLQMVEGSKSLDLSREVNDWKAMCMSNELYDEGLMFLSIQHLKNTGLPDDVFEPGEIKPRPVKKSLKRNAPEEPGNKQAQPPAKRH

SEQ ID NO: 100 - protein, 618 aa, Staphylotrichum longicolle
MGERFYGVTPPISTALPSEAEKRLNQALHQELRAQGTFESPAETEKRAEVLRKLEKITNVFVQRAAQEKEPKNTILIRDAIGRVFTYGSYRLGVYGPGSDMDTLVVAPKYVTVEQYFKIFPEVLVEMAPPGAITDLTPVPEAFVPIIKFEFSGISIDLIFCSIQTLKQLPDDKDWSLADNNLLRGLSENEVRSLNGTRVTDEILHLVPEPATFKLALRAIKLWAQRKAIYANIMGYPGGVAWAMLVARVCQLYPKAASAVIVNKFFNIMLKWPWPLPVLLKDIEYGSPVTRVAVWNPKLYASDRNHKMPIITPSYPSMCATHNVGKSSLVVIQQELERGAQVTDEIMIGKRPWKDLFTKHTFFTSGFRYYLTVIASSRTKKAQNAWSGFVESRVRVLVNKLELHPSIALARPFNKGYDREHRCKNNAQVEEVISAGSLAYVYKPPADGEEQAKTEPLQEVKPDKAEAATNVKSEPTEEHGDAKVKLEDIPEKKTEPEDMEIYHTNHYIGLQLVEGAKSLDLSSQVNDWKAMCTSNELYEEGLMFLSIQHIKNTSLPDDVFEPGEVKPRPVKKSLKRVASEEPSGKQMQPPAKRQVQGKAPVPAQRQQQQPTSTATAAA

SEQ ID NO: 101 - protein, 610 aa, Madurella mycetomatis
MAERIYGVTPPISTALPTEAEKRMHDALHKELRAQGTFESPAETEERKKVIKELEKITNEFVKRAAREKEPKNTILIRDAIGRVFTYGSYRLGVYGPGSDMDTLVVAPKYVTVEQYFRIFPAVLVEMAPPGAITDLTPVPEAFVPIIKFEFSGISIDLIFCSIQTLKQLPAEKGWSLADNNLLRGLSENEVRSLNGTRVTDEILNLVPEPATFKLALRAIKLWAQRKAIYANIMGYPGGVAWAMLVARVCQLYPKATSAVIVNKFFNIMFKWPWPLPVLLKDIEYGCPVTRVAVWNPKLYASDRNHKMPIITPAYPSMCATHNVGRSSMVVIQQELEKGVQITDEIMVGRRPWKDLFTKHTFFTSGFRYYLTVISSSRTKKAQNAWSGFIESRVRLLVNRLEMHPSIALARPFNKGYDRMHRCKDDTQVEEVVSAGSLAYLYKPPTEEDEKLKTEVKAEDGAPVKSEDGVPIKQEKAEDTSIQTVQGETSGDVKIKPEDTPEQKETEIEIYTTNHYIGLQLAEKAKSLDLSREVNDWKAMCMSNELYDADLMFLSIQNIKNTALPDDVFEPGEQKPQPPLKKGVKRMASEDPSAKQHQPPAKRQVQGKQP

SEQ ID NO: 102 - protein, 629 aa, Chaetomium globosum
MAEIIYGVTPPISTALPTEPEKRLNHALHQELRAQGTFESPEETEKRKEVLRQLEKITNVFVQRAAAQKEPKNTFMIRDAIGRVFTYGSYRLGVYGPGSDMDTLVVAPKYVTVEQYFEIFPEVLVEMAPPGAITDLTPVPEAFVPIIKFEFSGISIDLIFCSIQTLKQLPDEKNWSLADNNLLRGLSENEVRSVNGTRVTDDILHLVPEPATFKLALRAIKLWAQRKAIYANIMGYPGGVAWAMLVARVCQLYPKATSAVVVNKFFHIMFKWPWPLPVLLKDIEYGSPVTRVAVWNPKIYTSDRNHKMPIITPSYPSMCATHNIGTSSMIVIKQELENGIQVTEDIMLGKRQWKDLFTKHTFFTSGFRYYLTVIAASRTKKAQNVWSGFVESRVRLLVNRLEGHHSIALARPFNKGYDREHRCKNNTQLEEVVSVGSLAYVYKPPADSEEKAKAEPKVELKQEIKAEEKPETKGQDGLRIKQEYSESTQPPPPSNVKSEPNQDTNSEVKLEDIPEKKKDEPDEMEIYTTNHYIGLQLVEGAKSLDLSREVNDWKAMCTSNELFDEGLMFLSIQHLKNTNLPDDVFEPGETKPRPVKKSLKRAASEGPGNMHPQPPAKRHAQGKPSSHSA

SEQ ID NO: 103 - protein, 649 aa, Chaetomium sp.
  source 1..649: note = MPI-SDFR-AT-0129
MGERIYGVTPPISTALPTEQEKRLNNALHQELRAQGTFESPAETEKRREVLRHLEKITNVFVQKAAEKKEPKNTILIRDAIGRVFTYGSYRLGVYGPGSDMDTLVVAPKYVTVKEYFEIFPQVLVEMAPPGAITDLTPVPEAFVPIIKFEFSGISIDLIFCSIQSLKQLPDDKNWSLADNNLLRGLSENEVRSLNGTRVTDDILNLVPEPATFKLALRAIKLWAQRKAIYANIMGYPGGVAWAMLVARVCQLYPKATSAVVVNKFFHIMCKWPWPLPVLLQEIEYGCPVTRVAVWNPKIYPSDRHHKMPIITPSYPSMCATHNIGKSSMAVIKEELGAGVQVTEDIMLGKRQWKDLFTKHTFFTSGFRYYLTVIASSRTKEAQNVWSGFVESRVRLLVNKLEMHPSIALARPFNKGYEREHRCKNNAQVEDVVFTGSLAHVYKPPADAENPVKAEPKQESKTAANGMPAKEEQNPQVKTENGVPMKQDPSETAPPPATTPESKPNIKDEPADLNGSSHDDKDDIKLHDIPEMKKPEPEEMEMYTTNHYIGLQLVEGARSLDLSREVNDWKAMCTSNELYKEGKMFLSIQHIKNTALPDDVFEPGETKPKPVKKSLKRLASEEPGNRQSLPPAKRPTPAAGNKPPLPRPT

SEQ ID NO: 104 - protein, 632 aa, Neurospora tetrasperma
  source 1..632: note = FGSC 2508
MANPEHTLGVTPPISTALPTDAEKRQTEALLKELRAQGTFESQAETDKRWAVLADLQRITDEFVKRAAQEKEPHNAILIRDARGRIFTYGSFRLGVYGPGSDIDTLVVVPKYVTVKQYFDIFPNLLVEMAPPGAITDLTPVPEAFVPIIKFEYSGISIDLIFCSIQSLRQLPDDKDWNLNSNNLLRGLSENEVRSLNGTRVTDEILALVPEEKTFKLALRAIKLWAQRRAIYANIMGYPGGVAWAMLVARVCQLYPKATSAVIVRKFFTIMLNWPWPLPVLLKNIEYNQSITRVAVWNPKIYPSDRNHKMPIITPSYPSMCATHNVGRSSMAVIQQELERGVQISEEIMLGKRPWKDLFEKHTFFTNGFKFYLTVISSGRTKEAQNTWSGFVESRVRVLVQKLELHPSIALARPFNKGYDRVHRCKTDAQVEEIQTGNLTYMVKPTDTAANGNAKNDSKVEIKAEIKTEDGVAPEPAVPVVKEEPNDVVHLGTVPLKPEDNSVKPEANDYDEDKVKLEDIPEKEPELEIYTTNHYIGLQLVEGAKSLDLSREVDDFKAMCTSNDIFKAELMGLTIQHVRNFDLPDDVFEPGETKPVRPVKKKKRMAEGEHANGPMAKRQQMATAPPTIKSEH

SEQ ID NO: 105 - protein, 632 aa, Neurospora crassa
  source 1..632: note = OR74A
MGNPEQTLGVTPPISTTLPTDAEKRQTEALLKELRAQGTFESQAETDKRWAVLADLQRITDEFVKRAAQEKEPHNAILIRDARGRIFTYGSFRLGVYGPGSDIDTLVVVPKYVTVKQYFDIFPNLLVEMAPPGAITDLTPVPEAFVPIIKFEYSGISIDLIFCSIQSLRQLPDDKDWNLNSNNLLRGLSENEVRSLNGTRVTDEILALVPEEKTFKLALRAIKLWAQRRAIYANIMGYPGGVAWAMLVARVCQLYPKATSAVIVRKFFTIMLNWPWPLPVLLKNIEYNQSITRVAVWNPKIYPSDRNHKMPIITPSYPSMCATHNVGRSSMAVIQQELERGVQISEEIMLGKRPWKDLFEKHTFFTNGFKFYLTVISSGRTKEAQNTWSGFVESRVRVLVQKLELHPSIALARPFNKGYDRVHRCKTDAQVEEIQTGNLTYMVKPTDIAANGSAKNDSKVEVKAEIKTEDGVAREPTVPAVKEEPNDVVHLGTVPLKPEDNSVKPEANDYDEDKVKLEDIPEKEPELEIYTTNHYIGLQLVEGAKSLDLSREVDDFKAMCTSNDIFKAELMGLTIQHVRNFDLPDDVFEPGETKPVRPVKKKKRMAEGEHANGPMAKRQQMATAPPTIKSEH

SEQ ID NO: 106 - protein, 632 aa, Sordaria macrospora
  source 1..632: note = k-hell
MANPEHSLGVTPPISTALPTDAEKRQTDALFKELRAQGTFESQAETEKRWAVLADLQRITDEFVKRAAQESEPHNSILIRDARGRIFTYGSFRLGVYGPGSDIDTLVVVPKYVTVKQYFDIFPKLLEEMAPPGAITDLTPVPEAFVPIIKFEYSGISIDLIFCSIQSLRQLPDDKEWNLNNNNLLRGLSENEVRSLNGTRVTDEILALVPEEKTFKLALRAIKLWAQRRAIYANIMGYPGGVAWAMLVARVCQLYPKATSAVVVRKFFTIMLNWPWPLPVLLKNIEYNQSITRVAVWNPKIYPSDRNHKMPIITPSYPSMCATHNVGRSSMAVIQQELERGVQIAEEIMLGKRPWKDLFEKHTFFTNGFKFYLTVISSGRTKEAQNTWSGFVESRVRVLVQKLELHPSIALARPFNKGYDRVHRCKTDTQVEEIQTGNLSYMVKPTDTATNGNAKDESKVEVKAEIKKADGVAPDSAVPTVKEEPNDVVHLGTVPLKPEETPVKPEAKDEDDGKVKLADIPEKELEIEIYTTNHYIGLQLVEKAKSLDLSREVDDFKAMCTSNDIFKAELMGLTIQHVRNFDLPDDVFEPGETKPTKPVKKKKRMAEGDPANGPMAKRQQMATAPPTIKSEQ

SEQ ID NO: 107 - protein, 650 aa, Podospora anserina
  source 1..650: note = S mat+
MAERVYGVTPPISTALPTEAEKRMHQALHEELRAQGTFESRAETEARKKVIAQLEKITTAFVKKATAETQNSFMVREAIGRVFTYGSYRLGVYGPGSDIDTLVVAPKHVTTEQYFRLFPPLLVEMAPPGAITDLTPVPDAFVPIIKFEFSGISIDLIFCSIKGLTQIPEDKSWNLTDNTLLRGLSENEVRSLNGTRVTDEILNLVPEPATFKLALRAIKLWAQRKAIYANIMGFPGGVAWAMLVARVCQLYPKATSAVVVNKFFHIMLKWPWPLPVLLKDIEYNISVTRAPVWNPKLYSSDRNHRMPIITPAYPAMCATHNINRSSKVVIMQELEKGVQVTEEIMTGRAPWKALFTKHTFFTSDFRYYLTVISSSRTKDAQNVWSGFIESRVRVLVNKIENHAAIVLARPFNKGYDRQHRCQNYEQLGDVVNSGSLAYLYKPTPEEEEKAKGEAKTEVKAELINANLKPESVLQDGPVVKPEPGTETAAPPPVTAADGAAIKPEPGAESAMPPPAPPPVADGVKQEEVTKNGDGVEQKDGNDGEVKLADIPEKKQELEVFTTNYYIGLQLGKTPASLDLSREVQEWMSMCRSSDLYKEGVNFLAVTHIKNTMLPDDVFEPGEVRPRPPKKKLKRQAPDDSSAQQPTKKSK

SEQ ID NO: 108 - protein, 650 aa, Podospora comata
MAERVYGVTPPISTALPTEAEKRMHQALHEELRAQGTFESRAETEARKKVIAQLEKITTAFVKKATAETQNSFMVREAIGRVFTYGSYRLGVYGPGSDIDTLVVAPKHVTTEQYFRLFPPLLVEMAPPGAITDLTPVPDAFVPIIKFEFSGISIDLIFCSIKGLTQIPEDKSWNLTDNTLLRGLSENEVRSLNGTRVTDEILNLVPEPATFKLALRAIKLWAQRKAIYANIMGFPGGVAWAMLVARVCQLYPKATSAVVVNKFFHIMLKWPWPLPVLLKDIEYNISVTRAPVWNPKLYSSDRNHRMPIITPAYPAMCATHNINRSSKVVIMQELEKGVQVTEEIMTGRAPWKALFTKHTFFTSDFRYYLTVISSSRTKDAQNVWSGFIESRVRVLVNKIENHAAIVLARPFNKGYDRQHRCQNYEQLGDVVNSGSLAYLYKPTPEEEEKAKGEAKTEVKAELINANLKPESVLQDGPVVKPEPGTETAAPPPVTTADGAAIKPEPGAESAMPPPAPPPVADVVKKEEVTKNGDGVEQKDGNDGEVKLADIPEKKQELEVFTTNYYIGLQLGKTPASLDLSREVQEWMSMCRSSDLYKEGVNFLAVTHIKNTMLPDDVFEPGEVRPRPPKKKLKRQAPDDSSAQQPTKKSK

SEQ ID NO: 109 - protein, 21 aa, synthetic construct
  REGION 1..21: note = Exemplary Tag
GSSGENLYFQGSSGSHHHHHH

SEQ ID NO: 110 - protein, 524 aa, synthetic construct
MAERTYGVTPPISTALPTEQEKALNKALHDELRAQGTFESRAETEKRKEVLAQLEKITNAFVQRAAREKHPKNAILIRDAIGRVFTHGSYRLGVYGPGSDMDTLVVAPKYVTEEQYFRIFPEILVEMAPPGAITDLTPVPEAFVPIIKFEFSGISIDLIFCSIQTLEQLPADKSWSLADNNLLRGLSETAVRSLNGTRNSDEILRLVPEPATFKLALRAIKLWAQRKAIYANIMGYPGGAAWAVLVARVCQLYPKATSAVIVNKFFNIMLKWPWPLPVLLKDIEYNGPVTRVRVWNPKLYASDRNHKMPIITPAYPSTCCTHNVGRSSMVVIQQELKKGAEVTEEIMLGRRPWKDLFTKHTFFTSGFKYYLTVISSSRTKKAQNVWSGFIESRVRLLVNKIEMHPSIALVRPFNKGYDRMHRCKNDAQVEEVVSAGSLAYVYTPPAGGGSGGGSMEIYTTNHYIGLQLVEGAKSLDLSREVNDWKAMCMSNELYEEGLMFLSIQHLKNTALPDDVFEPGEVKPR

SEQ ID NO: 111 - protein, 524 aa, synthetic construct
MAERTYGVTPPISTALPTEQEKALNKALHDELRAQGTFESRAETEKRKEVLAQLEKITNAFVQRAAREKHPKNAILIRDAIGRVFTHGSYRLGVYGPGSDIDTLVVAPKYVTEEQYFRIFPEILVEMAPPGAITDLTPVPEAFVPIIKFEFSGISIDLIFCSIQTLEQLPADKSWSLADNNLLRGLSETAVRSLNGTRNSDEILRLVPEPATFKLALRAIKLWAQRKAIYANIMGYPGGAAWAVLVARVCQLYPKATSAVIVNKFFNIMLKWPWPLPVLLKDIEYNGPVTRVRVWNPKLYASDRNHKMPIITPAYPSTCCTHNVGRSSMVVIQQELKKGAEVTEEIMLGRRPWKDLFTKHTFFTSGFKYYLTVISSSRTKKAQNVWSGFIESRVRLLVNKIEMHPSIALVRPFNKGYDRMHRCKNDAQVEEVVSAGSLAYVYTPPAGGGSGGGSMEIYTTNHYIGLQLVEGAKSLDLSREVNDWKAMCMSNELYEEGLMFLSIQHLKNTALPDDVFEPGEVKPR

SEQ ID NO: 112 - protein, 524 aa, synthetic construct
MAERTYGVTPPISTALPTEQEKALNKALHDELRAQGTFESRAETEKRKEVLAQLEKITNAFVQRAAREKHPKNAILIRDAIGRVFTHGSYRLGVYGPGSDMDTLVVAPKYVTEEQYFRIFPEVLVEMAPPAAITDLTPVPEAYVPIIKFEFSGISIDLGFCSIQTLEQLPADKSWSLADNNLLRGLSENAVRSLNGTRNSDEILRLVPEPATFKLALRAIKLWAQRKAIYANIMGYPGGAAWAVLVARVCQLYPKATSAVIVNKFFNIMLKWPWPLPVLLKDIEYNGPVTRVRVWNPKLYASDRNHKMPIITPAYPSTCCTHNVGRSSMVVIQQELKKGAEVTEEIMLGRRPWKDLFTKHTFFTSGFKYYLTVISSSRTKKAQNVWSGFIESRVRLLVNKIEMHPSIALVRPFNKGYDRMHRCKNDAQVEEVVSAGSLAYVYTPPAGGGSGGGSMEIYTTNHYIGLQLVEGAKSLDLSREVNDWKAMCMSNELYEEGLMFLSIQHLKNTALPDDVFEPGEVKPR

SEQ ID NO: 113 - protein, 524 aa, synthetic construct
MAERTYGVTPPISTALPTEQEKALNKALHDELRAQGTFESRAETEKRKEVLAQLEKITNAFVQRAAREKHPKNAILIRDAIGRVFTHGSYRLGVYGPGSDMDTLVVAPKYVTEEQYFRIFPEILVEMAPPGAITDLTPVPEAFVPIIKFEFSGISIDLIFCSIQTLKQLPADKSWSLADNNLLRGLSETAVRSLNGTRNSDEILRLVPEPATFKLALRAIKLWAQRKAIYANIMGYPGGAAWAVLVARVCQLYPKATSAVIVNKFFNIMLKWPWPLPVLLKDIEYNGPVTRVRVWNPKLYASDRNHKMPIITPAYPSTCCTHNVGRSSMVVIQQELKKGAEVTEEIMLGRRPWKDLFTKHTFFTSGFKYYLTVISSSRTKKAQNVWSGFIESRVRLLVNKIEMHPSIALVRPFNKGYDRMHRCKNDAQVEEVVSAGSLAYVYTPPAGGGSGGGSMEIYTTNHYIGLQLVEGAKSLDLSREVNDWKAMCMSNELYEEGLMFLSIQHLKNTALPDDVFEPGEVKPR

SEQ ID NO: 114 - protein, 524 aa, synthetic construct
MAERTYGVTPPISTALPTEQEKALNKALHDELRAQGTFESRAETEKRKEVLAQLEKITNAFVQRAAREKHPKNAILIRDAIGRVFTHGSYRLGVYGPGSDMDTLVVAPKYVTEEQYFRIFPEILVEMAPPGAITDLTPVPEAYVPIINFEFSGISIDLIFCSIQTLEQLPADKSWSLADNNLLRGLSETAVRSLNGTRNSDEILRLVPEPATFKLALRAIKLWAQRKAIYANIMGYPGGAAWAVLVARVCQLYPKATSAVIVNKFFNIMLKWPWPLPVLLKDIEYNGPVTRVRVWNPKLYASDRNHKMPIITPAYPSTCCTHNVGRSSMVVIQQELKKGAEVTEEIMLGRRPWKDLFTKHTFFTSGFKYYLTVISSSRTKKAQNVWSGFIESRVRLLVNKIEMHPSIALVRPFNKGYDRMHRCKNDAQVEEVVSAGSLAYVYTPPAGGGSGGGSMEIYTTNHYIGLQLVEGAKSLDLSREVNDWKAMCMSNELYEEGLMFLSIQHLKNTALPDDVFEPGEVKPR

SEQ ID NO: 115 - protein, 524 aa, synthetic construct
MAERTYGVTPPISTALPTEQEKALNKALHDELRAQGTFESRAETEKRKEVLAQLEKITNAFVQRAAREKHPKNAILIRDAIGRVFTHGSYRLGVYGPGSDMDTLVVAPKYVTEEQYFRIFPEILVEMAPPAAITDLTPVPEAYVPIIKFEFSGISIDLIFCSIQTLIQLPADKSWSLADNNLLRGLSENAVRSLNGTRNTDEILRLVPEPATFKLALRAIKLWAQRKAIYANIMGYPGGAAWAVLVARVCQLYPKATSAVIVNKFFNIMLKWPWPLPVLLKDIEYNGPVTRVRVWNPKLYASDRNHKMPIITPAYPSTCCTHNVGRSSMVVIQQELKKGAEVTEEIMLGRRPWKDLFTKHTFFTSGFKYYLTVISSSRTKKAQNVWSGFIESRVRLLVNKIEMHPSIALVRPFNKGYDRMHRCKNDAQVEEVVSAGSLAYVYTPPAGGGSGGGSMEIYTTNHYIGLQLVEGAKSLDLSREVNDWKAMCMSNELYEEGLMFLSIQHLKNTALPDDVFEPGEVKPR

SEQ ID NO: 116 - protein, 524 aa, synthetic construct
MAERTYGVTPPISTALPTEQEKALNKALHDELRAQGTFESRAETEKRKEVLAQLEKITNAFVQRAAREKHPKNAILIRDAIGRVFTHGSYRLGVYGPGSDMDTLVVAPKYVTEEQYFRIFPEVLVEMAPPAAITDLTPVPEAYVPIIKFEFSGISIDLIFCSIQTLKQLPADKSWSLADNNLLRGLSENAVRSLNGTRNTDEILRLVPEPATFKLALRAIKLWAQRKAIYANIMGYPGGAAWAVLVARVCQLYPKATSAVIVNKFFNIMLKWPWPLPVLLKDIEYNGPVTRVRVWNPKLYASDRNHKMPIITPAYPSTCCTHNVGRSSMVVIQQELKKGAEVTEEIMLGRRPWKDLFTKHTFFTSGFKYYLTVISSSRTKKAQNVWSGFIESRVRLLVNKIEMHPSIALVRPFNKGYDRMHRCKNDAQVEEVVSAGSLAYVYTPPAGGGSGGGSMEIYTTNHYIGLQLVEGAKSLDLSREVNDWKAMCMSNELYEEGLMFLSIQHLKNTALPDDVFEPGEVKPR

SEQ ID NO: 117 - protein, 524 aa, synthetic construct
MAERTYGVTPPISTALPTEQEKALNKALHDELRAQGTFESRAETEKRKEVLAQLEKITNAFVQRAAREKHPKNAILIRDAIGRVFTHGSYRLGVYGPGSDMDTLVVAPKYVTVEQYFRIFPEVLVEMAPPAAITDLTPVPEAYVPIIKFEFSGISIDLIFCSIQTLIQLPADKSWSLADNNLLRGLSENAVRSLNGTRNTDEILRLVPEPATFKLALRAIKLWAQRKAIYANIMGYPGGAAWAVLVARVCQLYPKATSAVIVNKFFNIMLKWPWPLPVLLKDIEYNGPVTRVRVWNPKLYASDRNHKMPIITPAYPSTCCTHNVGRSSMVVIQQELKKGAEVTEEIMLGRRPWKDLFTKHTFFTSGFKYYLTVISSSRTKKAQNVWSGFIESRVRLLVNKIEMHPSIALARPFNKGYDRMHRCKNDAQVEEVVSAGSLAYVYTPPAGGGSGGGSMEIYTTNHYIGLQLVEGAKSLDLSREVNDWKAMCMSNELYEEGLMFLSIQHLKNTALPDDVFEPGEVKPR

SEQ ID NO: 118 - protein, 524 aa, synthetic construct
MAERTYGVTPPISTALPTEQEKALNKALHDELRAQGTFESPAETEKRKEVLAQLEKITNAFVQRAAREKHPKNAILIRDAIGRVFTHGSYRLGVYGPGSDMDTLVVAPKYVTVEQYFRIFPEVLVEMAPPGAITDLTPVPEAYVPIIKFEFSGISIDLIFCSIQTLIQLPADKSWSLADNNLLRGLSENAVRSLNGTRNTDEILRLVPEPATFKLALRAIKLWAQRKAIYANIMGYPGGAAWAVLVARVCQLYPKATSAVIVNKFFNIMLKWPWPLPVLLKDIEYNGPVTRVRVWNPKLYASDRNHKMPIITPAYPSTCCTHNVGRSSMVVIQQELKKGAEVTEEIMLGRRPWKDLFTKHTFFTSGFKYYLTVISSSRTKKAQNVWSGFIESRVRLLVNKIEMHPSIALARPFNKGYDRMHRCKNDAQVEEVVSAGSLAYVYTPPAGGGSGGGSMEIYTTNHYIGLQLVEGAKSLDLSREVNDWKAMCMSNELYEEGLMFLSIQHLKNTALPDDVFEPGEVKPR

SEQ ID NO: 119 - protein, 524 aa, synthetic construct
MAERTYGVTPPISTALPTEQEKALNKALHDELRAQGTFESPAETEKRKEVLAQLEKITNAFVQRAAREKHPKNAILIRDAIGRVFTHGSYRLGVYGPGSDMDTLVVAPKYVTVEQYFRIFPEVLVEMAPPGAITDLTPVPEAYVPIIKFEFSGISIDLIFCSIQTLIQLPADKSWSLADNNLLRGLSENAVRSLNGTRNTDEILRLVPEPATFKLALRAIKLWAQRKAIYANIMGYPGGAAWAVLVARVCQLYPKATSAVIVNKFFNIMLKWPWPLPVLLKDIEYNGPVTRVRVWNPKLYASDRNHKMPIITPAYPSTCCTHNVGRSSMVVIQQELKKGASVTEEIMLGRRPWKDLFTKHTFFTSGFKYYLTVISSSRTKKAQNVWSGFIESRVRLLVNKIEMHPSIALARPFNKGYDRMHRCKNDAQVEEVVSAGSLAYVYTPPAGGGSGGGSMEIYTTNHYIGLQLVEGAKSLDLSREVNDWKAMCMSNELYEEGLMFLSIQHLKNTALPDDVFEPGEVKPR

SEQ ID NO: 120 - protein, 524 aa, synthetic construct
MAERTYGVTPPISTALPTEQEKALNKALHDELRAQGTFESPAETEKRKEVLAQLEKITNAFVQRAAREKHPKNAILIRDAIGRVFTHGSYRLGVYGPGSDMDTLVVAPKYVTVEQYFRIFPEVLVEMAPPGAITDLTPVPEAYVPIIKFEFSGISIDLIFCSIQTLIQLPADKSWSLADNNLLRGLSENAVRSLNGTRNTDEILRLVPEPATFKLALRAIKLWAQRKAIYANIMGYPGGAAWAVLVARVCQLYPKATSAVIVNKFFNIMLKWPWPLPVLLKDIEYNGPATRVRVWNPKLYASDRNHKMPIITPAYPSTCCTHNVGRSSMVVIQQELKKGAEVTEEIMLGRRPWKDLFTKHTFFTSGFKYYLTVISSSRTKKAQNVWSGFIESRVRLLVNKIEMHPSIALARPFNKGYDRMHRCKNDAQVEEVVSAGSLAYVYTPPAGGGSGGGSMEIYTTNHYIGLQLVEGAKSLDLSREVNDWKAMCMSNELYEEGLMFLSIQHLKNTALPDDVFEPGEVKPR

SEQ ID NO: 121 - protein, 488 aa, synthetic construct
AERTYGVTPPISTALPTEQEKALNKALHDELRAQGTFESRAETEKRKEVLAQLEKITNAFVQRAAREKHPKNAILIRDAIGRVFTHGSYRLGVYGPGSDMDTLVVAPKYVTVEQYFRIFPEVLVEMAPPAAITDLTPVPEAYVPIIKFEFSGISIDLIFCSIQTLIQLPADKSWSLADNNLLRGLSENAVRSLNGTRNTDEILRLVPEPATFKLALRAIKLWAQRKAIYANIMGYPGGAAWAVLVARVCQLYPKATSAVIVNKFFNIMLKWPWPLPVLLKDIEYNGPVTRVRVWNPKLYASDRNHKMPIITPAYPSTCCTHNVGRSSMVVIQQELKKGAEVTEEIMLGRRPWKDLFTKHTFFTSGFKYYLTVISSSRTKKAQNVWSGFIESRVRLLVNKIEMHPSIALARPFNKGYDRGPGYTTNHYIGLQLVEGAKSLDLSREVNDWKAMCMSNELYEEGLMFLSIQHLKNTALPDDVFEPGEVKPR

SEQ ID NO: 122 - protein, 489 aa, synthetic construct
MAERTYGVTPPISTALPTEQEKALNKALHDELRAQGTFESRAETEKRKEVLAQLEKITNAFVQRAAREKHPKNAILIRDAIGRVFTHGSYRLGVYGPGSDMDTLVVAPKYVTVEQYFRIFPEVLVEMAPPAAITDLTPVPEAYVPIIKFEFSGISIDLIFCSIQTLIQLPADKSWSLADNNLLRGLSENAVRSLNGTRNTDEILRLVPEPATFKLALRAIKLWAQRKAIYANIMGYPGGAAWAVLVARVCQLYPKATSAVIVNKFFNIMLKWPWPLPVLLKDIEYNGPVTRVRVWNPKLYASDRNHKMPIITPAYPSTCCTHNVGRSSMVVIQQELKKGAEVTEEIMLGRRPWKDLFTKHTFFTSGFKYYLTVISSSRTKKAQNVWSGFIESRVRLLVNKIEMHPSIALARPFNKGYDRPNGYTTNHYIGLQLVEGAKSLDLSREVNDWKAMCMSNELYEEGLMFLSIQHLKNTALPDDVFEPGEVKPR

SEQ ID NO: 123 - protein, 491 aa, synthetic construct
MAERTYGVTPPISTALPTEQEKALNKALHDELRAQGTFESRAETEKRKEVLAQLEKITNAFVQRAAREKHPKNAILIRDAIGRVFTHGSYRLGVYGPGSDMDTLVVAPKYVTVEQYFRIFPEVLVEMAPPAAITDLTPVPEAYVPIIKFEFSGISIDLIFCSIQTLIQLPADKSWSLADNNLLRGLSENAVRSLNGTRNTDEILRLVPEPATFKLALRAIKLWAQRKAIYANIMGYPGGAAWAVLVARVCQLYPKATSAVIVNKFFNIMLKWPWPLPVLLKDIEYNGPVTRVRVWNPKLYASDRNHKMPIITPAYPSTCCTHNVGRSSMVVIQQELKKGAEVTEEIMLGRRPWKDLFTKHTFFTSGFKYYLTVISSSRTKKAQNVWSGFIESRVRLLVNKIEMHPSIALARPFNKGYDRHDGARYTTNHYIGLQLVEGAKSLDLSREVNDWKAMCMSNELYEEGLMFLSIQHLKNTALPDDVFEPGEVKPR

SEQ ID NO: 124 - protein, 524 aa, synthetic construct
MAERTYGVTPPISTALPTEQEKALNKALHDELRAQGTFESRAETEKRKEVLAQLEKITNAFVQRAAREKHPKNAILIRDAIGRVFTHGSYRLGVYGPGSDMDTLVVAPKYVTVEQYFRIFPEVLVEMAPPAAITDLTPVPEAYVPIIKFEFSGISIDLIFCSIQTLIQLPADKSWSLADNNLLRGLSENAVRSLNGTRNTDEILRLVPEPATFKLALRAIKLWAQRKAIYANIMGYPGGAAWAVLVARVCQLYPKATSAVIVNKFFNIMLKWPWPLPVLLKDIEYNGPVTRVRVWNPKLYASDRNHKMPIITPAYPSTCCTHNVGRSSMVVIQQELKKGAEVTEEIMLGRRPWKDLFTKHTFFTSGFKYYLTVISSSRTKKAQNVWSGFIESRVRLLVNKLEMHPSIALARPFNKGYDRMHRCKNDAQVEEVVSAGSLAYVYTPPAGGGSGGGSMEIYTTNHYIGLQLVEGAKSLDLSREVNDWKAMCMSNELYEEGLMFLSIQHLKNTALPDDVFEPGEVKPR

SEQ ID NO: 125 - protein, 524 aa, synthetic construct
MAERTYGVTPPISTALPTEQEKALNKALHDELRAQGTFESRAETEKRKEVLAQLEKITNAFVQRAAREKHPKNAILIRDAIGRVFTHGSYRLGVYGPGSDMDTLVVAPKYVTVEQYFRIFPEVLVEMAPPAAITDLTPVPEAYVPIIKFEFSGISIDLIFCSIQTLIQLPADKSWSLADNNLLRGLSENAVRSLNGTRNTDEILRLVPEPATFKLALRAIKLWAQRKAIYANIMGYPGGAAWAVLVARVCQLYPKATSAVIVNKFFNIMLKWPWPLPVLLKDIEYNGPVTRVRVWNPKLYASDRNHKMPIITPAYPSTCCTHNVGRSSMVVIQQELKKGAEVTEEIMLGRRPWKDLFTKHTFFTSGFKYYLTVISSSRTKQAQNVWSGFIESRVRLLVNKIEMHPSIALARPFNKGYDRMHRCKNDAQVEEVVSAGSLAYVYTPPAGGGSGGGSMEIYTTNHYIGLQLVEGAKSLDLSREVNDWKAMCMSNELYEEGLMFLSIQHLKNTALPDDVFEPGEVKPR

SEQ ID NO: 126 - protein, 524 aa, synthetic construct
MAERTYGVTPPISTALPTEQEKALNKALHDELRAQGTFESRAETEKRKEVLAQLEKITNAFVQRAAREKHPKNAILIRDAIGRVFTHGSYRLGVYGPGSDMDTLVVAPKYVTVEQYFRIFPEVLVEMAPPAAITDLTPVPEAYVPIIKFEFSGISIDLIFCSIQTLIQLPADKSWSLADNNLLRGLSENAVRSLNGTRNTDEILRLVPEPATFKLALRAIKLWAQRKAIYANIMGYPGGAAWAVLVARVCQLYPKATSAVIVNKFFNIMLKWPWPLPVLLKDIEYNGPVTRVRVWNPKLYASDRNHKMPIITPAYPSTCCTHNVGRSSMVVIQQELKKGAEVTEEIMLGRRPWKDLFTKHTFFTSGFKYYLTVISSSRTKKAQNVWRGFIESRVRLLVNKIEMHPSIALARPFNKGYDRMHRCKNDAQVEEVVSAGSLAYVYTPPAGGGSGGGSMEIYTTNHYIGLQLVEGAKSLDLSREVNDWKAMCMSNELYEEGLMFLSIQHLKNTALPDDVFEPGEVKPR

SEQ ID NO: 127 - protein, 491 aa, synthetic construct
MAERTYGVTPPISTALPTEQEKALNKALHDELRAQGTFESPAETEKRKEVLAQLEKITNAFVQRAAREKHPKNAILIRDAIGRVFTHGSYRLGVYGPGSDMDTLVVAPKYVTVEQYFRIFPEVLVEMAPPGAITDLTPVPEAYVPIIKFEFSGISIDLIFCSIQTLIQLPADKSWSLADNNLLRGLSENAVRSLNGTRNTDEILRLVPEPATFKLALRAIKLWAQRKAIYANIMGYPGGAAWAVLVARVCQLYPKATSAVIVNKFFNIMLKWPWPLPVLLKDIEYNGPATRVRVWNPKLYASDRNHKMPIITPAYPSTCCTHNVGRSSMVVIQQELKKGASVTEEIMLGRRPWKDLFTKHTFFTSGFKYYLTVISSSRTKKAQNVWSGFIESRVRLLVNKIEMHPSIALARPFNKGYDRHDGARYTTNHYIGLQLVEGAKSLDLSREVNDWKAMCMSNELYEEGLMFLSIQHLKNTALPDDVFEPGEVKPR

SEQ ID NO: 128 - protein, 491 aa, synthetic construct
MAERTYGVTPPISTALPTEQEKALNKALHDELRAQGTFESPAETEKRKEVLAQLEKITNAFVQRAAREKHPKNAILIRDAIGRVFTHGSYRLGVYGPGSDMDTLVVAPKYVTVEQYFRIFPEVLVEMAPPGAITDLTPVPEAYVPIIKFEFSGISIDLIFCSIQTLIQLPADKSWSLADNNLLRGLSENAVRSLNGTRNTDEILRLVPEPATFKLALRAIKLWAQRKAIYANIMGYPGGAAWAVLVARVCQLYPKATSAVIVNKFFNIMLKWPWPLPVLLKDIEYNGPATRVRVWNPKLYASDRNHKMPIITPAYPSTCCTHNVGRSSMVVIQQELKKGASVTEEIMLGRRPWKDLFTKHTFFTSGFKYYLTVISSSRTKKAQNVWRGFIESRVRLLVNKLEMHPSIALARPFNKGYDRHDGARYTTNHYIGLQLVEGAKSLDLSREVNDWKAMCKSNELYEEGLMFLSIQHLKNTALPDDVFEPGEVKPR

SEQ ID NO: 129 - protein, 491 aa, synthetic construct
MAERTYGVTPPISTALPTEQEKALNKALHDELRAQGTFESPAETEKRKEVLAQLEKITNAFVQRAAREKHPKNAILIRDAIGRVFTHGSYRLGVYGPGSDMDTLVVAPKYVTVEQYFRIFPEVLVEMAPPGAITDLTPVPEAYVPIIKFEFSGISIDLIFCSIQTLIQLPADKSWSLADNNLLRGLSENAVRSLNGTRNTDEILRLVPEPATFKLALRAIKLWAQRKAIYANIMGYPGGAAWAVLVARVCQLYPKATSAVIVNKFFNIMLKWPWPLPVLLKDIEYNGPATRVRVWNPKLYASDRNHKMPIITPAYPSTCCTHNVGRSSMVVIQQELKKGASVTEEIMLGRRPWKDLFTKHTFFTSGFKYYLTVISSSRTKKAQNVWRGFIESRVRLLVNKLEMHPSIALARPFNQGYDRHDGARYTTNHYIGLQLVEGAKSLDLSREVNDWKAMCKSNELYEEGLMFLSIQHLKNTALPDDVFEPGEVKPR

SEQ ID NO: 130 - protein, 491 aa, synthetic construct
MAERTYGVTPPISTALPTEQEKALNKALHDELRAQGTFESPAETEKRKEVLAQLEKITNAFVQRAAREKHPKNAILIRDAIGRVFTHGSYRLGVYGPGSDMDTLVVAPKYVTVEQYFRIFPEVLVEMAPPGAITDLTPVPEAYVPIIKFEFSGISIDLIFCSIQTLIQLPADKSWSLADNNLLRGLSENAVRSLNGTRNTDEILRLVPEPATFKLALRAIKLWAQRKAIYANIMGYPGGAAWAVLVARVCQLYPKATSAVIVNKFFNIMLKWPWPLPVLLKDIEYNGPATRVRVWNPKLYASDRNHKMPIITPAYPSTCCTHNVGRSSMVVIQQELKKGASVTEEIMLGRRPWKDLFTKHTFFTSGFKYYLTVISSSRTKQAQNVWRGFIESRVRLLVNKLEMHPSIALARPFNQGYDRHDGARYTTNHYIGLQLVEGAKSLDLSREVNDWKAMCKSNELYREGLMFLSIQHLKNTALPDDVFEPGEVKPR

SEQ ID NO: 131 - protein, 490 aa, synthetic construct
MAERQWGVTPPISTALPTENEKRLHDALHKELRAQGTFESPAERAKREKVLAELEKITNVFVKRAAQEKQPKNSILIRDAIGRVFTYGSYRLGVYGPGSDIDTLVVAPKYVTVEQYFRIFPQVLVEMAPPGAIKDLTPVPEAFVPIIKFEFDGISIDLIFCSIQTLMQLPADPNWSLADNNLLRGLSENEVRSLNGTRVTDEILNLVPEPATFKLALRAIKLWAQRKAIYANIMGFPGGVAWAMLVARVCQLYPKATSAVIVNKFFHIMVKWPWPLPVLLKDIEYGCPVTRVAVWNPKLYASDRNHKMPIITPAYPSMCATHNVGNSTMAVIQQELEKGAQITDEIMVGRRPWKDLFTKHTFFTNGFKYYLTVISSSRTKQAQNAWSGFIESRVRLLVNKLEMHPDIVLARPFNKGYDRHDGARYTTNHYIGLQLVEGAKQLNLSREVNDWKAMCMSNEIYKAETMFLAIQHLKNTALPDDVFEPGEKKP

SEQ ID NO: 132 - protein, 490 aa, synthetic construct
MAERQWGVTPPISTALPTENEKRLHDALHKELRAQGTFESPAERAKREKVLAELEKITNVFVKRAAQEKQPKNSILIRDAIGRVFTHGSYRLGVYGPGSDIDTLVVAPKYVTVEQYFRIFPQVLVEMAPPGAIKDLTPVPEAFVPIIKFEFDGISIDLIFCSIQTLMQLPADPNWSLADNNLLRGLSENEVRSLNGTRVTDEILNLVPEPATFKLALRAIKLWAQRKAIYANIMGFPGGVAWAMLVARVCQLYPKATSAVIVNKFFHIMVKWPWPLPVLLKDIEYGCPVTRVAVWNPKLYASDRNHKMPIITPAYPSMCATHNVGNSTMAVIQQELEKGAQITDEIMVGRRPWKDLFTKHTFFTNGFKYYLTVISSSRTKQAQNAWSGFIESRVRLLVNKLEMHPDIVLARPFNKGYDRHDGARYTTNHYIGLQLVEGAKQLNLSREVNDWKAMCMSNEIYKAETMFLAIQHLKNTALPDDVFEPGEKKP

SEQ ID NO: 133 - protein, 490 aa, synthetic construct
MAERQWGVTPPISTALPTENEKRLHDALHKELRAQGTFESPAERAKREKVLAELEKITNVFVKRAAQEKQPKNSILIRDAIGRVFTYGSYRLGVYGPGSDIDTLVVAPKYVTVEQYFRIFPQVLVEMAPPGAIKDLTPVPEAYVPIIKFEFDGISIDLIFCSIQTLMQLPADPNWSLADNNLLRGLSENEVRSLNGTRVTDEILNLVPEPATFKLALRAIKLWAQRKAIYANIMGFPGGVAWAMLVARVCQLYPKATSAVIVNKFFHIMVKWPWPLPVLLKDIEYGCPVTRVAVWNPKLYASDRNHKMPIITPAYPSMCATHNVGNSTMAVIQQELEKGAQITDEIMVGRRPWKDLFTKHTFFTNGFKYYLTVISSSRTKQAQNAWSGFIESRVRLLVNKLEMHPDIVLARPFNKGYDRHDGARYTTNHYIGLQLVEGAKQLNLSREVNDWKAMCMSNEIYKAETMFLAIQHLKNTALPDDVFEPGEKKP

SEQ ID NO: 134 - protein, 490 aa, synthetic construct
MAERQWGVTPPISTALPTENEKRLHDALHKELRAQGTFESPAERAKREKVLAELEKITNVFVKRAAQEKQPKNSILIRDAIGRVFTYGSYRLGVYGPGSDIDTLVVAPKYVTVEQYFRIFPQVLVEMAPPGAIKDLTPVPEAFVPIINFEFDGISIDLIFCSIQTLMQLPADPNWSLADNNLLRGLSENEVRSLNGTRVTDEILNLVPEPATFKLALRAIKLWAQRKAIYANIMGFPGGVAWAMLVARVCQLYPKATSAVIVNKFFHIMVKWPWPLPVLLKDIEYGCPVTRVAVWNPKLYASDRNHKMPIITPAYPSMCATHNVGNSTMAVIQQELEKGAQITDEIMVGRRPWKDLFTKHTFFTNGFKYYLTVISSSRTKQAQNAWSGFIESRVRLLVNKLEMHPDIVLARPFNKGYDRHDGARYTTNHYIGLQLVEGAKQLNLSREVNDWKAMCMSNEIYKAETMFLAIQHLKNTALPDDVFEPGEKKP

SEQ ID NO: 135 - protein, 490 aa, synthetic construct
MAERQWGVTPPISTALPTENEKRLHDALHKELRAQGTFESPAERAKREKVLAELEKITNVFVKRAAQEKQPKNSILIRDAIGRVFTYGSYRLGVYGPGSDIDTLVVAPKYVTVEQYFRIFPQVLVEMAPPGAIKDLTPVPEAFVPIIKFEFDGISIDLIFCSIQTLMQLPADPNWSLADNNLLRGLSENEVRSLNGTRNTDEILNLVPEPATFKLALRAIKLWAQRKAIYANIMGFPGGVAWAMLVARVCQLYPKATSAVIVNKFFHIMVKWPWPLPVLLKDIEYGCPVTRVAVWNPKLYASDRNHKMPIITPAYPSMCATHNVGNSTMAVIQQELEKGAQITDEIMVGRRPWKDLFTKHTFFTNGFKYYLTVISSSRTKQAQNAWSGFIESRVRLLVNKLEMHPDIVLARPFNKGYDRHDGARYTTNHYIGLQLVEGAKQLNLSREVNDWKAMCMSNEIYKAETMFLAIQHLKNTALPDDVFEPGEKKP

SEQ ID NO: 136 - protein, 490 aa, synthetic construct
MAERQWGVTPPISTALPTENEKRLHDALHKELRAQGTFESPAERAKREKVLAELEKITNVFVKRAAQEKQPKNSILIRDAIGRVFTYGSYRLGVYGPGSDIDTLVVAPKYVTVEQYFRIFPQVLVEMAPPGAIKDLTPVPEAFVPIIKFEFDGISIDLIFCSIQTLMQLPADPNWSLADNNLLRGLSENEVRSLNGTRVTDEILRLVPEPATFKLALRAIKLWAQRKAIYANIMGFPGGVAWAMLVARVCQLYPKATSAVIVNKFFHIMVKWPWPLPVLLKDIEYGCPVTRVAVWNPKLYASDRNHKMPIITPAYPSMCATHNVGNSTMAVIQQELEKGAQITDEIMVGRRPWKDLFTKHTFFTNGFKYYLTVISSSRTKQAQNAWSGFIESRVRLLVNKLEMHPDIVLARPFNKGYDRHDGARYTTNHYIGLQLVEGAKQLNLSREVNDWKAMCMSNEIYKAETMFLAIQHLKNTALPDDVFEPGEKKP

SEQ ID NO: 137 - protein, 490 aa, synthetic construct
MAERQWGVTPPISTALPTENEKRLHDALHKELRAQGTFESPAERAKREKVLAELEKITNVFVKRAAQEKQPKNSILIRDAIGRVFTYGSYRLGVYGPGSDIDTLVVAPKYVTVEQYFRIFPQVLVEMAPPGAIKDLTPVPEAFVPIIKFEFDGISIDLIFCSIQTLMQLPADPNWSLADNNLLRGLSENEVRSLNGTRVTDEILNLVPEPATFKLALRAIKLWAQRKAIYANIMGFPGGAAWAMLVARVCQLYPKATSAVIVNKFFHIMVKWPWPLPVLLKDIEYGCPVTRVAVWNPKLYASDRNHKMPIITPAYPSMCATHNVGNSTMAVIQQELEKGAQITDEIMVGRRPWKDLFTKHTFFTNGFKYYLTVISSSRTKQAQNAWSGFIESRVRLLVNKLEMHPDIVLARPFNKGYDRHDGARYTTNHYIGLQLVEGAKQLNLSREVNDWKAMCMSNEIYKAETMFLAIQHLKNTALPDDVFEPGEKKP

SEQ ID NO: 138 - protein, 490 aa, synthetic construct
MAERQWGVTPPISTALPTENEKRLHDALHKELRAQGTFESPAERAKREKVLAELEKITNVFVKRAAQEKQPKNSILIRDAIGRVFTYGSYRLGVYGPGSDIDTLVVAPKYVTVEQYFRIFPQVLVEMAPPGAIKDLTPVPEAFVPIIKFEFDGISIDLIFCSIQTLMQLPADPNWSLADNNLLRGLSENEVRSLNGTRVTDEILNLVPEPATFKLALRAIKLWAQRKAIYANIMGFPGGVAWAVLVARVCQLYPKATSAVIVNKFFHIMVKWPWPLPVLLKDIEYGCPVTRVAVWNPKLYASDRNHKMPIITPAYPSMCATHNVGNSTMAVIQQELEKGAQITDEIMVGRRPWKDLFTKHTFFTNGFKYYLTVISSSRTKQAQNAWSGFIESRVRLLVNKLEMHPDIVLARPFNKGYDRHDGARYTTNHYIGLQLVEGAKQLNLSREVNDWKAMCMSNEIYKAETMFLAIQHLKNTALPDDVFEPGEKKP

SEQ ID NO: 139 - protein, 490 aa, synthetic construct
MAERQWGVTPPISTALPTENEKRLHDALHKELRAQGTFESPAERAKREKVLAELEKITNVFVKRAAQEKQPKNSILIRDAIGRVFTYGSYRLGVYGPGSDIDTLVVAPKYVTVEQYFRIFPQVLVEMAPPGAIKDLTPVPEAFVPIIKFEFDGISIDLIFCSIQTLMQLPADPNWSLADNNLLRGLSENEVRSLNGTRVTDEILNLVPEPATFKLALRAIKLWAQRKAIYANIMGFPGGVAWAMLVARVCQLYPKATSAVIVNKFFHIMVKWPWPLPVLLKDIEYGCPVTRVRVWNPKLYASDRNHKMPIITPAYPSMCATHNVGNSTMAVIQQELEKGAQITDEIMVGRRPWKDLFTKHTFFTNGFKYYLTVISSSRTKQAQNAWSGFIESRVRLLVNKLEMHPDIVLARPFNKGYDRHDGARYTTNHYIGLQLVEGAKQLNLSREVNDWKAMCMSNEIYKAETMFLAIQHLKNTALPDDVFEPGEKKP

SEQ ID NO: 140 - protein, 490 aa, synthetic construct
MAERQWGVTPPISTALPTENEKRLHDALHKELRAQGTFESPAERAKREKVLAELEKITNVFVKRAAQEKQPKNSILIRDAIGRVFTYGSYRLGVYGPGSDIDTLVVAPKYVTVEQYFRIFPQVLVEMAPPGAIKDLTPVPEAFVPIIKFEFDGISIDLIFCSIQTLMQLPADPNWSLADNNLLRGLSENEVRSLNGTRVTDEILNLVPEPATFKLALRAIKLWAQRKAIYANIMGFPGGVAWAMLVARVCQLYPKATSAVIVNKFFHIMVKWPWPLPVLLKDIEYGCPVTRVAVWNPKLYASDRNHKMPIITPAYPSTCATHNVGNSTMAVIQQELEKGAQITDEIMVGRRPWKDLFTKHTFFTNGFKYYLTVISSSRTKQAQNAWSGFIESRVRLLVNKLEMHPDIVLARPFNKGYDRHDGARYTTNHYIGLQLVEGAKQLNLSREVNDWKAMCMSNEIYKAETMFLAIQHLKNTALPDDVFEPGEKKP

SEQ ID NO: 141 - protein, 490 aa, synthetic construct
MAERQWGVTPPISTALPTENEKRLHDALHKELRAQGTFESPAERAKREKVLAELEKITNVFVKRAAQEKQPKNSILIRDAIGRVFTYGSYRLGVYGPGSDIDTLVVAPKYVTVEQYFRIFPQVLVEMAPPGAIKDLTPVPEAFVPIIKFEFDGISIDLIFCSIQTLMQLPADPNWSLADNNLLRGLSENEVRSLNGTRVTDEILNLVPEPATFKLALRAIKLWAQRKAIYANIMGFPGGVAWAMLVARVCQLYPKATSAVIVNKFFHIMVKWPWPLPVLLKDIEYGCPVTRVAVWNPKLYASDRNHKMPIITPAYPSMCCTHNVGNSTMAVIQQELEKGAQITDEIMVGRRPWKDLFTKHTFFTNGFKYYLTVISSSRTKQAQNAWSGFIESRVRLLVNKLEMHPDIVLARPFNKGYDRHDGARYTTNHYIGLQLVEGAKQLNLSREVNDWKAMCMSNEIYKAETMFLAIQHLKNTALPDDVFEPGEKKP

SEQ ID NO: 142 - protein, 490 aa, synthetic construct
MAERQWGVTPPISTALPTENEKRLHDALHKELRAQGTFESPAERAKREKVLAELEKITNVFVKRAAQEKQPKNSILIRDAIGRVFTYGSYRLGVYGPGSDIDTLVVAPKYVTVEQYFRIFPQVLVEMAPPGAIKDLTPVPEAFVPIIKFEFDGISIDLIFCSIQTLMQLPADPNWSLADNNLLRGLSENEVRSLNGTRNTDEILNLVPEPATFKLALRAIKLWAQRKAIYANIMGFPGGAAWAVLVARVCQLYPKATSAVIVNKFFHIMVKWPWPLPVLLKDIEYGCPVTRVAVWNPKLYASDRNHKMPIITPAYPSTCATHNVGNSTMAVIQQELEKGAQITDEIMVGRRPWKDLFTKHTFFTNGFKYYLTVISSSRTKQAQNAWSGFIESRVRLLVNKLEMHPDIVLARPFNKGYDRHDGARYTTNHYIGLQLVEGAKQLNLSREVNDWKAMCMSNEIYKAETMFLAIQHLKNTALPDDVFEPGEKKP

SEQ ID NO: 143 - protein, 490 aa, synthetic construct
MAERQWGVTPPISTALPTENEKRLHDALHKELRAQGTFESPAERAKREKVLAELEKITNVFVKRAAQEKQPKNSILIRDAIGRVFTYGSYRLGVYGPGSDIDTLVVAPKYVTVEQYFRIFPQVLVEMAPPGAIKDLTPVPEAYVPIIKFEFDGISIDLIFCSIQTLMQLPADPNWSLADNNLLRGLSENEVRSLNGTRNTDEILRLVPEPATFKLALRAIKLWAQRKAIYANIMGFPGGAAWAVLVARVCQLYPKATSAVIVNKFFHIMVKWPWPLPVLLKDIEYGCPVTRVRVWNPKLYASDRNHKMPIITPAYPSTCCTHNVGNSTMAVIQQELEKGAQITDEIMVGRRPWKDLFTKHTFFTNGFKYYLTVISSSRTKQAQNAWSGFIESRVRLLVNKLEMHPDIVLARPFNKGYDRHDGARYTTNHYIGLQLVEGAKQLNLSREVNDWKAMCMSNEIYKAETMFLAIQHLKNTALPDDVFEPGEKKP

SEQ ID NO: 144 - protein, 490 aa, synthetic construct
MAERQWGVTPPISTALPTENEKRLHDALHKELRAQGTFESPAERAKREKVLAELEKITNVFVKRAAQEKQPKNSILIRDAIGRVFTHGSYRLGVYGPGSDIDTLVVAPKYVTVEQYFRIFPQVLVEMAPPGAIKDLTPVPEAYVPIIKFEFDGISIDLIFCSIQTLMQLPADPNWSLADNNLLRGLSENEVRSLNGTRNTDEILRLVPEPATFKLALRAIKLWAQRKAIYANIMGFPGGAAWAVLVARVCQLYPKATSAVIVNKFFHIMVKWPWPLPVLLKDIEYGCPVTRVRVWNPKLYASDRNHKMPIITPAYPSTCCTHNVGNSTMAVIQQELEKGAQITDEIMVGRRPWKDLFTKHTFFTNGFKYYLTVISSSRTKQAQNAWSGFIESRVRLLVNKLEMHPDIVLARPFNKGYDRHDGARYTTNHYIGLQLVEGAKQLNLSREVNDWKAMCMSNEIYKAETMFLAIQHLKNTALPDDVFEPGEKKP

SEQ ID NO: 145 - protein, 490 aa, synthetic construct
MAERQWGVTPPISTALPTENEKRLHDALHKELRAQGTFESPAERAKREKVLAELEKITNVFVKRAAQEKQPKNSILIRDAIGRVFTHGSYRLGVYGPGSDIDTLVVAPKYVTVEQYFRIFPQVLVEMAPPGAIKDLTPVPEAYVPIINFEFDGISIDLIFCSIQTLMQLPADPNWSLADNNLLRGLSENEVRSLNGTRNTDEILRLVPEPATFKLALRAIKLWAQRKAIYANIMGFPGGAAWAVLVARVCQLYPKATSAVIVNKFFHIMVKWPWPLPVLLKDIEYGCPVTRVRVWNPKLYASDRNHKMPIITPAYPSTCCTHNVGNSTMAVIQQELEKGAQITDEIMVGRRPWKDLFTKHTFFTNGFKYYLTVISSSRTKQAQNAWSGFIESRVRLLVNKLEMHPDIVLARPFNKGYDRHDGARYTTNHYIGLQLVEGAKQLNLSREVNDWKAMCMSNEIYKAETMFLAIQHLKNTALPDDVFEPGEKKP

SEQ ID NO: 146 - protein, 658 aa, synthetic construct
MTERTYGVTPPISTALPTEQEKALNKALHDELRAQGTFESRAETEKRKEVLAQLEKITNAFVQRAAREKHGKNAILIRDAIGRVFTYGSYRLGVYGPGSDMDTLVVAPKYVTVEQYFRIFPEVLVEMAPPAAITDLTPVPEAFVPIIKFEFSGISIDLIFCSIQTLIQLPADKSWSLADNNLLRGLSENAVRSLNGTRVTDEILHLVPEPATFKLALRAIKLWAQRKAIYANIMGYPGGVAWAMLVARVCQLYPKATSAVIVNKFFNIMLKWPWPLPVLLKDIEYNGPVTRVPVWNPKLYASDRNHKMPIITPAYPSMCATHNVGRSSMVVIQQELKKGAEVTEEIMLGRRPWKDLFTKHTFFTSGFKYYLTVISSSRTKKAQNVWSGFIESRVRLLVNKIEMHPSIALARPFNKGYDRMHRCKNDAQVEEVVSAGSLAYVYTPPAFGDEKVKSETKSEVKQEVKQEVRQDDVIQDGVPVKQEKAEVRAEDGVRIKRELSEEVQLPPPTNVKPEPHDDEAKVKLEDIPEKEAQPEDMEIYTTNHYIGLQLVEGAKSLDLSREVNDWKAMCMSNELYEEGLMFLSIQHLKNTALPDDVFEPGEVKPRPGKKVLKRGASEEPSKQQPPAKRQAHVQPRAPAAQQPSSTATAAAGHHHHHH

SEQ ID NO: 147 - protein, 496 aa, synthetic construct
MTERTYGVTPPISTALPTEQEKALNKALHDELRAQGTFESRAETEKRKEVLAQLEKITNAFVQRAAREKHGKNAILIRDAIGRVFTYGSYRLGVYGPGSDMDTLVVAPKYVTVEQYFRIFPEVLVEMAPPAAITDLTPVPEAFVPIIKFEFSGISIDLIFCSIQTLIQLPADKSWSLADNNLLRGLSENAVRSLNGTRVTDEILHLVPEPATFKLALRAIKLWAQRKAIYANIMGYPGGVAWAMLVARVCQLYPKATSAVIVNKFFNIMLKWPWPLPVLLKDIEYNGPVTRVPVWNPKLYASDRNHKMPIITPAYPSMCATHNVGRSSMVVIQQELKKGAEVTEEIMLGRRPWKDLFTKHTFFTSGFKYYLTVISSSRTKKAQNVWSGFIESRVRLLVNKIEMHPSIALARPFNKGYDRHDGARYTTNHYIGLQLVEGAKSLDLSREVNDWKAMCMSNELYEEGLMFLSIQHLKNTALPDDVFEPGEVKPHHHHHH

SEQ ID NO: 148 - protein, 497 aa, synthetic construct
MAERTYGVTPPISTALPTEQEKALNKALHDELRAQGTFESPAETEKRKEVLAQLEKITNAFVQRAAREKHPKNAILIRDAIGRVFTHGSYRLGVYGPGSDMDTLVVAPKYVTVEQYFRIFPEVLVEMAPPGAITDLTPVPEAYVPIIKFEFSGISIDLIFCSIQTLIQLPADKSWSLADNNLLRGLSENAVRSLNGTRNTDEILRLVPEPATFKLALRAIKLWAQRKAIYANIMGYPGGAAWAVLVARVCQLYPKATSAVIVNKFFNIMLKWPWPLPVLLKDIEYNGPATRVRVWNPKLYASDRNHKMPIITPAYPSTCCTHNVGRSSMVVIQQELKKGASVTEEIMLGRRPWKDLFTKHTFFTSGFKYYLTVISSSRTKKAQNVWSGFIESRVRLLVNKIEMHPSIALARPFNKGYDRHDGARYTTNHYIGLQLVEGAKSLDLSREVNDWKAMCMSNELYEEGLMFLSIQHLKNTALPDDVFEPGEVKPRHHHHHH

SEQ ID NO: 149 - protein, 603 aa, Phaeoacremonium minimum
  source 1..603: note = UCRPA7
MTSEKQYGTTPPISVTLPTEGEKQSTESLFRELKAQGTFEHATETQKREKVLASIQAIADVFVKKVAKEREPENALLARDARGHVFTYGSYRLGVYGPGSDIDTLVVAPRYVTREDFFKHFPGLLKEMTPPGSIEDLAAVTDAFVPIIKFEYSGISIDLIFSRIATLKQLPPRDKFNLLDNNLLRGLDEAELRSLNGTRVTDEILELVPEKTSFRLALRAIKLWAQRRAIYANIMGYPGGVAWAMLVARISQLYPKATGAVIVNKFFNIMRRWPWPQPVLLKHIEDGPLQVRVWNPKIYKGDTFHLMPIITPAYPSMCATFNVTKSAMAVIQKELDTFAEVTDQIMLGKRPWKDLFVKHTFFTSGFKYYIAVISASKTKEAHKLWSGFVESKIRLLVQGIERHPSIALARPFNKGYERTHRCKTGAEVNKVQDGDLSFVLKEAEIVEQKATTEVAGVEVAGTEVKAADGNGEEAAKPNGTETETKEETIDVYTTTHYIGLELYEGAKSLDLSYQVNEFKDLCHNWDKYKDELNEHCSLSIQHVRNINLPDDVFEAGEVKPTKPIKKKVPATNGAAKKRGASERQRNANKHQLLRLAEHGRELR

SEQ ID NO: 150 - protein, 609 aa, synthetic construct
MTSEKQYGTTPPISVTLPTEGEKQSTESLFRELKAQGTFEHATETQKREKVLASIQAIADVFVKKVAKEREPENALLARDARGHVFTYGSYRLGVYGPGSDIDTLVVAPRYVTREDFFKHFPGLLKEMTPPGSIEDLAAVTDAFVPIIKFEYSGISIDLIFSRIATLKQLPPRDKFNLLDNNLLRGLDEAELRSLNGTRVTDEILELVPEKTSFRLALRAIKLWAQRRAIYANIMGYPGGVAWAMLVARISQLYPKATGAVIVNKFFNIMRRWPWPQPVLLKHIEDGPLQVRVWNPKIYKGDTFHLMPIITPAYPSMCATFNVTKSAMAVIQKELDTFAEVTDQIMLGKRPWKDLFVKHTFFTSGFKYYIAVISASKTKEAHKLWSGFVESKIRLLVQGIERHPSIALARPFNKGYERTHRCKTGAEVNKVQDGDLSFVLKEAEIVEQKATTEVAGVEVAGTEVKAADGNGEEAAKPNGTETETKEETIDVYTTTHYIGLELYEGAKSLDLSYQVNEFKDLCHNWDKYKDELNEHCSLSIQHVRNINLPDDVFEAGEVKPTKPIKKKVPATNGAAKKRGASERQRNANKHQLLRLAEHGRELRHHHHHH

SEQ ID NO: 151 - protein, 499 aa, synthetic construct
MTSEKQYGTTPPISVTLPTEGEKQSTESLFRELKAQGTFEHATETQKREKVLASIQAIADVFVKKVAKEREPENALLARDARGHVFTYGSYRLGVYGPGSDIDTLVVAPRYVTREDFFKHFPGLLKEMTPPGSIEDLAAVTDAFVPIIKFEYSGISIDLIFSRIATLKQLPPRDKFNLLDNNLLRGLDEAELRSLNGTRVTDEILELVPEKTSFRLALRAIKLWAQRRAIYANIMGYPGGVAWAMLVARISQLYPKATGAVIVNKFFNIMRRWPWPQPVLLKHIEDGPLQVRVWNPKIYKGDTFHLMPIITPAYPSMCATFNVTKSAMAVIQKELDTFAEVTDQIMLGKRPWKDLFVKHTFFTSGFKYYIAVISASKTKEAHKLWSGFVESKIRLLVQGIERHPSIALARPFNKGYERHDGARYTTTHYIGLELYEGAKSLDLSYQVNEFKDLCHNWDKYKDELNEHCSLSIQHVRNINLPDDVFEAGEVKPTHHHHHH

SEQ ID NO: 152 - protein, 498 aa, synthetic construct
MAEKQYGTTPPISVTLPTEGEKQSTESLFRELKAQGTFEHPTETQKREKVLASIQAIADVFVKKVAKEREPENALLARDARGHVFTHGSYRLGVYGPGSDIDTLVVAPRYVTREDFFKHFPGLLKEMTPPGSIEDLAAVTDAYVPIIKFEYSGISIDLIFSRIATLKQLPPRDKFNLLDNNLLRGLDEAELRSLNGTRNTDEILRLVPEKTSFRLALRAIKLWAQRRAIYANIMGYPGGAAWAVLVARISQLYPKATGAVIVNKFFNIMRRWPWPQPVLLKHIEDGPLQVRVWNPKIYKGDTFHLMPIITPAYPSTCCTFNVTKSAMAVIQKELDTFASVTDQIMLGKRPWKDLFVKHTFFTSGFKYYIAVISASKTKEAHKLWSGFVESKIRLLVQGIERHPSIALARPFNKGYERHDGARYTTTHYIGLELYEGAKSLDLSYQVNEFKDLCHNWDKYKDELNEHCSLSIQHVRNINLPDDVFEAGEVKPTHHHHHH

SEQ ID NO: 153 - protein, 588 aa, Ophiostoma piceae
  source 1..588: note = UAMH 11346
MNFEKQYGVTPPLSTEMPTEEQRRATDALLEELRNQGTFESAAETQKRYKVLESLGAVADAFVKRVAREKAPDNPTLSKDAIAKIFTYGSFRLGVYMPGSDIDTLVLAPKYVTREDYFKYFPEMLQAMSPPGSITDLTAVEDAFVPIIKFEYSDISIDLIFSRVLLTQLPSDPNWGLNDSNLLRGLDEAELRSVNGTRVTDEILALVPEQRTFRDALRAIKLWAQRRAIYANIMGFPGGVAWAMMVARVCQLYPKATGAVIVSKFFGIILRWPWPNPVLLKPIEDGPLPVRVWNPKIYRGDQFHLMPIITPAYPSMCATYNISQSSLSIIKRELERASETAQQVIGGRQPWKDLFTKHTFFTCDFKYYISVISASTNKDDQNTWSGFVESKVRVLVQGLERHQSIFLARAFNKGYERTHLCKNDEEVTLVQDGSMNFLKKDDAESSGKEKEGDNIKVYTLTHYIGIELVSGAKSLDLSHEVKEFKDLCNAWEKYKETLEKTCSLSIQHVRNIGLPDDVFEPGEVKPAKPVRAARPGRRNPPTGNNGTKRRIVSEDAAPPSKRQQSQAPARNENGQQQPPSTVVAAAGG

SEQ ID NO: 154 - protein, 594 aa, synthetic construct
MNFEKQYGVTPPLSTEMPTEEQRRATDALLEELRNQGTFESAAETQKRYKVLESLGAVADAFVKRVAREKAPDNPTLSKDAIAKIFTYGSFRLGVYMPGSDIDTLVLAPKYVTREDYFKYFPEMLQAMSPPGSITDLTAVEDAFVPIIKFEYSDISIDLIFSRVLLTQLPSDPNWGLNDSNLLRGLDEAELRSVNGTRVTDEILALVPEQRTFRDALRAIKLWAQRRAIYANIMGFPGGVAWAMMVARVCQLYPKATGAVIVSKFFGIILRWPWPNPVLLKPIEDGPLPVRVWNPKIYRGDQFHLMPIITPAYPSMCATYNISQSSLSIIKRELERASETAQQVIGGRQPWKDLFTKHTFFTCDFKYYISVISASTNKDDQNTWSGFVESKVRVLVQGLERHQSIFLARAFNKGYERTHLCKNDEEVTLVQDGSMNFLKKDDAESSGKEKEGDNIKVYTLTHYIGIELVSGAKSLDLSHEVKEFKDLCNAWEKYKETLEKTCSLSIQHVRNIGLPDDVFEPGEVKPAKPVRAARPGRRNPPTGNNGTKRRIVSEDAAPPSKRQQSQAPARNENGQQQPPSTVVAAAGGHHHHHH

SEQ ID NO: 155 - protein, 498 aa, synthetic construct
MNFEKQYGVTPPLSTEMPTEEQRRATDALLEELRNQGTFESAAETQKRYKVLESLGAVADAFVKRVAREKAPDNPTLSKDAIAKIFTYGSFRLGVYMPGSDIDTLVLAPKYVTREDYFKYFPEMLQAMSPPGSITDLTAVEDAFVPIIKFEYSDISIDLIFSRVLLTQLPSDPNWGLNDSNLLRGLDEAELRSVNGTRVTDEILALVPEQRTFRDALRAIKLWAQRRAIYANIMGFPGGVAWAMMVARVCQLYPKATGAVIVSKFFGIILRWPWPNPVLLKPIEDGPLPVRVWNPKIYRGDQFHLMPIITPAYPSMCATYNISQSSLSIIKRELERASETAQQVIGGRQPWKDLFTKHTFFTCDFKYYISVISASTNKDDQNTWSGFVESKVRVLVQGLERHQSIFLARAFNKGYERHDGARYTLTHYIGIELVSGAKSLDLSHEVKEFKDLCNAWEKYKETLEKTCSLSIQHVRNIGLPDDVFEPGEVKPAHHHHHH

SEQ ID NO: 156 - protein, 497 aa, synthetic construct
MAEKQYGVTPPLSTEMPTEEQRRATDALLEELRNQGTFESPAETQKRYKVLESLGAVADAFVKRVAREKAPDNPTLSKDAIAKIFTHGSFRLGVYMPGSDIDTLVLAPKYVTREDYFKYFPEMLQAMSPPGSITDLTAVEDAYVPIIKFEYSDISIDLIFSRVLLTQLPSDPNWGLNDSNLLRGLDEAELRSVNGTRNTDEILRLVPEQRTFRDALRAIKLWAQRRAIYANIMGFPGGAAWAVMVARVCQLYPKATGAVIVSKFFGIILRWPWPNPVLLKPIEDGPLPVRVWNPKIYRGDQFHLMPIITPAYPSTCCTYNISQSSLSIIKRELERASSTAQQVIGGRQPWKDLFTKHTFFTCDFKYYISVISASTNKDDQNTWSGFVESKVRVLVQGLERHQSIFLARAFNKGYERHDGARYTLTHYIGIELVSGAKSLDLSHEVKEFKDLCNAWEKYKETLEKTCSLSIQHVRNIGLPDDVFEPGEVKPAHHHHHH

SEQ ID NO: 157 - protein, 5 aa, synthetic construct
  REGION 1..5: note = HDGAR loop
HDGAR

SEQ ID NO: 158 - protein, 446 aa, synthetic construct
LHDELRAQGTFESPAETEKRKEVLAQLEKITNAFVQRAAREKHPKNAILIRDAIGRVFTHGSYRLGVYGPGSDMDTLVVAPKYVTVEQYFRIFPEVLVEMAPPGAITDLTPVPEAYVPIIKFEFSGISIDLIFCSIQTLIQLPADKSWSLADNNLLRGLSENAVRSLNGTRNTDEILRLVPEPATFKLALRAIKLWAQRKAIYANIMGYPGGAAWAVLVARVCQLYPKATSAVIVNKFFNIMLKWPWPLPVLLKDIEYNGPATRVRVWNPKLYASDRNHKMPIITPAYPSTCCTHNVGRSSMVVIQQELKKGASVTEEIMLGRRPWKDLFTKHTFFTSGFKYYLTVISSSRTKKAQNVWSGFIESRVRLLVNKIEMHPSIALARPFNKGYDRHDGARYTTNHYIGLQLVEGAKSLDLSREVNDWKAMCMSNELYEEGLMFLSIQHL

SEQ ID NO: 159 - protein, 491 aa, synthetic construct; identical to SEQ ID NO: 127